(19) United States
(12) Patent Application Publication
(10) Pub. No.: US 2019/0282731 A1
(43) Pub. Date: Sep. 19, 2019

(54) Enzymatically Crosslinked Compositions

(71) Applicant: Trustees of Tufts College, Medford, MA (US)

(72) Inventors: Nicole R. Raia, Maynard, MA (US); David L. Kaplan, Concord, MA (US); Benjamin P. Partlow, Winchester, MA (US)

(21) Appl. No.: 16/463,762

(22) PCT Filed: Nov. 22, 2017
(86) PCT No.: PCT/US2017/063041

§ 371 Date: May 23, 2019

Related U.S. Application Data
(60) Provisional application No. 62/425,883, filed on Nov. 23, 2016.

(51) Int. Cl. A61L 27/26; A61L 27/36; A61L 27/52; A61L 27/54; A61K 47/42; A61K 47/46; A61K 47/36

(52) U.S. Cl. CPC A61L 27/26; A61L 27/3604; A61L 27/52; C08L 89/00; A61K 47/42; A61K 47/46; A61K 47/36; A61L 27/54

(57) ABSTRACT

In some embodiments, the present disclosure provides compositions including silk fibroin and a phenol-containing polymer, wherein at least one tyrosine group of the silk fibroin is covalently crosslinked to at least one phenol group of the phenol-containing polymer. In some embodiments, the present invention also provides methods including the steps of providing silk fibroin, providing a phenol-containing polymer, associating the silk fibroin with the phenol-containing polymer to form a mixed solution, and crosslinking at least one tyrosine group in the silk fibroin and at least one phenol group of the phenol-containing polymer via at least one enzymatic reaction, wherein the crosslinking comprises covalent bonding between at least one tyrosine group of the silk fibroin and at least one phenol group of the phenol-containing polymer to form a crosslinked composition.

### CROSS-REFERENCE TO RELATED APPLICATIONS

This Application claims the benefit under 35 U.S.C § 119(e) of U.S. Provisional Application No. 62/425883 filed Nov. 23, 2016, the content of which is incorporated herein by reference in its entirety.

### GOVERNMENT SUPPORT

This invention was made with government support under grant No. R01 EB02126, P41 EB002520 awarded by the National Institutes of Health, and grant No. FA9550-14-1-0015 awarded by the Air Force Office of Scientific Research. The government has certain rights in the invention.

### BACKGROUND

There is a continuing need for improved compositions for use in, inter alia, biomedical applications, including regenerative medicine, drug delivery, and tissue engineering. In some applications, hydrogels have proven to be desirable compositions due in part to their typical hydrophilic nature, permeability to oxygen and nutrients, and mechanical properties. However, several limitations of previously known hydrogels, including tendency to become brittle over time, limit their effective use.

### SUMMARY

The present invention offers, among other things, biocompatible compositions with previously unattainable advantages including, without limitation, the mechanical integrity of silk fibroin combined with the desirable characteristics (e.g., hydrophilicity, bioactivity, etc) of certain phenol-containing polymers. In part, aspects of the present invention overcome one or more limitations in other prior compositions (including certain silk fibroin-containing compositions) wherein the resultant compositions: were too brittle for a variety of applications, were lacking in bioactivity (e.g., by virtue of not including a phenol-containing bioactive polymer); and/or were vulnerable to increased crystallization over time (resulting in decreasing flexibility and/or other undesired mechanical changes over time). In accordance with a variety of embodiments, it is contemplated that provided methods and compositions may be useful in, inter alia, cell encapsulation, tissue engineering, and/or delivery of one or more active agents (e.g., therapeutic agents).

In some embodiments, the present disclosure provides compositions including silk fibroin and a phenol-containing polymer, wherein at least one tyrosine group of the silk fibroin is covalently crosslinked to at least one phenol group of the phenol-containing polymer.

The present invention also provides, among other things, novel compositions and methods for making and using provided compositions. In some embodiments, provided composite hydrogels comprise enzymatically crosslinked silk fibroin and tyramine (or tyrosine)-substituted polymer(s) (e.g., hyaluronic acid). In some embodiments, these hybrid, biocompatible, hydrogel systems offer previously unattainable advantages, for example, the mechanical integrity of silk combined with the hydrophilicity and bioactivity of hyaluronic acid. As described herein, certain exemplary characterizations were focused on how the polymer concentrations affected the physical properties of the gels over time. Certain exemplary results described herein show that increasing concentrations of hyaluronic acid delays and decreases the amount of stiffening and crystallization as determined through dynamic mechanical analysis (DMass.) and Fourier transform infrared spectroscopy (FTIR). Other provided exemplary characterization techniques include gelation and swelling kinetics, rheological properties, liquid chromatography-mass spectroscopy (LC-MS), and opacity.

In some embodiments, the present invention also provides methods including the steps of providing silk fibroin, providing a phenol-containing polymer, associating the silk fibroin with the phenol-containing polymer to form a mixed solution, and crosslinking at least one tyrosine group in the silk fibroin and at least one phenol group of the phenol-containing polymer via at least one enzymatic reaction, wherein the crosslinking comprises covalent bonding between at least one tyrosine group of the silk fibroin and at least one phenol group of the phenol-containing polymer to form a crosslinked composition. In some embodiments, the silk fibroin and phenol-containing polymer are each provided in a separate solution prior to the associating step. Without wishing to be held to a particular theory, in some embodiments, attempting to solubilize silk fibroin with at least one phenol-containing polymer may result in aggregation and/or precipitation of the silk and/or inability to fully solubilize the phenol-containing polymer.

In accordance with a variety of embodiments, any application-appropriate silk fibroin may be used. In some embodiments, silk fibroin is selected from the group consisting of silkworm silk fibroin, spider silk fibroin, and recombinant silk fibroin.

In some embodiments, any application-appropriate phenol-containing polymer may be used. The specific phenol-containing polymer(s) used in a particular embodiment may depend on one or more of: the specific application of a provided composition, the physical or mechanical properties desired in the resultant composition, or the desired time to gelation for a particular embodiment (e.g., if encapsulation of one or more active agents is desired, it may be advantageous to have rapid gelation occur, such within one minute or less from the initiation of a crosslinking step). In some embodiments, a phenol-containing polymer is or comprises a peptide or protein. In some embodiments, a phenol-containing polymer is or comprises a tyramine-containing and/or tyrosine-containing peptide or protein. In some embodiments, a tyramine-containing and/or tyrosine-containing polymer is or comprises hyaluronic acid and/or polyethylene glycol. In some embodiments, a tyramine-containing and/or tyrosine-containing polymer comprises a modified form (e.g., wherein one or more phenol or tyramine group(s) added) of one or more of: dopamine, L-DOPA, serotonin, adrenaline, noradrenaline, salicylic acid, alginate, dextran, collagen, gelatin, chitosan, carboxymethylcellulose, heparin, poly(vinyl alcohol), sugars (e.g., lactose, cellulose, mannose, galactose, glucose, maltose, etc) or dimers or trimers thereof.

In contrast to many previously known crosslinked silk compositions and methods of making them, various embodiments of the present invention avoid the use of toxic reagents and/or toxic modifications to components of provided compositions, thus allowing for truly biocompatible compositions. In some embodiments, a provided composition may additionally be biodegradable.

In accordance with various embodiments, provided compositions may take any of several forms. In some embodiments, a provided composition may be or comprise a hydrogel. In some embodiments, a provided hydrogel may further include an additional structure such as a tube, particle, film, foam, etc. In some embodiments, a provided composition may be partially or totally encapsulated in an additional structure. In some embodiments, a provided composition may partially or totally encapsulate an additional structure.

In some embodiments, a provided composition may further include at least one of an active agent and a plurality of particles. In some embodiments, an active agent may be or comprise a peptide, a protein, an antibody, an enzyme, an amino acid, a nucleic acid (e.g., polynucleotides, oligonucleotides, genes, genes including control and termination regions, antisense oligonucleotides, aptamers), a nucleotide, a metabolite, a lipid, a sugar, a glycoprotein, a peptidoglycan, a microbe, a cell, and any combinations thereof. In some embodiments, an active agent may be or comprise a biologically active peptide, for example, a peptide that facilitates and/or enhances at least one of cell attachment, call growth, and cellular differentiation. In some embodiments, a peptide is or comprises a biodegradable peptide.

Depending upon the desired application and/or desired mechanical or physical properties of a particular composition, any of a variety of amounts of phenol-containing polymer may be included. In some embodiments, provided compositions may include an amount of phenol-containing polymer between 2.5 mg/mL and 200 mg/mL. In some embodiments, an amount of phenol-containing polymer may be at most 8.5 mg/mL.

In accordance with various embodiments, any of a variety of enzymes may be used to crosslink silk fibroin with one or more phenol-containing polymers. In some embodiments, the enzyme is or comprises a peroxidase. In some embodiments, a peroxidase may be or comprise a plant-based or mammal-based peroxidase. In some embodiments, an enzyme may be or comprise at least one of hydrogen peroxide, tyrosinase, laccase, hemin, a microperoxidase, cytochrome c, porphyrins, fenton, soy bean peroxidase, myeloperoxidase, lactoperoxidase, eosinophil peroxidase, thyroid peroxidase, prostaglandin H synthase, and horseradish peroxidase. In some embodiments, silk fibroin and a phenol-containing polymer are crosslinked directly to one another (e.g., there is no spacer between the two, for example, no epoxide or multiamine spacer). In some embodiments, crosslinking in provided methods and compositions is or comprises multi-phenol crosslinks. In some embodiments, multi-phenol crosslinks are selected from the group consisting of di-tyrosine crosslinks, di-tyramine crosslinks, and tyrosine-tyramine crosslinks. In some embodiments, crosslinking does not include physical crosslinking (e.g., via β-sheet formation). In some embodiments wherein the crosslinking does not include physical crosslinking, provided compositions or methods may include β-sheet formation during a later time or step subsequent to a crosslinking step. In some embodiments, crosslinking does not include chemical crosslinking (e.g., using harsh or toxic chemicals, via addition reaction(s), exposure to high energy radiation such as gamma rays or electron beams). In some embodiments, crosslinking may comprise one or more of a condensation reaction(s), carbodiimide crosslinking, and glutaraldehyde crosslinking.

In accordance with various embodiments, silk fibroin may be modified prior to use in provided methods and compositions. For example, in some embodiments, silk fibroin may be modified to include more tyrosine groups than native silk fibroin (e.g., silk fibroin from a silkworm or a spider), or to include fewer tyrosine groups than native silk fibroin. By way of specific example, in some embodiments, silk fibroin may be modified to include at least one non-native tyrosine. In some embodiments, silk fibroin may be modified to remove or alter at least one tyrosine group from native silk fibroin such that it is no longer able to crosslink with a phenol-containing polymer. In some embodiments, addition of tyrosine groups to silk fibroin may occur via carbodiimide chemistry. Any known technique for quantifying the amount of tyrosine groups on silk fibroin may be used. For example, in some embodiments, quantification of tyrosine groups may be performing using one or more of spectrophotometric analysis (e.g., via UV absorbance), liquid chromatography-mass spectrometry (LC-MS), and high performance liquid chromatography (HPLC).

As described herein, it is specifically contemplated that various embodiments may take any application-appropriate form. For example, in some embodiments, a provided composition may be formulated for administration to a subject (e.g., via injection, implantation, insertion via a cannula or catheter, etc). In some embodiments, for example where a provided composition is formulated for injection, a provided composition may have an injection force of at most 50 N (e.g., at most 40N, 30N, 20N, 10N, or less).

In accordance with various embodiments, provided compositions may exhibit any of a range of gelation times. For example, where a slower gelation time is desired for a particular application, a provided composition may be tuned to gel over a period of approximately 30 minutes to two hours (e.g., between 30 minutes and one hour, between 30 minutes and 90 minutes, or between one hour and two hours). For example, where a slower gelation time is desired for a particular application, a provided composition may be tuned to gel over a period of approximately 30 minutes or less (e.g., 25 minutes, 20 minutes, 15 minutes, 10 minutes, 5 minutes, or less). In some embodiments, a provided hydrogel exhibits a gelation time of between 10 seconds and 20 minutes after the crosslinking step.

In accordance with several embodiments, provided compositions may exhibit any of a variety of desirable properties. For example, in some embodiments, a provided composition has a compressive moduli of between 200 Pa and 1 MPa (e.g., between 200 Pa and 500 kPa). In some embodiments, a provided composition has a mass fraction of at most 1.00 after soaking in an aqueous solution for 12 hours. In some embodiments, provided compositions may have mass fractions of between 0.8-0.99 after soaking in an aqueous solution for 12 hours.

### BRIEF DESCRIPTION OF THE DRAWING

The foregoing and other objects, aspects, features, and advantages of the present disclosure will become more apparent and better understood by referring to the following description taken in conjunction with the accompanying figures in which:

FIG. 1 Gelation Kinetics. Panel (a) shows gelation time as determined via the inverted tube test showed that increasing HA concentration above that of 1% HA significantly decreased gelation time. HA content also affected crosslinking kinetics as determined through fluorescence spectroscopy (315 nm/415 nm) (see panels b-d). HA added to the hydrogels decreased the time at which crosslinking was complete (see panel b) and also decreased the lag period seen most prominently in the 0% sample during the earlier time points (see panel c). The crosslinking kinetics of HA only samples (panel d) experienced no delay and plateaued were significantly faster than their hybrid hydrogel equivalents. (a) n=4, (b-d) n=6, *p≤0.05, **p≤0.01, ***p≤0.001.

FIG. 2 Gelation Kinetics. Panel (a) shows gelation time as determined via the inverted tube test showed samples prepared with 0.5× PBS gelled much slower than that of samples prepared with water except for hydrogels with 30% HA which had similar gelation times. PBS also affected the crosslinking kinetics as determined through fluorescence spectroscopy (315 nm/415 nm) (see panels b-d). PBS increased the time at which crosslinking was complete (see panel b). A delay before rapid crosslinking is seen with samples below that of 10% HA (see panel c). The crosslinking kinetics of HA only samples (panel d) experienced no delay and finished crosslinking significantly faster than their hybrid hydrogel equivalents. (a) n=4, (b-d) n=6, *p≤0.05, **p≤0.01, ***p≤0.001.

FIG. 3 Rheological Properties. Panel (a) shows representative time sweeps, panel (b) shows strain sweeps, and panel (c) shows final storage moduli show that silk-HA composites reach a plateau slower, have a higher final storage modulus, and a higher strain at failure as compared to hydrogels that only contain HA (see panel d). All hydrogels tested showed frequency independence. n=3, *p≤0.05, **p≤0.01, ***p≤0.001.

FIG. 4 shows exemplary comparisons between covalent crosslinks as determined by calculating the peak area from liquid chromatography-mass spectrometry (LC-MS) n=1.

FIG. 5 Unconfined Compression. Panel (a) shows the compressive tangent moduli of the hydrogels were dependent on HA concentration over time, with a higher percentage of HA lead to a lower modulus at week 4. Panel (b) shows the fraction of the initial modulus (relative modulus) decreases with increasing HA content. Hydrogels containing 0% and 10% HA have relative moduli of 187.29±22.94 and 24.52±5.44, respectively. At week 4, all conditions had compressive and relative moduli that were statistically different compared to the 0% HA hydrogel (p<0.001; see panels c and d). Initial and week 4 stress-strain curves show that stress and hysteresis increase for all samples increase over time (see panel d). The curves at week 4 show that increasing HA content lead to a decreased amount of hysteresis.

FIG. 6 Fourier-transform infrared spectroscopy (FTIR) Absorbance Spectra. This figure shows the average absorbance spectrum in the amide I region is shown at weeks 0, 2, and 4. The peak at ˜1620 cm⁻¹, which is representative of beta sheet formation, becomes much larger and sharper as HA concentration decreases. n=5.

FIG. 7 Swelling Properties. The fraction of initial mass was determined for hydrogel samples over 12 hours after soaking in: ultrapure water (panel a) and 1× PBS (panel b) at 37° C. Above 0.5% HA, the hydrogels experienced an initial increase in mass followed by a decrease and final plateau value at 12 hrs that was directly related to HA concentration. Samples in 1× PBS had a decrease in mass initially and plateaued after 6 hrs. Final plateau value increased with increasing HA concentration except for that of 30% HA. n=5.

FIG. 8 Opacity. Absorbance intensity at 550 nm (panel a), fraction of initial absorbance (panel b) and images showing hydrogels (panel c) overtime showing the opacity differences over time. Initially, opacity increased with increasing HA content. The fraction of initial opacity decreased closer to 1 with higher HA content suggesting that the opacity remained more constant over time. n=6.

FIG. 9 Fluorescence Kinetics. The rate of the formation of crosslinking was determined by monitoring the fluorescence at excitation/emission 315/415 nm. Age of the 1% hydrogen peroxide has a significant effect on crosslinking kinetics where older hydrogen peroxide (4 days) showed a much slower increase in fluorescence as compared to a freshly made solution.

FIG. 10 BCA Assay. Results from an exemplary BCA assay show that there are interfering substances that produce false signals. This is seen in the protein concentration that was detected in negative controls with no protein (HA only samples).

FIG. 11 Hydrogel Gelation. Panel (a) shows a schematic representing the single step covalent crosslinking between tyrosine residues on silk and tyramine side chains on HA creating a composite hydrogel. Panel (b) shows images showing gelation of silk-HA hydrogels during a vial inversion test. Panel (c) shows gelation times, as determined by the vial inversion test, show hydrogels consisting of more than 1% HA decreased gelation time (n=4, ***p≤0.001). Panel (d) shows in addition, the increasing of HA concentration affected crosslinking kinetics by reducing the lag period seen most prominently in 0% hydrogels and decreasing time at which crosslinking was complete (n=7).

FIG. 12 Rheological Properties. Panel (a) shows representative time sweeps, panel (b) shows representative strain sweeps, and panel (c) shows final shear storage moduli. HA only hydrogels reached a final modulus much quicker (panel a), had a lower maximum strain (panel b), and had a much lower final storage modulus (panel c) as compared to composite and silk only hydrogels. (n=3, *p≤0.05, ***p≤0.001).

FIG. 13 Unconfined Compression. Panel (a) shows the compressive moduli of the hydrogels over time, expressed on a log scale, are dependent on HA concentration where increasing concentration reduces the amount the modulus increases after 1 month. Panel (b) represents average stress-strain curves and show that stress and hysteresis increase for all samples after 4 weeks, except for HA only hydrogels, with the extent of hysteresis reduced by increasing HA concentration. (n=5, +p≤0.05 compared to 0%, *p≤0.05, ***p≤0.001. Statistical analysis was performed after log transformation).

FIG. 14 FTIR Absorbance Spectra. Panel (a) shows the average FTIR absorbance spectrum in the amide I region is shown for hydrogels over time. Exemplary hydrogels with lower HA concentration exhibit a peak shift from ˜1640 cm⁻¹ to ˜1620 cm⁻¹ at 3 weeks. Additionally, the peak at ˜1620 cm⁻¹, which is representative of β-sheet formation, becomes larger and wider as HA concentration decreases. Panel (b) shows the ratio of β-sheet to random coil conformation and was calculated by dividing the average of peak absorbance at 1620-1625 and 1640-1650 cm⁻¹ showing that increasing HA concentration reduced the ratio over time. (n=5, *p≤0.05 and ***p≤0.001 compared to week 0).

FIG. 15 Percent Water. The percent water, based on mass, was calculated for hydrogels over time. Initially, hydrogels, with the exception of HA only hydrogels, contained ˜97% water. At weeks 1, 2, 3, and 4, composite hydrogels had higher percentages of water as compared to 0% hydrogels, showing that the addition of HA reduces the amount of water loss over time. (n=5, **p≤0.01, ***p≤0.001 compared to 0% HA).

FIG. 16 In vitro Degradation. Degradation was determined by calculating the fraction of initial mass after soaking in an enzymatic cocktail consisting of 1 U/mL and 0.001 U/mL of hyalruonidase and protease. A direct relation between degradation rate and HA concentration was seen when increasing HA concentration above 5% significantly increased the degradation rate at day 8 (n=4, **p≤0.01, ***p≤0.001).

FIG. 17 2D hMSC Response. Panel (a) shows bright-field images of 2D hMSCs at day 0 and 7. Hydrogels containing silk fibroin enhanced cell spreading whereas HA only hydrogels inhibited spreading (scale bar=100 μm). Panel (b) shows fold change of hMSC DNA content as compared to day 0 calculated for days 3, 5, and 7. Silk only and silk-HA (10% HA) hydrogels promoted cellular growth similar to that of tissue culture plastic (TCP) whereas HA only hydrogels showed inhibited growth (n=6, *p≤0.05, **p≤0.01, ***p≤0.001).

FIG. 18 Additional Rheological Properties. Representative frequency sweeps (0.01 to 100 rad/s at 1% strain) (panel a) and strain sweeps (0.1% to 500% or to failure, at 1 Hz) (panel b) show that the testing parameters selected are within the linear viscoelastic region (n=3).

FIG. 19 Reaction Mechanism. Schematic of the enzymatic polymerization of tyrosines in silk fibroin (red) and tyramines in hyaluronic acid (blue). Horseradish peroxidase (HPR) reacts with hydrogen peroxide (H₂O₂) to form a reactive intermediate (compound I), which then reacts with the phenolic group of tyrosine or tyramine to form a radical and a second HRP reactive intermediate (compound II). Additional phenolic radicals are formed via reaction of phenols with HRP compound I or II, where the reaction between a phenol and compound II returns the HRP to its resting state. These unstable phenolic radical can then react with one another to form dityrosine, dityramine, or tyrosine-tyramine covalent bonds.

FIG. 20 Crosslinking Efficiency. Exemplary crosslinking efficiency, as determined by percentage of polymer retained, in hydrogels was greater than 94%. Increasing HA concentration above 1% HA and 5% HA increased the crosslinking efficiency of silk fibroin and HA, respectively. (n=3, *p≤0.05, **p≤0.01, ***p<0.001).

FIG. 21 LC-MS. Panel (a) shows a schematic of the LC-MS spectra for each analyte that was seen in the hydrogels. Panel (b) shows hydrogels evaluated for the different crosslinks where + and − represent whether the crosslinks were or were not observed. (n=4).

FIG. 22 Crosslinking Kinetics. The intrinsic fluorescence of the phenolic crosslinks in silk only, silk-HA composite, and HA only hydrogels showed a peak at 415 nm after excitation at 315 nm (n=5) (panel a). From the crosslinking kinetics, the time to plateau was recorded (panel b), representing the time when crosslinking is complete. Increasing HA significantly decreased the time to plateau. (n=7, p≤0.05).

FIG. 23 hMSC Viability. Live/dead staining of 2D hMSCs at day 3 show that both silk and silk-HA (10% HA) hydrogels maintained cell viability similar to that of tissue culture plastic controls (TCP) whereas HA only hydrogels showed limited viable cells. (n=3, scale bar=100 μm, live cells and dead cells were stained green and red, respectively).

FIG. 24 hMSC Encapsulation. (a) Live/dead staining of encapsulated hMSCs at day 3 show that there was limited cell death and spread cell morphology in both silk and silk-HA (10% HA) hydrogels (n=3, scale bar=200 μm, live cells and dead cells were stained green and red, respectively). (b) AlamarBlue of the encapsulated cells at day 3 shows that silk only hydrogels exhibited lower overall metabolic activity when compared with silk-HA and HA only hydrogels (n=4, *p0.05, **p0.01).

FIG. 25 shows exemplary scanning electron microscope (SEM) images of exemplary provided hydrogels including 0%, 1%, 5%, 10%, or 20% HA both before and after in vitro degradation after 4 days. Also shown is an HA-only control.

FIG. 26 shows exemplary photographs of live/dead assay results of certain provided hydrogels encapsulating hMSCs after 2 weeks in culture without DMEM.

FIG. 27 shows exemplary photographs of certain provided compositions with hMSCs either encapsulated (top row), or seeded on the surface (bottom rows) after 3 days in culture and a CD44 stain applied.

FIG. 28 shows exemplary images of hMSCs stained with DAPI or Phalloidin 5 days after seeding on exemplary provided hydrogels.

FIG. 29 shows exemplary graphs of injection force testing on certain hydrogel compositions provided herein. Approximately 1 m: of hydrogel was injected through a 21 G thin-wall needle at 1 mm/s for 25 seconds.

FIG. 30 shows exemplary graphs of modulus and stress-strain of exemplary cervical tissue samples both before and after bulking with certain provided hydrogel compositions.

FIG. 31 shows a comparison of volumetric properties of exemplary cervical tissue samples both before and after bulking with certain provided hydrogel compositions. Also shown is an exemplary H&E stain showing the placement of the provided hydrogel compositions within the cervical tissue samples (bottom panel).

FIG. 32 shows exemplary images of live/dead stains of several exemplary provided hydrogel compositions including cervical fibroblast cells over 5 days of incubation.

FIG. 33 shows exemplary graphs of the metabolic activity and proliferation of cervical fibroblast cells on certain exemplary provided hydrogel compositions over 5 days of incubation.

FIG. 34 shows exemplary graphs of cytokine production of exemplary cervical fibroblast cells both before and after 24 hrs of stimulation with lipopolysaccharide (LPS).

### DEFINITIONS

In order for the present disclosure to be more readily understood, certain terms are first defined below. Additional definitions for the following terms and other terms are set forth throughout the specification.

In this application, unless otherwise clear from context, the term “a” may be understood to mean “at least one.” As used in this application, the term “or” may be understood to mean “and/or.” In this application, the terms “comprising” and “including” may be understood to encompass itemized components or steps whether presented by themselves or together with one or more additional components or steps. Unless otherwise stated, the terms “about” and “approximately” may be understood to permit standard variation as would be understood by those of ordinary skill in the art. Where ranges are provided herein, the endpoints are included. As used in this application, the term “comprise” and variations of the term, such as “comprising” and “comprises,” are not intended to exclude other additives, components, integers or steps.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Biocompatible”: The term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death (e.g., less than 10% or 5%), and/or their administration in vivo does not induce significant inflammation or other such adverse effects.

“Biodegradable”: As used herein, the term “biodegradable” refers to materials that, when introduced to cells (either internally or by being placed in proximity thereto), are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo. In some embodiments, biodegradable polymer materials break down into their component monomers. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

“Improve,” “increase”, “inhibit” or “reduce”: As used herein, the terms “improve”, “increase”, “inhibit”, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

“In vitro”: The term “in vitro” as used herein refers to events that occur in an artificial environment, e.g., in a test tube or reaction vessel, in cell culture, etc., rather than within a multi-cellular organism.

“In vivo”: as used herein refers to events that occur within a multi-cellular organism, such as a human and a non-human animal. In the context of cell-based systems, the term may be used to refer to events that occur within a living cell (as opposed to, for example, in vitro systems).

“Physiological conditions”: as used herein, has its art-understood meaning referencing conditions under which cells or organisms live and/or reproduce. In some embodiments, the term refers to conditions of the external or internal mileu that may occur in nature for an organism or cell system. In some embodiments, physiological conditions are those conditions present within the body of a human or non-human animal, especially those conditions present at and/or within a surgical site. Physiological conditions typically include, e.g., a temperature range of 20-40° C., atmospheric pressure of 1, pH of 6-8, glucose concentration of 1-20 mM, oxygen concentration at atmospheric levels, and gravity as it is encountered on earth. In some embodiments, conditions in a laboratory are manipulated and/or maintained at physiologic conditions. In some embodiments, physiological conditions are encountered in an organism.

“Polypeptide”: The term “polypeptide”, as used herein, generally has its art-recognized meaning of a polymer of at least two amino acids. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

“Protein”: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Proteins may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. In some embodiments, proteins are antibodies, antibody fragments, biologically active portions thereof, and/or characteristic portions thereof.

“Reference”: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

“Small molecule”: As used herein, the term “small molecule” is used to refer to molecules, whether naturally-occurring or artificially created (e.g., via chemical synthesis), having a relatively low molecular weight and being an organic and/or inorganic compound. Typically, a “small molecule” is monomeric and have a molecular weight of less than about 1500 g/mol. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not a protein or polypeptide (e.g., is not an oligopeptide or peptide). In some embodiments, a small molecule is not a polynucleotide (e.g., is not an oligonucleotide). In some embodiments, a small molecule is not a polysaccharide. In some embodiments, a small molecule does not comprise a polysaccharide (e.g., is not a glycoprotein, proteoglycan, glycolipid, etc.).

“Solution”: As used herein, the term “solution” broadly refers to a homogeneous mixture composed of one phase. Typically, a solution comprises a solute or solutes dissolved in a solvent or solvents. It is characterized in that the properties of the mixture (such as concentration, temperature, and density) can be uniformly distributed through the volume. In the context of the present application, therefore, a “silk fibroin solution” refers to silk fibroin protein in a soluble form, dissolved in a solvent, such as water. In some embodiments, silk fibroin solutions may be prepared from a solid-state silk fibroin material (i.e., silk matrices), such as silk films and other scaffolds. Typically, a solid-state silk fibroin material is reconstituted with an aqueous solution, such as water and a buffer, into a silk fibroin solution. It should be noted that liquid mixtures that are not homogeneous, e.g., colloids, suspensions, emulsions, are not considered solutions.

“Subject”: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

“Substantially”: As used herein, the term “substantially”, and grammatical equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

### Detailed Description of Certain Embodiments

As described herein, in some embodiments, provided compositions (e.g., hydrogels) are hydrophilic polymeric networks that can be utilized as scaffolds for biomedical applications including regenerative medicine, drug delivery, and tissue engineering. Due to their hydrophilic nature, provided compositions (e.g., hydrogels) are permeable to oxygen and nutrients and possess mechanics similar to that of native extracellular matrix, creating a receptive environment for cell proliferation. Additionally, the mechanics of the silk-HA composite hydrogels can be tuned for injectability and/or formed in situ, allowing for minimally invasive treatments when utilized in vivo.

Silk and Silk Fibroin

Silk is a natural protein fiber produced in a specialized gland of certain organisms. Silk production in organisms is especially common in the Hymenoptera (bees, wasps, and ants), and is sometimes used in nest construction. Other types of arthropod also produce silk, most notably various arachnids such as spiders (e.g., spider silk). Silk fibers generated by insects and spiders represent the strongest natural fibers known and rival even synthetic high performance fibers.

Silk has been a highly desired and widely used textile since its first appearance in ancient China (see Elisseeff, “The Silk Roads: Highways of Culture and Commerce,” Berghahn Books/UNESCO, New York (2000); see also Vainker, “Chinese Silk: A Cultural History,” Rutgers University Press, Piscataway, N.J. (2004)). Glossy and smooth, silk is favored by not only fashion designers but also tissue engineers because it is mechanically tough but degrades harmlessly inside the body, offering new opportunities as a highly robust and biocompatible material substrate (see Altman et al., Biomaterials, 24: 401 (2003); see also Sashina et al., Russ. J. Appl. Chem., 79: 869 (2006)).

Silk is naturally produced by various species, including, without limitation: Antheraea mylitta; Antheraea pernyi; Antheraea yamamai; Galleria mellonella; Bombyx mori; Bombyx mandarina; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis.

As is known in the art, silks are modular in design, with large internal repeats flanked by shorter (˜100 amino acid) terminal domains (N and C termini). Naturally-occurring silks have high molecular weight (200 to 350 kDa or higher) with transcripts of 10,000 base pairs and higher and >3000 amino acids (reviewed in Omenatto and Kaplan (2010) Science 329: 528-531). The larger modular domains are interrupted with relatively short spacers with hydrophobic charge groups in the case of silkworm silk. N- and C-termini are involved in the assembly and processing of silks, including pH control of assembly. The N- and C-termini are highly conserved, in spite of their relatively small size compared with the internal modules.

In accordance with various embodiments, silk fibroin may be modified prior to use in provided methods and compositions. For example, in some embodiments, silk fibroin may be modified to include more tyrosine groups than native silk fibroin (e.g., silk fibroin from a silkworm or a spider), or to include fewer tyrosine groups than native silk fibroin. By way of specific example, in some embodiments, silk fibroin may be modified to include at least one non-native tyrosine. In some embodiments, silk fibroin may be modified to remove or alter at least one tyrosine group from native silk fibroin such that it is no longer able to crosslink with a phenol-containing polymer. In some embodiments, addition of tyrosine groups to silk fibroin may occur via carbodiimide chemistry. Any known technique for quantifying the amount of tyrosine groups on silk fibroin may be used. For example, in some embodiments, quantification of tyrosine groups may be performing using one or more of spectrophotometric analysis (e.g., via UV absorbance), liquid chromatography-mass spectrometry (LC-MS), and high performance liquid chromatography (HPLC).

In general, silk fibroin for use in accordance with the present invention may be produced by any such organism, or may be prepared through an artificial process, for example, involving genetic engineering of cells or organisms to produce a silk protein and/or chemical synthesis. In some embodiments of the present invention, silk fibroin is produced by the silkworm, Bombyx mori. Fibroin is a type of structural protein produced by certain spider and insect species that produce silk. Cocoon silk produced by the silkworm, Bombyx mori, is of particular interest because it offers low-cost, bulk-scale production suitable for a number of commercial applications, such as textile.

Silkworm cocoon silk contains two structural proteins, the fibroin heavy chain (˜350 kDa) and the fibroin light chain (˜25 kDa), which are associated with a family of non-structural proteins termed sericin, which glue the fibroin brings together in forming the cocoon. The heavy and light chains of fibroin are linked by a disulfide bond at the C-terminus of the two subunits (see Takei, F., Kikuchi, Y., Kikuchi, A., Mizuno, S. and Shimura, K. (1987) 105 J. Cell Biol., 175-180; see also Tanaka, K., Mori, K. and Mizuno, S. 114 J. Biochem. (Tokyo), 1-4 (1993); Tanaka, K., Kajiyama, N., Ishikura, K., Waga, S., Kikuchi, A., Ohtomo, K., Takagi, T. and Mizuno, S., 1432 Biochim. Biophys. Acta., 92-103 (1999); Y Kikuchi, K Mori, S Suzuki, K Yamaguchi and S Mizuno, “Structure of the Bombyx mori fibroin light-chain-encoding gene: upstream sequence elements common to the light and heavy chain,” 110 Gene, 151-158 (1992)). The sericins are a high molecular weight, soluble glycoprotein constituent of silk which gives the stickiness to the material. These glycoproteins are hydrophilic and can be easily removed from cocoons by boiling in water.

As used herein, the term “silk fibroin” refers to silk fibroin protein, whether produced by silkworm, spider, or other insect, or otherwise generated (Lucas et al., 13 Adv. Protein Chem., 107-242 (1958)). In some embodiments, silk fibroin is obtained from a solution containing a dissolved silkworm silk or spider silk. For example, in some embodiments, silkworm silk fibroins are obtained, from the cocoon of Bombyx mori. In some embodiments, spider silk fibroins are obtained, for example, from Nephila clavipes. In the alternative, in some embodiments, silk fibroins suitable for use in the invention are obtained from a solution containing a genetically engineered silk harvested from bacteria, yeast, mammalian cells, transgenic animals or transgenic plants. See, e.g., WO 97/08315 and U.S. Pat. No. 5,245, 012, each of which is incorporated herein as reference in its entirety.

Thus, in some embodiments, a silk solution is used to fabricate compositions of the present invention contain fibroin proteins, essentially free of sericins. In some embodiments, silk solutions used to fabricate various compositions of the present invention contain the heavy chain of fibroin, but are essentially free of other proteins. In other embodiments, silk solutions used to fabricate various compositions of the present invention contain both the heavy and light chains of fibroin, but are essentially free of other proteins. In certain embodiments, silk solutions used to fabricate various compositions of the present invention comprise both a heavy and a light chain of silk fibroin; in some such embodiments, the heavy chain and the light chain of silk fibroin are linked via at least one disulfide bond. In some embodiments where the heavy and light chains of fibroin are present, they are linked via one, two, three or more disulfide bonds. Although different species of silk-producing organisms, and different types of silk, have different amino acid compositions, various fibroin proteins share certain structural features. A general trend in silk fibroin structure is a sequence of amino acids that is characterized by usually alternating glycine and alanine, or alanine alone. Such configuration allows fibroin molecules to self-assemble into a beta-sheet conformation. These “Alanine-rich” hydrophobic blocks are typically separated by segments of amino acids with bulky side-groups (e.g., hydrophilic spacers).

Silk materials explicitly exemplified herein were typically prepared from material spun by silkworm, Bombyx mori. Typically, cocoons are boiled in an aqueous solution of 0.02 M Na₂ CO₃, then rinsed thoroughly with water to extract the glue-like sericin proteins (this is also referred to as “degumming” silk). Extracted silk is then dissolved in a solvent, for example, LiBr (such as 9.3 M) solution at room temperature. A resulting silk fibroin solution can then be further processed for a variety of applications as described elsewhere herein.

In some embodiments, polymers of silk fibroin fragments can be derived by degumming silk cocoons at or close to (e.g., within 5% around) an atmospheric boiling temperature for at least about: 1 minute of boiling, 2 minutes of boiling, 3 minutes of boiling, 4 minutes of boiling, 5 minutes of boiling, 6 minutes of boiling, 7 minutes of boiling, 8 minutes of boiling, 9 minutes of boiling, 10 minutes of boiling, 11 minutes of boiling, 12 minutes of boiling, 13 minutes of boiling, 14 minutes of boiling, 15 minutes of boiling, 16 minutes of boiling, 17 minutes of boiling, 18 minutes of boiling, 19 minutes of boiling, 20 minutes of boiling, 25 minutes of boiling, 30 minutes of boiling, 35 minutes of boiling, 40 minutes of boiling, 45 minutes of boiling, 50 minutes of boiling, 55 minutes of boiling, 60 minutes or longer, including, e.g., at least 70 minutes, at least 80 minutes, at least 90 minutes, at least 100 minutes, at least 110 minutes, at least about 120 minutes or longer. As used herein, the term “atmospheric boiling temperature” refers to a temperature at which a liquid boils under atmospheric pressure.

In some embodiments, silk fibroin fragments may be of any application-appropriate size. For example, in some embodiments, silk fibroin fragments may have a molecular weight of 200 kDa or less (e.g., less than 125 kDa, 100 kDa, 75 kDa, 50 kDa). Without wishing to be held to a particular theory, it is contemplated that the size of silk fibroin fragments may impact gelation time and rate of crosslinking. By way of specific example, in some embodiments, use of silk fragments of a relatively low molecular weight (e.g., less than 200 kDa) may result in relatively more rapid crosslinking due, at least in part, to the greater mobility of the available chains for reacting in a crosslinking step.

In some embodiments, hydrogels of the present invention produced from silk fibroin fragments can be formed by degumming silk cocoons in an aqueous solution at temperatures of: about 30° C., about 35° C., about 40° C., about 45° C., about 50° C., about 45° C., about 60° C., about 65° C., about 70° C., about 75° C., about 80° C., about 85° C., about 90° C., about 95° C., about 100° C., about 105° C., about 110° C., about 115° C., about at least 120° C.

In some embodiments, silk fibroin fragments may be solubilized prior to gelation. In some embodiments, a carrier can be a solvent or dispersing medium. In some embodiments, a solvent and/or dispersing medium, for example, is water, cell culture medium, buffers (e.g., phosphate buffered saline), a buffered solution (e.g. PBS), polyol (for example, glycerol, propylene glycol, liquid polyethylene glycol, and the like), Dulbecco's Modified Eagle Medium, HEPES, Hank's balanced medium, Roswell Park Memorial Institute (RPMI) medium, fetal bovine serum, or suitable combinations and/or mixtures thereof.

In some embodiments, the properties of provided compositions may be modulated by controlling a concentration of silk fibroin. In some embodiments, a weight percentage of silk fibroin can be present in a solution at any concentration suited to a particular application. In some embodiments, an aqueous silk fibroin solution (or a provided composition, for example, a provided hydrogel) can have silk fibroin at a concentration of about 0.1 wt % to about 95 wt %, 0.1 wt % to about 75 wt %, or 0.1 wt % to about 50 wt %. In some embodiments, an aqueous silk fibroin solution (or a provided composition, for example, a provided hydrogel) can have silk fibroin at a concentration of about 0.1 wt % to about 10 wt %, about 0.1 wt % to about 5 wt %, about 0.1 wt % to about 2 wt %, or about 0.1 wt % to about 1 wt %. In some embodiments, a silk fibroin solution (or a provided composition, for example, a provided hydrogel) have silk fibroin at a concentration of about 10 wt % to about 50 wt %, about 20 wt % to about 50 wt %, about 25 wt % to about 50 wt %, or about 30 wt % to about 50 wt %. In some embodiments, a weight percent of silk in solution (or a provided composition, for example, a provided hydrogel) is about less than 1 wt %, is about less than 1.5 wt %, is about less than 2 wt %, is about less than 2.5 wt %, is about less than 3 wt %, is about less than 3.5 wt %, is about less than 4 wt %, is about less than 4.5 wt %, is about less than 5 wt %, is about less than 5.5 wt %, is about less than 6 wt %, is about less than 6.5 wt %, is about less than 7 wt %, is about less than 7.5 wt %, is about less than 8 wt %, is about less than 8.5 wt %, is about less than 9 wt %, is about less than 9.5 wt %, is about less than 10 wt %, is about less than 11 wt %, is about less than 12 wt %, is about less than 13 wt %, is about less than 14 wt %, is about less than 15 wt %, is about less than 16 wt %, is about less than 17 wt %, is about less than 18 wt %, is about less than 19 wt %, is about less than 20 wt %, is about less than 25 wt %, or is about less than 30 wt %.

In some embodiments, a provided composition is configured to be injectable. In some embodiments, a viscosity of an injectable composition is modified by using a pharmaceutically acceptable thickening agent. In some embodiments, a thickening agent, for example, is methylcellulose, xanthan gum, carboxymethyl cellulose, hydroxypropyl cellulose, carbomer, or combination thereof. A preferred concentration of the thickener depends upon a selected agent and viscosity for injection.

In some embodiments, a provided composition may form a porous matrix or scaffold (e.g., a foam, or lyophilized composition). For example, the porous scaffold can have a porosity of at least about 10%, at least about 20%, at least about 30%, at least about 40%, at least about 50%, at least about 60%, at least about 70%, at least about 80%, at least about 90%, or higher.

In certain embodiments, particularly where provided compositions are or comprise hygrogels, silk fibroin from Bombyx mori cocoons can provide protein that can be utilized as naturally derived hydrogels with good biocompatibility, mechanical strength and ease of chemical modifications. Silk-based hydrogels can be prepared via many methods including sonication, pH, vortexing, electric fields, polyols, surfactants, and enzymatic reactions. For example, as we previously reported, the covalently crosslinking of the phenolic groups of tyrosine residues in silk fibroin using horseradish peroxidase (HRP) and hydrogen peroxide (H₂O₂), resulted in a cytocompatible, mechanically tunable, elastomeric protein biomaterial. These enzymatically formed hydrogels can avoid the brittle nature of physically (beta sheet formed) crosslinked silk hydrogels. Due to their resilience, lack of harsh crosslinking conditions, and tunable mechanics, similar to that of native tissues, these enzymatically crosslinked silk hydrogels provide a useful system for efficient cell encapsulation and tissue engineering. However, these pure silk hydrogels lack bioactivity and depending on reaction conditions can undergo changes in mechanics over time due to slow crystallization.

Phenol-Containing Polymers/Peptides/Proteins

In some embodiments, any application-appropriate phenol-containing polymer may be used. The specific phenol-containing polymer(s) used in a particular embodiment may depend on one or more of: the specific application of a provided composition, the physical or mechanical properties desired in the resultant composition, or the desired time to gelation for a particular embodiment (e.g., if encapsulation of one or more active agents is desired, it may be advantageous to have rapid gelation occur, such within one minute or less from the initiation of a crosslinking step).

In some embodiments, a phenol-containing polymer is or comprises a peptide or protein. In some embodiments, a phenol-containing polymer is a hydrophilic and/or bioactive polymer. In some embodiments, a phenol-containing polymer is or comprises a tyramine-containing and/or tyrosine-containing peptide or protein. In some embodiments, a tyramine-containing and/or tyrosine-containing peptide or protein is a peptide or protein that has been modified to incorporate one or more tyramine and/or tyrosine groups such that they are available to react as described herein. In some embodiments, a tyramine-containing and/or tyrosine-containing polymer is or comprises hyaluronic acid and/or polyethylene glycol.

Due to its biological and structural importance, as well as its ease of modification, HA can be an attractive polymer in general for biomedical applications and has been explored in drug delivery, synovial fluid supplementation, ocular and anti-adhesive surgery aids, wound healing, and soft tissue repair and augmentation. Since the turnover of HA is rapid (about ⅓ of the total HA body content is degraded and reformed daily), covalent crosslinking is necessary to increase mechanical stability for tissue engineering purposes. Covalent crosslinking can occur either directly through chemical approaches or by modifying the hydroxyl or carboxyl groups of HA with functional moieties, which can then be crosslinked. Tyramine-substituted HA (TS-HA) has been previously synthesized to provide a biocompatible hydrogel that can be enzymatically crosslinked, avoiding the harsh environment often required for chemical crosslinking methods. By utilizing HRP and H₂O₂, the tyramine functionalized carboxyl groups are covalently linked allowing for hydrogel formation under physiological conditions. These hydrogels have been explored in a wide range of applications in tissue engineering and drug delivery.

The combination of silk and hyaluronic acid has been previously explored in the form of hydrogels, films, microparticles, and sponges for cartilage, neuronal, and cardiac tissue engineering. One method of combining these two polymers in a bulk hydrogel is entrapping hyaluronic acid in a crystalline silk network through the application of ultrasonication. This physically crosslinked hydrogel facilitated human mesenchymal (hMSC) attachment and growth while providing mechanical integrity. A similar method was used to develop silk fibroin/HA hydrogels, which provided adequate mechanics and biological cues to maintain nucleus pulposus-like chondrogenic cell growth for tissue regeneration. However, the reliance on physically crosslinked, β-sheet, networks, results in brittle behavior, limiting applications and making them difficult to handle.

In some embodiments, a tyramine-containing and/or tyrosine-containing polymer comprises a modified form (e.g., wherein one or more phenol or tyramine group(s) added) of one or more of: dopamine, L-DOPA, serotonin, adrenaline, noradrenaline, salicylic acid, alginate, dextran, collagen, gelatin, chitosan, carboxymethylcellulose, heparin, poly(vinyl alcohol), sugars (e.g., lactose, cellulose, mannose, galactose, glucose, maltose, etc) or dimers or trimers thereof.

It is specifically contemplated that any of a variety of amounts of phenol-containing polymer may be used in accordance with various embodiments. By way of specific non-limiting example, in some embodiments, provided compositions may include between 0.01 wt % and 75 wt % phenol-containing polymer. In some embodiments, provided compositions may include at least 0.01 wt % phenol-containing polymer (e.g., at least 0.05, 0.1, 0.5 1, 2, 3, 4, 5, 10, 15, 20, 25, 30, 35, 40 45, 50, 55, 60, 65 wt %). In some embodiments, provided compositions may include at most 75 wt % phenol-containing polymer (e.g., at most 70, 65, 60, 55, 50, 45, 40, 35, 30, 25, 20, 15, 10, 5 wt %).

Crosslinking

Prior to the present invention, there were few reports that address modulating the enzymatically crosslinked silk hydrogels through crosslinking of polymers, peptides, and proteins utilizing this enzymatic reaction. Recently, the enzymatic crosslinking of silk and cardiac extracellular matrix (cECM) showed enhanced growth in vitro and infiltration in vivo but did not show a large effect in stiffening over time (see Stoppel, W. et al., 2016, “Elastic, silk-cardiac extracellular matrix hydrogels exhibit time-dependent stiffening that modulates cardiac cell response.” Journal of Biomedical Materials Research: Part A., 104(12): 3058-3072). To overcome these factors, we sought to incorporate hydrophilic, bioactive molecules to potentially inhibit crystallization and stiffening and provide cell instructive cues. To this end, in some embodiments, we incorporated hyaluronic acid (HA) or hyaluronan. HA is a naturally occurring, non-sulfated glucosaminoglycan, that consists of repeating disaccharide units of D-glucuronic acid and N-acetyl-D-glucosamine, linked by β-1-3 and β-1-4 glycosidic bonds. HA is ubiquitous throughout the body, but is highly localized in the extracellular matrix (ECM) of connective tissue, synovial joint fluid, vitreous humor, and brain tissue. HA is highly hydrophilic and has a net negative charge, providing tissues with hydration and structural support. In addition, HA, depending on molecular weight, plays an important role in many biological functions such as embryonic development, inflammation, angiogenesis, cell-matrix interactions, and wound healing.

As is known in the art, the process of joining two or more peptide/protein molecules through intermolecular covalent bonds is referred to crosslinking. A variety of crosslinking modes are contemplated as useful in accordance with various embodiments. Notably, in some embodiments, the methods of crosslinking described herein avoid harsh crosslinking conditions including, but not limited to—use of harsh or toxic chemicals and/or use of physical crosslinking (e.g., β-sheet formation). In some embodiments, a crosslinking step may occur in an aqueous environment. In some embodiments, a crosslinking step may occur in the absence of organic solvents or other toxic materials.

Covalently crosslinking, for example, silk and hyaluronic acid hydrogels using a variety of chemical crosslinking methods has been proposed to treat skin and soft tissue conditions. Common chemical crosslinkers, such as butanediol diglycidyl ether (BDDE), operate under harsh conditions, not only leading to HA degradation but also prohibiting cell encapsulation. A milder method of crosslinking that was described involved the use of 1-ethyl-3-[3-dimethylaminopropyl]carbodiimide hydrochloride (EDC) chemistry with hexamethylene diamine (HDMass.). Although this method can be carried out under physiological conditions, the crosslinking agents can be cytotoxic. Therefore hydrogels created using these methods are often washed prior to injection and can possibly lead to cell and tissue death when crosslinked in situ. Accordingly, in some embodiments, harsh or toxic reagent, such as BDDE, EDC, and/or HMDA are not used.

Accordingly, as described herein, we copolymerized silk and TS-HA using an enzymatic crosslinking step to generate biocompatible, elastomeric hydrogels that have both the mechanical integrity of silk and the structural and biological properties of hyaluronic acid. Additionally, the incorporation of the hydrophilic HA chains maintains local hydration, delaying and in some cases preventing crystallization and stiffening. By modulating the polymer, HRP and H₂O₂ concentrations, the mechanical properties, crystallization, and biological responses over extended times could be controlled. Ultimately, this hydrogel will provide a versatile tunable platform for a wide range of biomedical applications including cell encapsulation, tissue regeneration, and tissue augmentation. One of skill in the art will appreciate that this technology is not limited to the crosslinking of silk and TS-HA but can also be used to with any polymer/peptide/protein containing phenolic groups to incorporate specific biological or mechanical properties.

In some embodiments, silk fibroin and a phenol-containing polymer are crosslinked directly to one another (e.g., there is no spacer between the two, for example, no epoxide or multiamine spacer). In some embodiments, crosslinking in provided methods and compositions is or comprises multi-phenol crosslinks. In some embodiments, multi-phenol crosslinks are selected from the group consisting of di-tyrosine crosslinks, di-tyramine crosslinks, and tyrosine-tyramine crosslinks. In some embodiments, crosslinking does not include physical crosslinking (e.g., via β-sheet formation). In some embodiments wherein the crosslinking does not include physical crosslinking, provided compositions or methods may include β-sheet formation during a later time or step subsequent to a crosslinking step. In some embodiments, crosslinking does not include chemical crosslinking (e.g., using harsh or toxic chemicals, via addition reaction(s), exposure to high energy radiation such as gamma rays or electron beams). In some embodiments, crosslinking may comprise one or more of a condensation reaction(s), carbodiimide crosslinking, and glutaraldehyde crosslinking.

In some aspects, provided methods may comprise contacting a silk solution with an enzyme, and inducing gelation of the silk solution comprising the enzyme in the presence of a substrate for the enzyme. In some embodiments, the mixture can be mixed gently to induce gelation. In some embodiments, the method employs a horseradish peroxidase enzyme and hydrogen peroxide to enzymatically crosslink silk fibroins. Without wishing to be bound by theory, the horseradish peroxidase enzyme and hydrogen peroxide (e.g., an oxidizing agent) can be used to enzymatically crosslink the tyrosine side chains that are found in the native silk fibroin. The gel initiation and gelation rate and/or kinetic properties of the process can be tunable or controlled, for example, depending on concentrations of silk, enzyme (e.g., HRP), and/or substrate for the enzyme (e.g., H₂O₂).

In some embodiments, the silk fibroin and phenol-containing polymer may each be provided in a separate solution prior to the associating step. Without wishing to be held to a particular theory, in some embodiments, attempting to solubilize silk fibroin with at least one phenol-containing polymer may result in aggregation and/or precipitation of the silk and/or inability to fully solubilize the phenol-containing polymer.

Previous methods often resulted in crosslinking agents remaining in association with a crosslinked composition, requiring some sort of purification or removal step. In some embodiments, provided methods avoid this need. Accordingly, in some embodiments, provided methods do not include a purification step. In some embodiments, provided methods do not require a removal step to reduce or eliminate the presence of one or more contaminants (e.g., cross linkers, toxic or harsh chemicals, etc) before use (e.g., injection or other administration). In some embodiments, provided methods do not require or utilize organic solvents (particularly not volatile organic solvents).

Crosslinking-Enzymes

In accordance with various embodiments, provided methods include the use of one or more enzymes, along with an appropriate exogenous substrate, if needed, capable of forming covalent bonds between silk fibroin and at least one phenol-containing polymer (e.g., forming covalent bonds directly between silk fibroin and a phenol-containing polymer). In accordance with various embodiments where a provided composition is or comprises a hydrogel, provided methods may include one or more steps to induce gelation including gentle mixing, heating, etc as appropriate for a particular enzyme (and potentially substrate). In some embodiments, enzymatic crosslinking (e.g., as is used in many embodiments of gelation reactions herein) is induced by addition of an enzyme substrate, e.g., before, after, or together with the enzyme. In some embodiments, phenolic groups on a phenol-containing polymer are the substrate for an enzyme used in provided methods. By way of specific example, in some embodiments, when an enzyme (e.g., horseradish peroxidase) is combined with H₂O₂ in the presence of phenolic groups, the horseradish peroxidase will catalyze the decomposition of H₂O₂ at the expense of aromatic proton donors (i.e. phenol) in a phenol-containing polymer. As such, for example, in some embodiments, silk fibroin (which contain tyrosines) and other phenol-containing polymers may act as a reducing substrate. In such cases, the reaction ultimately results in phenolic radicals that can form a covalent bonds via condensation of aromatic rings.

In accordance with various embodiments, any of a variety of enzymes may be used to crosslink silk fibroin with one or more phenol-containing polymers. In some embodiments, the enzyme is or comprises a peroxidase. In some embodiments, a peroxidase may be or comprise a plant-based or mammal-based peroxidase. In some embodiments, an enzyme or substrate may be or comprise at least one of hydrogen peroxide, tyrosinase, laccase, hemin, a microperoxidase, cytochrome c, porphyrins, fenton, soy bean peroxidase, myeloperoxidase, lactoperoxidase, eosinophil peroxidase, thyroid peroxidase, prostaglandin H synthase, and horseradish peroxidase. In some embodiments an enzyme substrate is a peroxide. In some embodiments, a peroxide is hydrogen peroxide, barium peroxide, calcium peroxide, sodium peroxide, organic peroxides or combinations thereof.

In some embodiments, methods of providing, preparing, and/or manufacturing a covalently crosslinked hydrogel in accordance with the present invention comprises enzymatically introducing crosslinks. In some embodiments, a method of providing, preparing, and/or manufacturing a covalently crosslinked hydrogel in accordance with the present invention comprises introducing crosslinks with peroxidase (e.g., in the presence of peroxide). In some embodiments, a peroxidase selected from the group consisting of animal heme-dependent peroxidase, bromoperoxidase, glutathione peroxidase, haloperoxidase, horseradish peroxidase, lactoperoxidase, myeloperoxidase, thyroid peroxidase, vanadium and combinations thereof. In some embodiments, a peroxidase is utilized at a concentration between about 0.001 mg/mL and about 10 mg/mL. In some embodiments, a peroxide is selected from the group consisting of barium peroxide, calcium peroxide, hydrogen peroxide, sodium peroxide, organic peroxides and combinations thereof.

In some embodiments, provided compositions (e.g., hydrogels) of the present invention may be provided, prepared, and/or manufactured from a solution of protein polymer (e.g., of silk such as silk fibroin) that is adjusted to (e.g., by dialysis) and/or maintained at a sub-physiological pH (e.g., at or below a pH significantly under pH 7). For example, in some embodiments, a provided composition is provided, prepared, and/or manufactured from a solution of protein polymer that is adjusted to and/or maintained at a pH near or below about 6. In some embodiments, a provided composition is provided, prepared, and/or manufactured from a solution of protein polymer with a pH for instance about 6 or less, or about 5 or less. In some embodiments, a provided composition is provided, prepared, and/or manufactured from a solution of protein polymer with a pH in a range for example of at least 6, at least 7, at least 8, at least 9, and at least about 10.

In some embodiments, an enzyme (e.g., a peroxidase) is utilized in a concentration between, for example: about 0.001 mg/mL and about 100 mg/mL, about 0.001 mg/mL and about 90 mg/mL, about 0.001 mg/mL and about 80 mg/mL, about 0.001 mg/mL and about 70 mg/mL, about 0.001 mg/mL and about 60 mg/mL, about 0.001 mg/mL and about 50 mg/mL, about 0.001 mg/mL and about 40 mg/mL, about 0.001 mg/mL and about 30 mg/mL, about 0.001 mg/mL and about 20 mg/mL, about 0.001 mg/mL and about 10 mg/mL, or about 0.001 mg/mL and about 5 mg/mL. In some embodiments, a solution concentration, for example a peroxidase concentration is: less than about 1 mg/mL, less than about 1.5 mg/mL, less than about 2 mg/mL, less than about 2.5 mg/mL, less than about 3 mg/mL, less than about 3.5 mg/mL, less than about 4 mg/mL, less than about 4.5 mg/mL, less than about 5 mg/mL, less than about 5.5 mg/mL, less than about 6 mg/mL, less than about 6.5 mg/mL, less than about 7 mg/mL, less than about 7.5 mg/mL, less than about 8 mg/mL, less than about 8.5 mg/mL, less than about 9 mg/mL, less than about 9.5 mg/mL, less than about 10 mg/mL, less than about 11 mg/mL, less than about 12 mg/mL, less than about 13 mg/mL, less than about 14 mg/mL, less than about 15 mg/mL, less than about 16 mg/mL, less than about 17 mg/mL, less than about 18 mg/mL, less than about 19 mg/mL, or less than about 20 mg/mL.

In some embodiments, one or more agents or enhancers may be used in accordance with provided methods. By way of specific example, in some embodiments wherein horseradish peroxidase is used, hydrogen peroxide (H₂O₂) may also be included, for example, as an oxidizing agent. In some embodiments, an oxidizing agent or enhancer (e.g., hydrogen peroxide) may have a concentration between 0.1 and 100 mM (e.g., between 1 and 100 mM, 10 and 100 mM, 1 and 50 mM, etc).

Exemplary Properties of Compositions

As described herein, including in the Examples below, provided methods allow for the creation of compositions having any of a variety of enhanced properties.

Typically, a composition (e.g., hydrogel) is formed in vitro for later use. In contrast, some embodiments of provided methods may be formed in situ. Without wishing to be held to a particular theory, this ability of some embodiments may be due, at least in part, to the mild crosslinking conditions used herein. In some embodiments, provided compositions may be formed during administration or immediately before administration.

In some embodiments, provided methods and compositions include a very rapid time to gelation. Specifically, in accordance with various embodiments, provided compositions may exhibit any of a range of gelation times. For example, where a slower gelation time is desired for a particular application, a provided composition may be tuned to gel over a period of approximately 30 minutes to two hours (e.g., between 30 minutes and one hour, between 30 minutes and 90 minutes, or between one hour and two hours). For example, where a slower gelation time is desired for a particular application, a provided composition may be tuned to gel over a period of approximately 30 minutes or less (e.g., 25 minutes, 20 minutes, 15 minutes, 10 minutes, 5 minutes, 1 minute, or less). In some embodiments, a provided hydrogel exhibits a gelation time of between 10 seconds and 20 minutes after the crosslinking step (e.g., between 10 seconds and 60 seconds, between 10 seconds and 30 seconds, between 10 second and 20 seconds). In some embodiments, increasing the relative amount of phenol-containing polymer results in a decreased time to gelation.

In some embodiments, provided compositions may exhibit improved storage modulus and/or higher strain to failure characteristics. By way of specific example, in some embodiments, provided compositions may exhibit a strain to failure of at least 20% (e.g. at least 30%, 40%, 50%, 60%, 70%, 80%, 90%, 100%, 125%, 150%, or more), while previously known compositions exhibit a strain to failure of at most 10%. In some embodiments, provided compositions may have a storage modulus between 10 Pa-5.5 KPa (e.g., between 10 Pa and 5 KPa, between 10 Pa and 1 KPa, between 100 Pa and 5.5 KPa, between 100 Pa and 1 KPa, etc).

In some embodiments, provided compositions may exhibit improved unconfined compressive properties (e.g., compressive modulus). In some embodiments, provided compositions exhibit an increase of 100% or more in the compressive modulus, as compared to compositions not including crosslinking of silk fibroin and a phenol-containing polymer as herein described). By way of additional example, in some embodiments, a provided composition may have a compressive moduli of between 200 Pa and 1 MPa (e.g., between 200 Pa and 500 kPa).

In some embodiments, provided compositions may exhibit improved or altered types of crosslinks as compared to compositions (e.g., gels) created using prior methods. For example, in some embodiments, provided compositions may include primarily (e.g., greater than 50% of the total crosslinks) dityramine crosslinks or tyramine-tyrosine crosslinks. In some embodiments, a provided composition may include substantially only (e.g., greater than 95% of the total crosslinks) dityramine crosslinks. In some embodiments, a provided composition may include substantially only (e.g., greater than 95% of the total crosslinks) tyramine-tyrosine crosslinks.

In some embodiments, provided compositions may exhibit a lower degree of crystallization over time (e.g. beta-sheet crystallization). In some embodiments, provided compositions exhibit substantially no crystallization (e.g. beta-sheet crystallization) over a particular time frame, for example, a week, a month, 3 months, six months, or a year or more. In some embodiments, the amount of crystallization over time may be assessed via FTIR analysis, specifically, by quantifying a shift in the spectra from 1640 cm⁻¹ to 1620 cm⁻¹. In some embodiments, the shift may be quantified, for example, by determining the ratio of the average peak absorbance at 1620-1625 cm⁻¹ and 1640-1650 cm⁻¹ which represents the ratio of silk fibroin in beta-sheet configuration as compared that the silk fibroin in random coil configuration. Generally, an increasing ratio means that there is increasing beta-sheet content as compared to the amount of random coil present in a particular composition. In some embodiments, FTIR spectra may be deconvoluted by fitting a Gaussian curve. In some embodiments, the degree of crystallization in a particular composition may be assessed via x-ray scattering and/or circular dichroism.

In some embodiments, provided compositions may exhibit improved swelling properties. Specifically, in some embodiments, provided compositions may exhibit a mass fraction of at most 1.00 after soaking in an aqueous solution for 12 hours. In some embodiments, provided compositions may have mass fractions of between 0.4-0.99 after soaking in an aqueous solution for 12 hours. In some embodiments, provided compositions may have mass fractions of greater than 1.00 (e.g., greater than 1.1, 1.2. 1.3, 1.4, 1.5, 2.0, 2.5, 5.0, etc) after soaking in an aqueous solution for 12 hours. In some embodiments, provided compositions may exhibit a mass fraction of between 0.4 and 5.0 after soaking in an aqueous solution for 12 hours.

Active Agents

In some embodiments, provided compositions (e.g., hydrogels) can comprise one or more (e.g., one, two, three, four, five or more) active agents and/or functional moieties (together, “additives”). Without wishing to be bound by a theory, an additive can provide or enhance one or more desirable properties, e.g., strength, flexibility, ease of processing and handling, biocompatibility, bioresorability, surface morphology, release rates and/or kinetics of one or more active agents present in the composition, and the like. In some embodiments, one or more such additives can be covalently or non-covalently linked with a composition (e.g., with a polymer such as silk fibroin that makes up the hydrogel) and can be integrated homogenously or heterogeneously (e.g., in a gradient or in discrete portions of a provided composition) within the silk composition.

In some embodiments, an additive is or comprises a moiety covalently associated (e.g., via chemical modification or genetic engineering) with a polymer (e.g., silk fibroin or a phenol-containing polymer). In some embodiments, an additive is non-covalently associated with a hydrogel or hydrogel component.

In some embodiments, provided compositions (e.g., hydrogels) comprise additives at a total amount from about 0.01 wt % to about 99 wt %, from about 0.01 wt % to about 70 wt %, from about 5 wt % to about 60 wt %, from about 10 wt % to about 50 wt %, from about 15 wt % to about 45 wt %, or from about 20 wt % to about 40 wt %, of the total silk composition. In some embodiments, ratio of silk fibroin to additive in the composition can range from about 1000:1 (w/w) to about 1:1000 (w/w), from about 500:1 (w/w) to about 1:500 (w/w), from about 250:1 (w/w) to about 1:250 (w/w), from about 200:1 (w/w) to about 1:200 (w/w), from about 25:1 (w/w) to about 1:25 (w/w), from about 20:1 (w/w) to about 1:20 (w/w), from about 10:1 (w/w) to about 1:10 (w/w), or from about 5:1 (w/w) to about 1:5 (w/w).

In some embodiments, provided compositions (e.g., hydrogels) include one or more additives at a molar ratio relative to polymer (i.e., a polymer:additive ratio) of, e.g., at least 1000:1, at least 900:1, at least 800:1, at least 700:1, at least 600:1, at least 500:1, at least 400:1, at least 300:1, at least 200:1, at least 100:1, at least 90:1, at least 80:1, at least 70:1, at least 60:1, at least 50:1, at least 40:1, at least 30:1, at least 20:1, at least 10:1, at least 7:1, at least 5:1, at least 3:1, at least 1:1, at least 1:3, at least 1:5, at least 1:7, at least 1:10, at least 1:20, at least 1:30, at least 1:40, at least 1:50, at least 1:60, at least 1:70, at least 1:80, at least 1:90, at least 1:100, at least 1:200, at least 1:300, at least 1:400, at least 1:500, at least 600, at least 1:700, at least 1:800, at least 1:900, or at least 1:100.

In some embodiments, moiety polymer:additive ratio is, e.g., at most 1000:1, at most 900:1, at most 800:1, at most 700:1, at most 600:1, at most 500:1, at most 400:1, at most 300:1, at most 200:1, 100:1, at most 90:1, at most 80:1, at most 70:1, at most 60:1, at most 50:1, at most 40:1, at most 30:1, at most 20:1, at most 10:1, at most 7:1, at most 5:1, at most 3:1, at most 1:1, at most 1:3, at most 1:5, at most 1:7, at most 1:10, at most 1:20, at most 1:30, at most 1:40, at most 1:50, at most 1:60, at most 1:70, at most 1:80, at most 1:90, at most 1:100, at most 1:200, at most 1:300, at most 1:400, at most 1:500, at most 1:600, at most 1:700, at most 1:800, at most 1:900, or at most 1:1000.

In some embodiments, moiety polymer:additive ratio is, e.g., from about 1000:1 to about 1:1000, from about 900:1 to about 1:900, from about 800:1 to about 1:800, from about 700:1 to about 1:700, from about 600:1 to about 1:600, from about 500:1 to about 1:500, from about 400:1 to about 1:400, from about 300:1 to about 1:300, from about 200:1 to about 1:200, from about 100:1 to about 1:100, from about 90:1 to about 1:90, from about 80:1 to about 1:80, from about 70:1 to about 1:70, from about 60:1 to about 1:60, from about 50:1 to about 1:50, from about 40:1 to about 1:40, from about 30:1 to about 1:30, from about 20:1 to about 1:20, from about 10:1 to about 1:10, from about 7:1 to about 1:7, from about 5:1 to about 1:5, from about 3:1 to about 1:3, or about 1:1.

In some embodiments, provided compositions (e.g., hydrogels) comprise additives, for example, therapeutic, preventative, and/or diagnostic agents.

In some embodiments, an additive is or comprises one or more therapeutic agents. In general, a therapeutic agent is or comprises a small molecule and/or organic compound with pharmaceutical activity (e.g., activity that has been demonstrated with statistical significance in one or more relevant pre-clinical models or clinical settings). In some embodiments, a therapeutic agent is a clinically-used drug. In some embodiments, a therapeutic agent is or comprises an cells, proteins, peptides, nucleic acid analogues, nucleotides, oligonucleotides, nucleic acids (DNA, RNA, siRNA), peptide nucleic acids, aptamers, antibodies or fragments or portions thereof, anesthetic, anticoagulant, anti-cancer agent, inhibitor of an enzyme, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, anti-glaucoma agent, neuroprotectant, angiogenesis inhibitor, hormones, hormone antagonists, growth factors or recombinant growth factors and fragments and variants thereof, cytokines, enzymes, antibiotics or antimicrobial compounds, antifungals, antivirals, toxins, prodrugs, chemotherapeutic agents, small molecules, drugs (e.g., drugs, dyes, amino acids, vitamins, antioxidants), pharmacologic agents, and combinations thereof.

In some embodiments, provided compositions (e.g., hydrogels) comprise additives, for example, cells. In some embodiments, methods of using provided compositions may comprise adhering cells to a surface of a covalently crosslinked hydrogel. In some embodiments, methods of using provided compositions may comprise encapsulating cells within a matrix a covalently crosslinked hydrogel. In some embodiments, methods of using provided compositions may comprise encapsulating cells for introducing cells to a native tissue. Cells suitable for use herein include, but are not limited to, progenitor cells or stem cells, smooth muscle cells, skeletal muscle cells, cardiac muscle cells, glial cells (e.g., astrocytes), neurons, epithelial cells, endothelial cells, urothelial cells, fibroblasts, myoblasts, chondrocytes, chondroblasts, osteoblasts, osteoclasts, keratinocytes, hepatocytes, bile duct cells, pancreatic islet cells, thyroid, parathyroid, adrenal, hypothalamic, pituitary, ovarian, testicular, salivary gland cells, adipocytes, and precursor cells.

In some embodiments, provided compositions (e.g., hydrogels) comprise additives, for example, organisms, such as, a bacterium, fungus, plant or animal, or a virus. In some embodiments, an active agent may include or be selected from neurotransmitters, hormones, intracellular signal transduction agents, pharmaceutically active agents, toxic agents, agricultural chemicals, chemical toxins, biological toxins, microbes, and animal cells such as neurons, liver cells, and immune system cells. The active agents may also include therapeutic compounds, such as pharmacological materials, vitamins, sedatives, hypnotics, prostaglandins and radiopharmaceuticals.

In some embodiments, provided compositions (e.g., hydrogels) comprise additives, for example, antibiotics. Antibiotics suitable for incorporation in various embodiments include, but are not limited to, aminoglycosides (e.g., neomycin), ansamycins, carbacephem, carbapenems, cephalosporins (e.g., cefazolin, cefaclor, cefditoren, cefditoren, ceftobiprole), glycopeptides (e.g., vancomycin), macrolides (e.g., erythromycin, azithromycin), monobactams, penicillins (e.g., amoxicillin, ampicillin, cloxacillin, dicloxacillin, flucloxacillin), polypeptides (e.g., bacitracin, polymyxin B), quinolones (e.g., ciprofloxacin, enoxacin, gatifloxacin, ofloxacin, etc.), sulfonamides (e.g., sulfasalazine, trimethoprim, trimethoprim-sulfamethoxazole (co-trimoxazole)), tetracyclines (e.g., doxycyline, minocycline, tetracycline, etc.), chloramphenicol, lincomycin, clindamycin, ethambutol, mupirocin, metronidazole, pyrazinamide, thiamphenicol, rifampicin, thiamphenicl, dapsone, clofazimine, quinupristin, metronidazole, linezolid, isoniazid, fosfomycin, fusidic acid, β-lactam antibiotics, rifamycins, novobiocin, fusidate sodium, capreomycin, colistimethate, gramicidin, doxycycline, erythromycin, nalidixic acid, and vancomycin. For example, β-lactam antibiotics can be aziocillin, aztreonam, carbenicillin, cefoperazone, ceftriaxone, cephaloridine, cephalothin, moxalactam, piperacillin, ticarcillin and combination thereof.

In some embodiments, provided compositions (e.g., hydrogels) comprise additives, for example, anti-inflammatories. Anti-inflammatory agents may include corticosteroids (e.g., glucocorticoids), cycloplegics, non-steroidal anti-inflammatory drugs (NSAIDs), immune selective anti-inflammatory derivatives (ImSAIDs), and any combination thereof. Exemplary NSAIDs include, but not limited to, celecoxib (Celebrex®); rofecoxib (Vioxx®), etoricoxib (Arcoxia®), meloxicam (Mobic®), valdecoxib, diclofenac (Voltaren®, Cataflam®), etodolac (Lodine®), sulindac (Clinori®), aspirin, alclofenac, fenclofenac, diflunisal (Dolobid®), benorylate, fosfosal, salicylic acid including acetylsalicylic acid, sodium acetylsalicylic acid, calcium acetylsalicylic acid, and sodium salicylate; ibuprofen (Motrin), ketoprofen, carprofen, fenbufen, flurbiprofen, oxaprozin, suprofen, triaprofenic acid, fenoprofen, indoprofen, piroprofen, flufenamic, mefenamic, meclofenamic, niflumic, salsalate, rolmerin, fentiazac, tilomisole, oxyphenbutazone, phenylbutazone, apazone, feprazone, sudoxicam, isoxicam, tenoxicam, piroxicam (Feldene®), indomethacin (Indocin®), nabumetone (Relafen®), naproxen (Naprosyn®), tolmetin, lumiracoxib, parecoxib, licofelone (ML3000), including pharmaceutically acceptable salts, isomers, enantiomers, derivatives, prodrugs, crystal polymorphs, amorphous modifications, co-crystals and combinations thereof.

In some embodiments, provided compositions (e.g., hydrogels) comprise additives, for example, antibodies. Suitable antibodies for incorporation in hydrogels include, but are not limited to, abciximab, adalimumab, alemtuzumab, basiliximab, bevacizumab, cetuximab, certolizumab pegol, daclizumab, eculizumab, efalizumab, gemtuzumab, ibritumomab tiuxetan, infliximab, muromonab-CD3, natalizumab, ofatumumab omalizumab, palivizumab, panitumumab, ranibizumab, rituximab, tositumomab, trastuzumab, altumomab pentetate, arcitumomab, atlizumab, bectumomab, belimumab, besilesomab, biciromab, canakinumab, capromab pendetide, catumaxomab, denosumab, edrecolomab, efungumab, ertumaxomab, etaracizumab, fanolesomab, fontolizumab, gemtuzumab ozogamicin, golimumab, igovomab, imciromab, lab etuzumab, mepolizumab, motavizumab, nimotuzumab, nofetumomab merpentan, oregovomab, pemtumomab, pertuzumab, rovelizumab, ruplizumab, sulesomab, tacatuzumab tetraxetan, tefibazumab, tocilizumab, ustekinumab, visilizumab, votumumab, zalutumumab, and zanolimumab.

In some embodiments, provided compositions (e.g., hydrogels) comprise additives, for example, polypeptides (e.g., proteins), including but are not limited to: one or more antigens, cytokines, hormones, chemokines, enzymes, and any combination thereof as an agent and/or functional group. Exemplary enzymes suitable for use herein include, but are not limited to, peroxidase, lipase, amylose, organophosphate dehydrogenase, ligases, restriction endonucleases, ribonucleases, DNA polymerases, glucose oxidase, laccase, and the like.

In some embodiments, provided compositions (e.g., hydrogels) comprise additives, for example, particularly useful for wound healing. In some embodiments, agents useful for wound healing include stimulators, enhancers or positive mediators of the wound healing cascade (e.g., wound healing growth factors) which 1) promote or accelerate the natural wound healing process or 2) reduce effects associated with improper or delayed wound healing, which effects include, for example, adverse inflammation, epithelialization, angiogenesis and matrix deposition, and scarring and fibrosis.

In some embodiments, provided compositions (e.g., hydrogels) comprise additives, for example, an optically or electrically active agent, including but not limited to, chromophores; light emitting organic compounds such as luciferin, carotenes; light emitting inorganic compounds, such as chemical dyes; light harvesting compounds such as chlorophyll, bacteriorhodopsin, protorhodopsin, and porphyrins; light capturing complexes such as phycobiliproteins; and related electronically active compounds; and combinations thereof.

Nucleic Acids

In some embodiments, provided compositions (e.g., hydrogels) comprise additives, for example, nucleic acid agents. In some embodiments, a composition may release nucleic acid agents. In some embodiments, a nucleic acid agent is or comprises a therapeutic agent. In some embodiments, a nucleic acid agent is or comprises a diagnostic agent. In some embodiments, a nucleic acid agent is or comprises a prophylactic agent.

It will be appreciated by those of ordinary skill in the art that a nucleic acid agent can have a length within a broad range. In some embodiments, a nucleic acid agent has a nucleotide sequence of at least about 40, for example at least about 60, at least about 80, at least about 100, at least about 200, at least about 500, at least about 1000, or at least about 3000 nucleotides in length. In some embodiments, a nucleic acid agent has a length from about 6 to about 40 nucleotides. For example, a nucleic acid agent may be from about 12 to about 35 nucleotides in length, from about 12 to about 20 nucleotides in length or from about 18 to about 32 nucleotides in length.

In some embodiments, nucleic acid agents may be or comprise deoxyribonucleic acids (DNA), ribonucleic acids (RNA), peptide nucleic acids (PNA), morpholino nucleic acids, locked nucleic acids (LNA), glycol nucleic acids (GNA), threose nucleic acids (TNA), and/or combinations thereof.

In some embodiments, a nucleic acid has a nucleotide sequence that is or comprises at least one protein-coding element. In some embodiments, a nucleic acid has a nucleotide sequence that is or comprises at least one element that is a complement to a protein-coding sequence. In some embodiments, a nucleic acid has a nucleotide sequence that includes one or more gene expression regulatory elements (e.g., promoter elements, enhancer elements, splice donor sites, splice acceptor sites, transcription termination sequences, translation initiation sequences, translation termination sequences, etc.). In some embodiments, a nucleic acid has a nucleotide sequence that includes an origin of replication. In some embodiments, a nucleic acid has a nucleotide sequence that includes one or more integration sequences. In some embodiments, a nucleic acid has a nucleotide sequence that includes one or more elements that participate in intra- or inter-molecular recombination (e.g., homologous recombination). In some embodiments, a nucleic acid has enzymatic activity. In some embodiments, a nucleic acid hybridizes with a target in a cell, tissue, or organism. In some embodiments, a nucleic acid acts on (e.g., binds with, cleaves, etc.) a target inside a cell. In some embodiments, a nucleic acid is expressed in a cell after release from a provided composition. In some embodiments, a nucleic acid integrates into a genome in a cell after release from a provided composition.

In some embodiments, nucleic acid agents have single-stranded nucleotide sequences. In some embodiments, nucleic acid agents have nucleotide sequences that fold into higher order structures (e.g., double and/or triple-stranded structures). In some embodiments, a nucleic acid agent is or comprises an oligonucleotide. In some embodiments, a nucleic acid agent is or comprises an antisense oligonucleotide. Nucleic acid agents may include a chemical modification at the individual nucleotide level or at the oligonucleotide backbone level, or it may have no modifications.

In some embodiments of the present invention, a nucleic acid agent is an siRNA agent. Short interfering RNA (siRNA) comprises an RNA duplex that is approximately 19 basepairs long and optionally further comprises one or two single-stranded overhangs. An siRNA may be formed from two RNA molecules that hybridize together, or may alternatively be generated from a single RNA molecule that includes a self-hybridizing portion. It is generally preferred that free 5′ ends of siRNA molecules have phosphate groups, and free 3′ ends have hydroxyl groups. The duplex portion of an siRNA may, but typically does not, contain one or more bulges consisting of one or more unpaired nucleotides. One strand of an siRNA includes a portion that hybridizes with a target transcript. In certain preferred embodiments of the invention, one strand of the siRNA is precisely complementary with a region of the target transcript, meaning that the siRNA hybridizes to the target transcript without a single mismatch. In other embodiments of the invention one or more mismatches between the siRNA and the targeted portion of the target transcript may exist. In most embodiments of the invention in which perfect complementarity is not achieved, it is generally preferred that any mismatches be located at or near the siRNA termini.

Short hairpin RNA refers to an RNA molecule comprising at least two complementary portions hybridized or capable of hybridizing to form a double-stranded (duplex) structure sufficiently long to mediate RNAi (typically at least 19 base pairs in length), and at least one single-stranded portion, typically between approximately 1 and 10 nucleotides in length that forms a loop. The duplex portion may, but typically does not, contain one or more bulges consisting of one or more unpaired nucleotides. As described further below, shRNAs are thought to be processed into siRNAs by the conserved cellular RNAi machinery. Thus shRNAs are precursors of siRNAs and are, in general, similarly capable of inhibiting expression of a target transcript.

In describing siRNAs it will frequently be convenient to refer to sense and antisense strands of the siRNA. In general, the sequence of the duplex portion of the sense strand of the siRNA is substantially identical to the targeted portion of the target transcript, while the antisense strand of the siRNA is substantially complementary to the target transcript in this region as discussed further below. Although shRNAs contain a single RNA molecule that self-hybridizes, it will be appreciated that the resulting duplex structure may be considered to comprise sense and antisense strands or portions. It will therefore be convenient herein to refer to sense and antisense strands, or sense and antisense portions, of an shRNA, where the antisense strand or portion is that segment of the molecule that forms or is capable of forming a duplex and is substantially complementary to the targeted portion of the target transcript, and the sense strand or portion is that segment of the molecule that forms or is capable of forming a duplex and is substantially identical in sequence to the targeted portion of the target transcript.

For purposes of description, the discussion below may refer to siRNA rather than to siRNA or shRNA. However, as will be evident to one of ordinary skill in the art, teachings relevant to the sense and antisense strand of an siRNA are generally applicable to the sense and antisense portions of the stem portion of a corresponding shRNA. Thus in general the considerations below apply also to shRNAs.

An siRNA agent is considered to be targeted to a target transcript for the purposes described herein if 1) the stability of the target transcript is reduced in the presence of the siRNA or shRNA as compared with its absence; and/or 2) the siRNA or shRNA shows at least about 90%, more preferably at least about 91%, 92%, 93%, 94%, 95%, 96%, 97%, 98%, 99%, or 100% precise sequence complementarity with the target transcript for a stretch of at least about 15, more preferably at least about 17, yet more preferably at least about 18 or 19 to about 21-23 nucleotides; and/or 3) one strand of the siRNA or one of the self-complementary portions of the shRNA hybridizes to the target transcript under stringent conditions for hybridization of small (<50 nucleotide) RNA molecules in vitro and/or under conditions typically found within the cytoplasm or nucleus of mammalian cells. Since the effect of targeting a transcript is to reduce or inhibit expression of the gene that directs synthesis of the transcript, an siRNA, shRNA, targeted to a transcript is also considered to target the gene that directs synthesis of the transcript even though the gene itself (i.e., genomic DNA) is not thought to interact with the siRNA, shRNA, or components of the cellular silencing machinery. Thus in some embodiments, an siRNA, shRNA, that targets a transcript is understood to target the gene that provides a template for synthesis of the transcript.

In some embodiments, an siRNA agent can inhibit expression of a polypeptide (e.g., a protein). Exemplary polypeptides include, but are not limited to, matrix metallopeptidase 9 (MMP-9), neutral endopeptidase (NEP) and protein tyrosine phosphatase 1B (PTP1B).

Growth Factors

In some embodiments, provided compositions (e.g., hydrogels) comprise additives, for example, one or more growth factors. In some embodiments, a provided composition may release one or more growth factors. In some embodiments, a provided composition may release multiple growth factors. In some embodiments growth factors known in the art include, for example, adrenomedullin, angiopoietin, autocrine motility factor, basophils, brain-derived neurotrophic factor, bone morphogenetic protein, colony-stimulating factors, connective tissue growth factor, endothelial cells, epidermal growth factor, erythropoietin, fibroblast growth factor, fibroblasts, glial cell line-derived neurotrophic factor, granulocyte colony stimulating factor, granulocyte macrophage colony stimulating factor, growth differentiation factor-9, hepatocyte growth factor, hepatoma-derived growth factor, insulin-like growth factor, interleukins, keratinocyte growth factor, keratinocytes, lymphocytes, macrophages, mast cells, myostatin, nerve growth factor, neurotrophins, platelet-derived growth factor, placenta growth factor, osteoblasts, platelets, proinflammatory, stromal cells, T-lymphocytes, thrombopoietin, transforming growth factor alpha, transforming growth factor beta, tumor necrosis factor-alpha, vascular endothelial growth factor and combinations thereof.

In some embodiments, provided compositions (e.g., hydrogels) comprise additives, for example, that are particularly useful for healing. Exemplary agents useful as growth factors for defect repair and/or healing can include, but are not limited to, growth factors for defect treatment modalities now known in the art or later-developed; exemplary factors, agents or modalities including natural or synthetic growth factors, cytokines, or modulators thereof to promote bone and/or tissue defect healing. Suitable examples may include, but not limited to 1) topical or dressing and related therapies and debriding agents (such as, for example, Santyl® collagenase) and Iodosorb® (cadexomer iodine); 2) antimicrobial agents, including systemic or topical creams or gels, including, for example, silver-containing agents such as SAGs (silver antimicrobial gels), (CollaGUARD™, Innocoll, Inc) (purified type-I collagen protein based dressing), CollaGUARD Ag (a collagen-based bioactive dressing impregnated with silver for infected wounds or wounds at risk of infection), DermaSIL™ (a collagen-synthetic foam composite dressing for deep and heavily exuding wounds); 3) cell therapy or bioengineered skin, skin substitutes, and skin equivalents, including, for example, Dermograft (3-dimensional matrix cultivation of human fibroblasts that secrete cytokines and growth factors), Apligraf® (human keratinocytes and fibroblasts), Graftskin® (bilayer of epidermal cells and fibroblasts that is histologically similar to normal skin and produces growth factors similar to those produced by normal skin), TransCyte (a Human Fibroblast Derived Temporary Skin Substitute) and Oasis® (an active biomaterial that comprises both growth factors and extracellular matrix components such as collagen, proteoglycans, and glycosaminoglycans); 4) cytokines, growth factors or hormones (both natural and synthetic) introduced to the wound to promote wound healing, including, for example, NGF, NT3, BDGF, integrins, plasmin, semaphoring, blood-derived growth factor, keratinocyte growth factor, tissue growth factor, TGF-alpha, TGF-beta, PDGF (one or more of the three subtypes may be used: AA, AB, and B), PDGF-BB, TGF-beta 3, factors that modulate the relative levels of TGFβ3, TGFβ1, and TGFβ2 (e.g., Mannose-6-phosphate), sex steroids, including for example, estrogen, estradiol, or an oestrogen receptor agonist selected from the group consisting of ethinyloestradiol, dienoestrol, mestranol, oestradiol, oestriol, a conjugated oestrogen, piperazine oestrone sulphate, stilboestrol, fosfesterol tetrasodium, polyestradiol phosphate, tibolone, a phytoestrogen, 17-beta-estradiol; thymic hormones such as Thymosin-beta-4, EGF, HB-EGF, fibroblast growth factors (e.g., FGF1, FGF2, FGF7), keratinocyte growth factor, TNF, interleukins family of inflammatory response modulators such as, for example, IL-10, IL-1, IL-2, IL-6, IL-8, and IL-10 and modulators thereof; INFs (INF-alpha, -beta, and -delta); stimulators of activin or inhibin, and inhibitors of interferon gamma prostaglandin E2 (PGE2) and of mediators of the adenosine 3′,5′-cyclic monophosphate (cAMP) pathway; adenosine Al agonist, adenosine A2 agonist or 5) other agents useful for wound healing, including, for example, both natural or synthetic homologues, agonist and antagonist of VEGF, VEGFA, IGF; IGF-1, proinflammatory cytokines, GM-CSF, and leptins and 6) IGF-1 and KGF cDNA, autologous platelet gel, hypochlorous acid (Sterilox® lipoic acid, nitric oxide synthase3, matrix metalloproteinase 9 (MMP-9), CCT-ETA, alphavbeta6 integrin, growth factor-primed fibroblasts and Decorin, silver containing wound dressings, Xenaderm™, papain wound debriding agents, lactoferrin, substance P, collagen, and silver-ORC, placental alkaline phosphatase or placental growth factor, modulators of hedgehog signaling, modulators of cholesterol synthesis pathway, and APC (Activated Protein C), keratinocyte growth factor, TNF, Thromboxane A2, NGF, BMP bone morphogenetic protein, CTGF (connective tissue growth factor), wound healing chemokines, decorin, modulators of lactate induced neovascularization, cod liver oil, placental alkaline phosphatase or placental growth factor, and thymosin beta 4. In certain embodiments, one, two three, four, five or six agents useful for wound healing may be used in combination. More details can be found in U.S. Pat. No. 8,247,384, the contents of which are incorporated herein by reference.

It is to be understood that growth factor agents useful for healing (including for example, growth factors and cytokines) above encompass all naturally occurring polymorphs (for example, polymorphs of the growth factors or cytokines). Also, functional fragments, chimeric proteins comprising one of said agents useful for wound healing or a functional fragment thereof, homologues obtained by analogous substitution of one or more amino acids of the wound healing agent, and species homologues are encompassed. It is contemplated that one or more agents useful for wound healing may be a product of recombinant DNA technology, and one or more agents useful for wound healing may be a product of transgenic technology. For example, platelet derived growth factor may be provided in the form of a recombinant PDGF or a gene therapy vector comprising a coding sequence for PDGF.

In some embodiments, provided compositions (e.g., hydrogels) comprise additives, for example, that are particularly useful as diagnostic agents. In some embodiments, diagnostic agents include gases; commercially available imaging agents used in positron emissions tomography (PET), computer assisted tomography (CAT), single photon emission computerized tomography, x-ray, fluoroscopy, and magnetic resonance imaging (MM); and contrast agents. Examples of suitable materials for use as contrast agents in MRI include gadolinium chelates, as well as iron, magnesium, manganese, copper, and chromium. Examples of materials useful for CAT and x-ray imaging include iodine-based materials.

In some embodiments, provided compositions (e.g., hydrogels) comprise additives, for example, that are or comprise fluorescent and/or luminescent moieties. Fluorescent and luminescent moieties include a variety of different organic or inorganic small molecules commonly referred to as “dyes,” “labels,” or “indicators.” Examples include fluorescein, rhodamine, acridine dyes, Alexa dyes, cyanine dyes, etc. Fluorescent and luminescent moieties may include a variety of naturally occurring proteins and derivatives thereof, e.g., genetically engineered variants. For example, fluorescent proteins include green fluorescent protein (GFP), enhanced GFP, red, blue, yellow, cyan, and sapphire fluorescent proteins, reef coral fluorescent protein, etc. Luminescent proteins include luciferase, aequorin and derivatives thereof. Numerous fluorescent and luminescent dyes and proteins are known in the art (see, e.g., U.S. Patent Application Publication No.: 2004/0067503; Valeur, B., “Molecular Fluorescence: Principles and Applications,” John Wiley and Sons, 2002; Handbook of Fluorescent Probes and Research Products, Molecular Probes, 9^{th} edition, 2002; and The Handbook—A Guide to Fluorescent Probes and Labeling Technologies, Invitrogen, 10^{th} edition, available at the Invitrogen web site; both of which are incorporated herein by reference).

Forms of Composition and Additional Structures

In accordance with various embodiments, provided compositions may take any of several forms. In some embodiments, a provided composition may be or comprise a tube, particle, film, foam, wire, hydrogel, etc. In some embodiments, a provided composition may be or comprise a lyophilized form of a tube, particle, film, foam, wire, etc. In some embodiments, a provided composition may be or comprise a hydrogel. In some embodiments, a provided composition may further include an additional structure such as a tube, particle, film, foam, wire, hydrogel, etc. In some embodiments, a provided composition may be partially or totally encapsulated in an additional structure. In some embodiments, a provided composition may partially or totally encapsulate an additional structure.

As will be recognized by one of skill in the art, previously known hydrogel compositions are typically made from synthetic and natural polymers, for example, polyesters, polyurethanes, polyethers, elastin, resilin. Synthetic polymers have also been developed that exhibit high resilience and recovery from both applied tensile and compressive forces. Poly(glycerol sebacate) (PGS) for example has shown utility as a scaffold for engineering vascular, cardiac, and nerve tissues. Additionally, synthetic bioelastomers based on polyurethanes, including for examples variants of poly(ethylene glycol), poly(c-caprolactone), and poly(vinyl alcohol), modified with degradable segments have also been developed and used for soft tissue, bone, and myocardial repairs. The present disclosure encompasses the recognition of significant drawback, however, that are often associated with traditional hydrogels, both natural and synthetic. For example, although desirable features such as tunable mechanics, cell encapsulation attributes, biocompatibility, biodegradability or elasticity have been reported for certain traditional hydrogels, the present disclosure appreciates that, in general, such traditional hydrogels cannot offer a combination of all of these characteristics.

By way of specific example, previously developed hydrogel technologies typically lack certain of the mechanical properties described for hydrogels herein, and/or lack the ability to specifically tune such properties, e.g., via production methodologies. Alternatively or additionally, previously developed hydrogel technologies typically lack certain of the favorable degradation mechanics provided by hydrogels described herein and/or lack the ability to specifically tune such properties. Still further, in many cases, traditional hydrogels fail to display certain degradation properties described for hydrogels provided herein; rapid degradation of such previously-developed hydrogels often limits their use to short term scaffolding. Yet further, in many cases, traditional hydrogel technologies require organic solvents during processing, which can result in toxicity, can interfere with cell or protein encapsulation (and particularly with maintenance of structural and/or functional integrity of encapsulated entities), and cannot resist long term strains when incorporated in vivo.

Further, many traditional hydrogels form, at least in part, through physical entanglements and hydrogen bonding between hydrophobic domains, resulting in β-sheet formation. β-sheet crystals have been shown to provide structure, strength, and long term stability of hydrogels. However, β-sheet crystals also display brittle behavior, as the crystals prevent long range displacements. Accordingly, and as described herein, provided compositions, including those in hydrogel form, provide sophisticated control and/or balance of such properties.

Exemplary Uses

In accordance with various embodiments, provided compositions may be suitable for use in any of a variety of methods including methods of treating, methods of forming, and others. In some embodiments, provided compositions may be useful in the areas including, but not limited to, cell encapsulation, drug delivery, cell delivery, tissue regeneration (e.g., muscle, cardiac, cartilage, neural), and soft tissue augmentation (e.g., as a soft tissue filler, or component thereof). In some embodiments, provided compositions may be useful as in vitro models, or portions thereof.

By way of additional example, in some embodiments, provided compositions may be used as adhesives. For example, in some embodiments, provided compositions can adhere to a surface, e.g., a rough surface, a smooth surface, a porous surface, a non-porous or substantially non-porous surface, and/or surfaces made of specific materials, for example, metal surfaces, ceramic surfaces, organic surfaces (e.g. biological tissue), etc.

Routes of Administration

Provided compositions may be administered via any application appropriate route or via any application appropriate manner. To give but a few non-limiting examples, exemplary modes of administration to a subject include, but are not limited to, topical, implant, injection, infusion, spray, instillation, implantation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

Certain provided examples below show, inter alia, that, in some embodiments, increasing hyaluronic acid concentration in provided compositions decreases gel stiffening and crystallization over time. In various embodiments, these new hydrogels may be useful for any of a variety of applications including, but not limited to, cell encapsulation, in drug delivery and for soft tissue augmentation. Also provided herein are data showing that covalently crosslinking a tyramine-conjugated polymer to silk fibroin through enzymatic means can be used to control hydrogel properties such mechanics, crystallinity, and biological response. In some embodiments, this system is versatile in that other phenol-conjugated polymers, proteins, and/or peptides can easily be crosslinked into the silk hydrogel to obtain desired traits.

These examples, in part, characterized the properties of certain exemplary provided single step, enzymatically crosslinked composite hydrogels consisting of silk and tyramine-substituted hyaluronic acid. It was found that, in some embodiments, the increasing hyaluronic acid content within the hybrid hydrogels decreased gelation time and helped to reduce the changes in mechanics and crystallization seen over time. Overall, the present invention provides, according to various embodiments, a composite hydrogel platform in tissue engineering that can provide a tunable, more stable system with the mechanical integrity of silk and the bioactivity of hyaluronic acid.

### Example 1

### Materials and Methods for Preparation of Silk-HA Hydrogels and Determination of Material Characteristics

In the present study, enzymatically crosslinked silk-based hydrogels were explored by investigating the effects of covalently binding HA to the silk.

Aqueous silk solutions were prepared using our previously established methods. Briefly, sericin protein was removed from Bombyx mori silkworm cocoons by placing 5 g of cut cocoons in 2 L of a boiling 0.02 M sodium carbonate solution (Sigma-Aldrich, St. Louis, Mo.) for 30 or 60 minutes. After rinsing in deionized water three times, the degummed fibers were dried overnight and solubilized in 9.3 M LiBr (Sigma-Aldrich, St. Louis, Mo.) for 4 hours at 60° C. The resulting silk solution was then dialyzed against deionized (DI) water using standard grade regenerated cellulose dialysis tubing (3.5 kD MWCO, Spectrum Labs Inc, Rancho Dominguez, Calif.). After 6 changes over 3 days, insoluble silk particulates were removed by centrifugation (two times at 9000 RPM, 5° C., 20 minutes). Silk concentration was determined by weighing dried solution of a known volume. A final silk solution of 4% w/v was obtained by diluting with DI water unless otherwise noted.

Lyophilized TS-HA (CORGEL® powder 2.8% unless otherwise noted, MW 0.9-1 MDa, Lifecore Biomedical, Chaska, Minn.) was dissolved in Ultrapure™ distilled water (ThermoFisher Scientific, Waltham, Mass.) overnight at 4° C. Aqueous silk and TS-HA were combined through gentle pipetting to yield a final silk concentration of 2% w/v and final TS-HA concentrations of 0.5, 1, 5, 10, 20, 30% w/w (weight of HA/total weight) (Table 1). Hydrogels consisting of silk or TS-HA only contained the same silk or TS-HA concentration. Crosslinking was initiated by adding of 10 U/mL of horseradish peroxidase (HRP, type VI, 1000 U/mL stock, Sigma-Aldrich, St. Louis, Mo.) followed by 0.1% v/v H₂O₂ (1% v/v stock, Sigma-Aldrich, St. Louis, Mo.). After pipet mixing, the solution was allowed to gel for 3-4 hours at 37° C. prior to analysis, unless otherwise noted.

TABLE 1
Sample Conditions. Samples are denoted by the mass percent of
TS-HA as compared to the total polymer concentration. Each condition
was crosslinked with 10 U/mL of HRP and 0.1% v/v H₂O₂.

| | Silk Concentration | TS-HA Concentration |
|---|---|---|
| Sample Name | (mg/mL) | (mg/mL) |
| 0% HA | 20 | 0 |
| 0.5% HA | 20 | 0.10 |
| 1% HA | 20 | 0.20 |
| 5% HA | 20 | 1.05 |
| 10% HA | 20 | 2.22 |
| 20% HA | 20 | 5.00 |
| 30% HA | 20 | 8.57 |
| 0.5% HA only | 0 | 0.10 |
| 1% HA only | 0 | 0.20 |
| 5% HA only | 0 | 1.05 |
| 10% HA only | 0 | 2.22 |
| 20% HA only | 0 | 5.00 |
| 30% HA only | 0 | 8.57 |

Gelation Kinetics and Rheology. Gelation time was evaluated by a vial inversion test, where gel point was designated as the time at which the solution no longer flowed after tilting of the vials. Briefly, H₂O₂ was added into a 60 minute degummed silk-(TS-HA) solution containing HRP in a 7 mL scintillation vial and mixed with a pipette for 3 seconds. Samples were incubated at 37° C. and evaluated every 2 minutes. Di-tyrosine crosslinking kinetics were evaluated through fluorescence spectroscopy using a SpectraMax M2 multi-mode microplate reader (Molecular Devices, Sunnyvale, Calif.). 200 μL of silk-(TS-HA) solution was mixed in a clear bottom 96-well plate for 3 seconds with a pipette. Di-tyrosine formation was monitored by measuring the intensity of fluorescence emission at 415 nm after excitation at 315 nm for 2 hours at 37° C. Results were reported as a fraction of the maximum intensity after subtracting a blank measurement taken prior to adding H₂O₂. Gelation time and kinetics through the inversion test and fluorescence spectroscopy were also determined for samples diluted with PBS to yield a final concentration of 0.5× PBS instead of Ultrapure™ water.

Rheological measurements were performed on a TA Instruments ARES-LS2 rheometer (TA Instruments, New Castle, Del.) using a 25 mm stainless steel upper cone and bottom plate. Samples were mixed as previously mentioned, without the addition of H₂O₂ and using 30 minute degummed silk. Silk-(TS-HA) solution with HRP was loaded onto the plate and the cone was lowered to the specified gap. During a 10 second precycle at a steady shear rate of 100/sec, H₂O₂ was added into the gap. To prevent evaporation during analysis, mineral oil was placed around the system. A dynamic time sweep was performed at 1 Hz with a 1% applied strain until samples attained a steady modulus. Following gelation, dynamic frequency sweeps (0.1 to 100 rad/s at 1% strain) and strain sweeps (0.1% to 500% or to failure, whichever came first, at 1 Hz) were conducted.

Liquid Chromatography and Mass Spectroscopy (LC-MS). Relative compositions of covalent bonds were analyzed using an Agilent 1200 series LC in tandem with an Agilent 6410 triple-quadruple MS (Agilent Technologies, Santa Clara, Calif.) operated in positive electrospray ionization mode. Preformed gels made with 30 minute degummed silk and 5% substituted TS-HA were hydrolyzed in 6N HCl (Sigma-Aldrich, Saint Louis, Mo.) with 0.1% w/v phenol (Invitrogen, Carlsbad, Calif.) for 4 hours at 120° C. After drying at 50° C. for 12 hrs, the dehydrated samples were reconstituted in 75% v/v acetonitrile in water (both LC/MS grade, Fisher Scientific, Waltham, Mass.) and diluted by 5x. Samples were then passed through a 0.2 μm PTFE filter and 200 ul of sample was added to a 96-well plate. Twenty microliter samples were injected into a hydrophobic interaction liquid chromatography column (Zorbax HILIC Plus, 4.6 mm×100 mm, 3.5 μm, Agilent Technologies, Santa Clara, Calif.) at 40° C. and a rate of 1.0 μL/min. The samples were first equilibrated at 95:5 acetonitrile (0.1% v/v formic acid) to water (0.1%v/v formic acid). After 1 min the mobile phase was adjusted to 5:95 acetonitrile to water over 5 minutes, held at high water for 2 min, and then returned to high acetonitrile. Agilent Mass Hunter software was run in scan mode between 200 m/z and 1000 m/z. The nitrogen gas was operated at a flow rate of 11 L/min and set to 300° C. Analytes with their molecular weight and m/z can be seen in Table 2.

TABLE 2
Analyte Description. Analytes that were determined through
LC-MS were tyrosine, dityrosine, tyramine, dityramine,
and tyrosine-tyramine. Crosslinked analytes can be either
single or double protonated, leading to 2 m/z values.

| Analyte | MW | m/z [H + 1] | m/z [H + 2] |
|---|---|---|---|
| Tyrosine | 181.1 | 182.1 | — |
| Dityrosine | 360.1 | 361.1 | 181.1 |
| Tyramine | 137.1 | 138.1 | — |
| Dityramine | 272.2 | 273.2 | 137.1 |
| Tyrosine- | 316.2 | 317.2 | 159.1 |
| tyramine | | | |

Unconfined compression. Unconfined compression testing was performed on a TA Instruments RSA3 Dynamic Mechanical Analyzer (TA Instruments, New Castle, Del.) between stainless steel parallel plates. Preformed hydrogels made with 30 minute degummed silk were placed under a preload of ˜0.5 g to ensure full contact. Two load-unload cycles to 30% strain at a rate of 0.667% per second were performed to eliminate artifacts. Stress response and elastic recovery were monitored during the third load-unload cycle at the same strain rate. A subset of hydrogels were tested initially and after incubation in 1× PBS at 37° C. at week 1, week 2, week 3, and week 4. Tangent modulus was calculated for each sample between 5 and 10% strain.

Fourier Transform Infrared Spectroscopy (FTIR). The secondary structure was analyzed by a JASCO FTIR 6200 spectrometer (JASCO, Tokyo, Japan) with a MIRacle™ attenuated total reflection with germanium crystal. Preformed hydrogels made with 30 minute degummed silk were washed in deuterated water (Sigma-Aldrich, St. Louis, Mo.) three times for 30 minutes each prior to analysis to remove the interference of water in the amide I region. Data was obtained by averaging 32 scans with a resolution of 4 cm⁻¹ within the wavenumber range of 600 and 4000 cm⁻¹. Samples were initially analyzed, allowed to incubate in 1× PBS at 37° C., and then analyzed at weeks 1, 2, 3, and 4. Data is shown as the fraction of maximum intensity after background spectra was subtracted.

Swelling and Opacity. Swelling was assessed by determining the fraction of initial mass after incubation at 37° C. in 1 × PBS and water at 1, 3, 6, and 12 hours. Mass was determined after hydrogels made with 60 minute degummed silk were blotted with a WypAll paper towel (Kimberly-Clark Co., Neenah, Wis.) to remove residual surface water.

To monitor the opacity overtime, 200 μL of the hydrogels made with 30 minute degummed silk were placed in a clear 96-well plate. After 4 hours, once gelation was complete, the opacity was quantified by measuring the absorbance at 550 nm using a microplate reader. Hydrogels were incubated in 1× PBS at 37° C. and absorbance was measured at weeks 1, 2, 3, and 4.

Statistics: Data are expressed as mean ±standard deviations. One- or Two-way ANOVA (analysis of variance) with Tukey's post hoc multiple comparison tests were utilized to determine statistically significant differences (*p<0.05, **p<0.01, ***p<0.001).

### Example 2

### Gelation Kinetics and Rheology

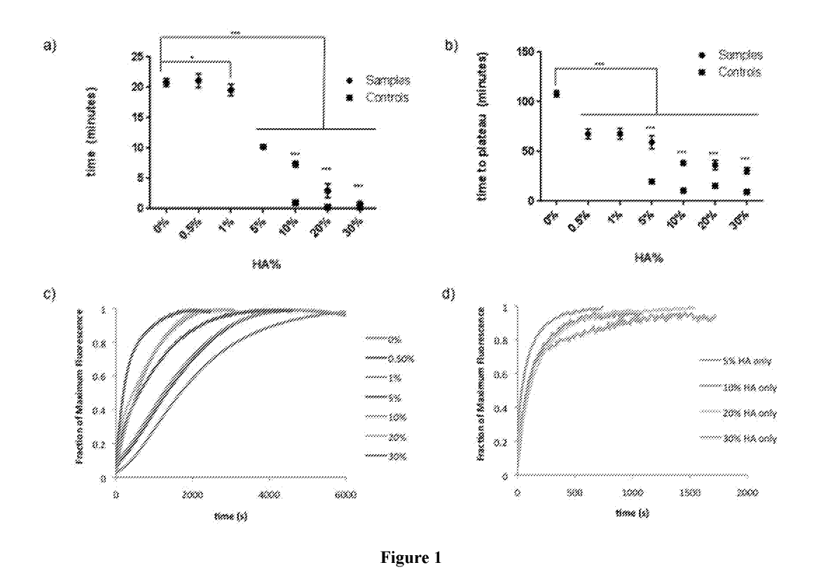

Gelation time, also known as the sol-gel transition, was determined through a vial inversion test where the times at which the solution no longer flowed after being tilted were recorded (FIG. 1, panel a). Hydrogels with HA concentration greater than 1% decreased gelation time significantly as compared with hydrogels consisting of 0% HA (p<0.001). Hyaluronic acid only, control samples that formed solid gels (10%, 20%, and 30% HA only samples) gelled within 15 seconds after H₂O₂ was added to the solution. Controls containing less than 2.22 mg/mL (0.5%, 1%, and 5% HA only) did not form solid gels.

The crosslinking kinetics were assessed by quantifying the rate of formation of di-tyrosine which has an intrinsic fluorescence that emits at 415 nm when excited at 315 nm. Both the shape of the curve and the time to plateau were dependent on HA content (FIG. 1, panels b-d). Samples containing lower than 5% HA experienced a delay at earlier time point with the 0% HA sample being most prominent. Controls with HA only completed crosslinking much faster than that of the silk only or silk-HA hybrid hydrogels.

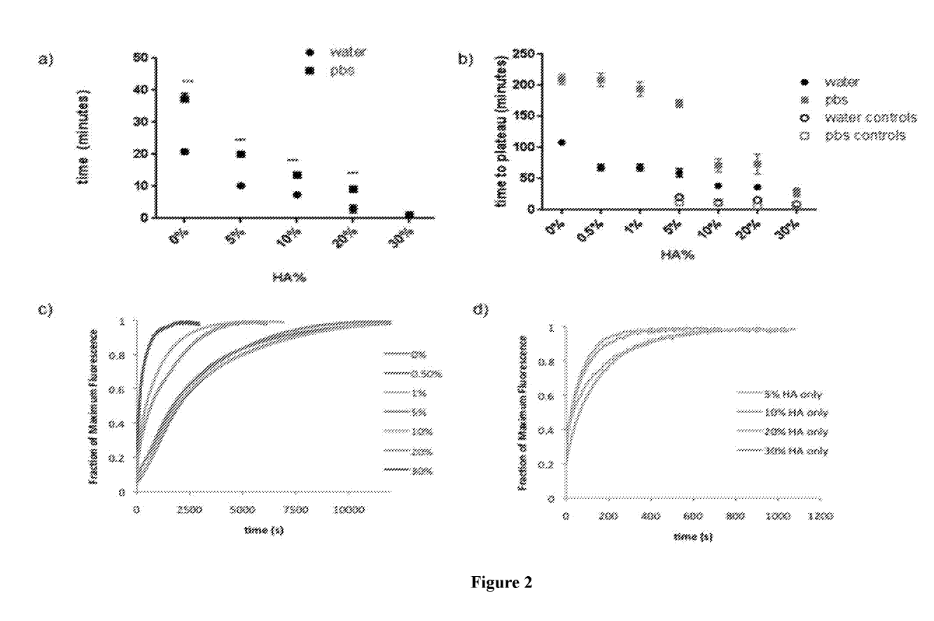

Similar trends in gelation time and crosslinking kinetics were seen with samples prepared with 0.5× PBS instead of water where higher concentrations of HA resulted in lower gelation times (FIG. 2). Samples made with PBS took much longer to gel than samples made with water. PBS control samples below that of 5% HA only did not form solid gels. As determined by fluorescence spectroscopy, PBS samples below that of 10% HA experienced a delayed response and the time to plateau decreased as HA content increased, similarly to samples prepared with water.

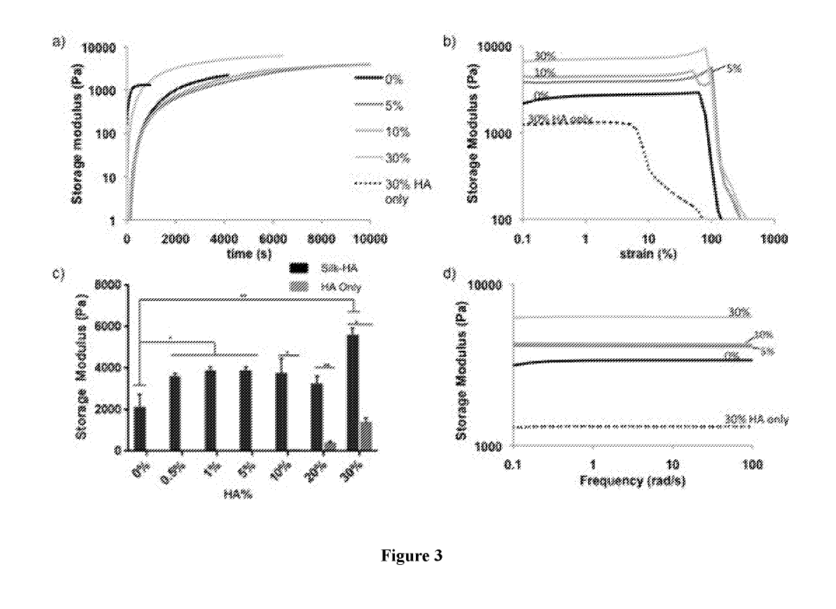

Gelation kinetics and shear mechanical properties were determined through rheology (FIG. 3). The time at which the hybrid hydrogels were mechanically stable were unaffected by HA content as seen by the time to plateau in FIG. 3, panel (a). However, similar to the fluorescence kinetics, HA only controls reached their maximum storage much faster than that of silk-HA hybrids. The silk-HA hydrogels exhibited higher elasticity, withstanding approximately 100% strain before failure. The HA only controls failed much sooner (˜10% strain) than that of the hybrid hydrogels. Hydrogels containing 0.5, 1, 5, and 30% HA exhibited a significantly higher final storage modulus than that of hydrogels with 0% HA. Additionally, hydrogels with only HA had a much lower final storage modulus as compared with hydrogels with silk and HA.

### Example 3

### Liquid Chromatograph and Mass Spectroscopy (LC-MS)

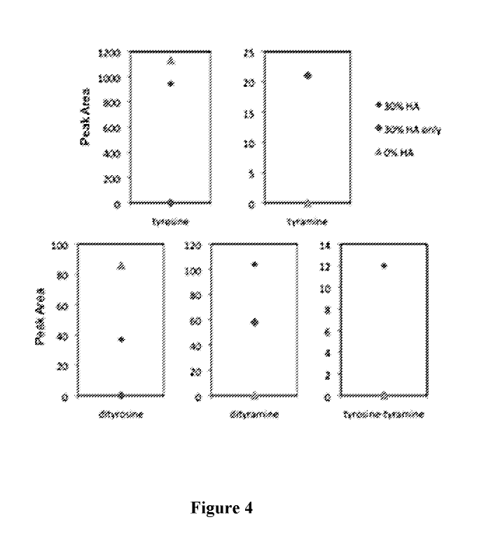

LC-MS was performed to determine the relative amount of the different types of crosslinks seen within the hydrogels. FIG. 4 shows the peak areas of each of the analytes for 0% HA, 30% HA, and 30% HA only. These results show that tyrosine and dityrosine analytes had the highest concentration in the 0% HA hydrogel followed by the 30% HA hydrogel. The 30% HA hydrogel had the highest concentrations of dityramine and tyramine-tyrosine. Finally, 30% HA only had the highest concentration of tyramine.

### Example 4

### Unconfined Compression

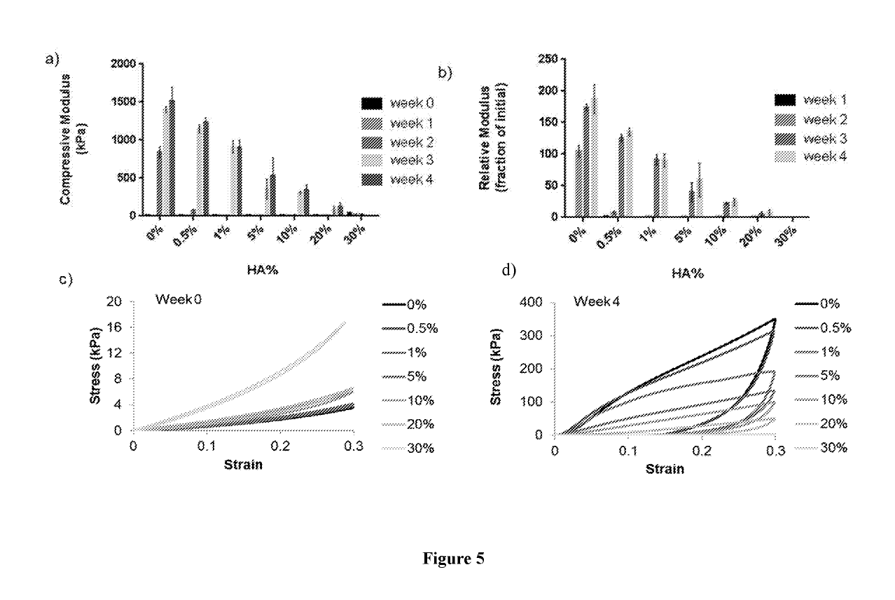

Dynamic mechanical analysis was performed to determine the unconfined compressive properties of the hydrogels over a period of 1 month (FIG. 5). After the hydrogels set over night and a subset of the samples was taken for the initial time point, then the rest of the gels were placed in 1× PBS at 37° C. and a subset was tested each a week for 1 month. During testing, a pre-load force of 0.5 g was placed to ensure full contact with the gel. The gels were then subjected to a single compression cycle to 30% strain. Initially, HA increased the stiffness of the hydrogels. The moduli of the 0% and 30% silk-HA samples, at day 0, were 8.06±0.77 and 40.16±7.23 kPa, respectively. The time at which the elastic moduli of the hydrogels increased was significantly delayed with the addition of higher HA content. For instance, hydrogels without HA experienced an increase in modulus at week 2 where, as hydrogels with 10% HA did not experience a significant increase until week 3. Additionally, there was a decrease in fold change over time with increasing HA concentration where at week 4, hydrogels with 0%, 1%, and 10% HA exhibited an estimated 200, 100, and 30-fold change in elastic modulus. A higher degree of resilience was also retained with higher HA concentrations, as seen in less hysteresis at week 4.

### Example 5

### Fourier Transform Infrared Spectroscopy (FTIR)

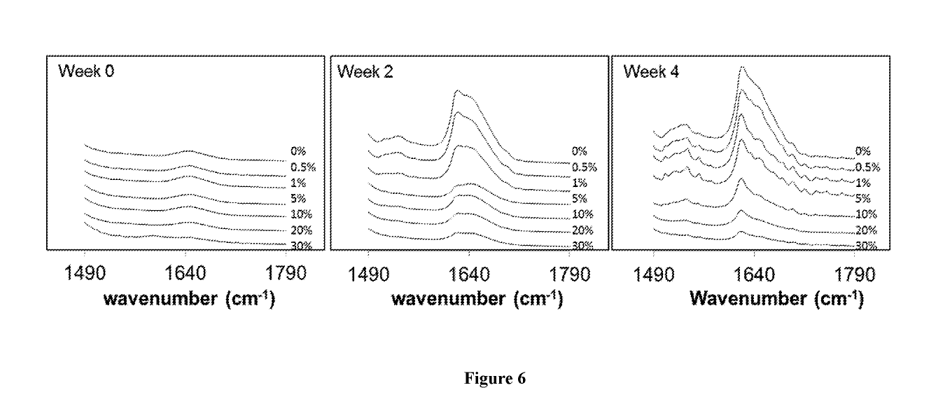

Secondary structure was assessed through FTIR to determine the conformational changes over time. Over 1 month, there was a peak shift in the FTIR spectra from ˜1640 cm⁻¹ to ˜1620 cm⁻¹ suggesting a change in silk secondary structure from random coil to crystalline β-sheet (FIG. 6). Initially at week 0, all samples had a small broad peak at around 1640 cm⁻¹. Increasing HA content tended to decrease the peak height and sharpness over time. This suggests that HA causes a stabilizing effect of silk's secondary structure over time. These differences in secondary structure are likely directly related to the mechanical stiffening of the hydrogels.

### Example 6

### Swelling and Opacity

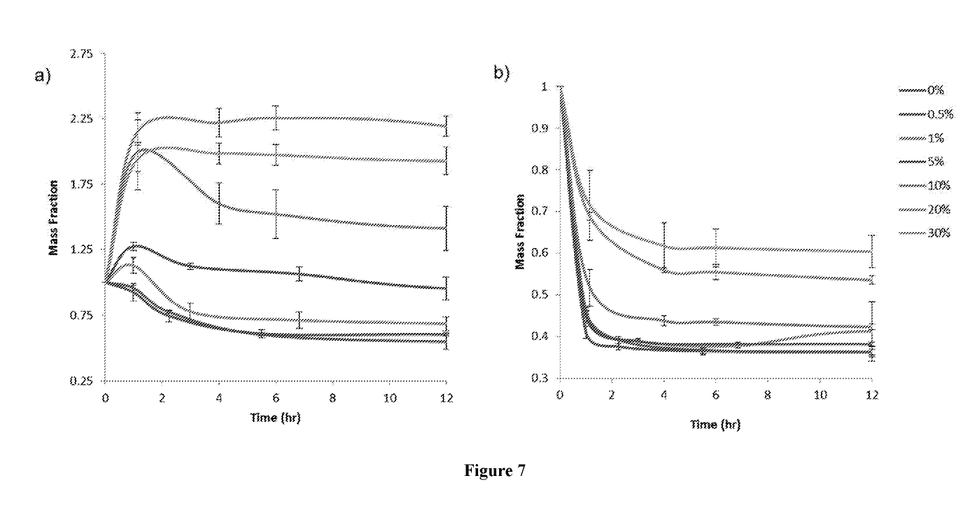

Swelling properties were evaluated by determining the mass of the hydrogels samples over time and normalizing to the initial mass before soaking in either ultrapure water or 1× PBS at 37° C. In ultrapure water, all samples above that of 0.5% HA experienced an initial increase in mass. The amount of increase was directly related to the amount of HA concentration within the 1% to 20% HA range. The 30% HA samples did not swell initially as much as that of the 20% HA samples. After the initial swelling, mass of the samples remained constant after 12 hrs. The fraction at which this occurred directly related to HA content, with increasing HA content increased final mass fraction. Visually, HA prevents diameter decrease over time as seen in FIG. 7.

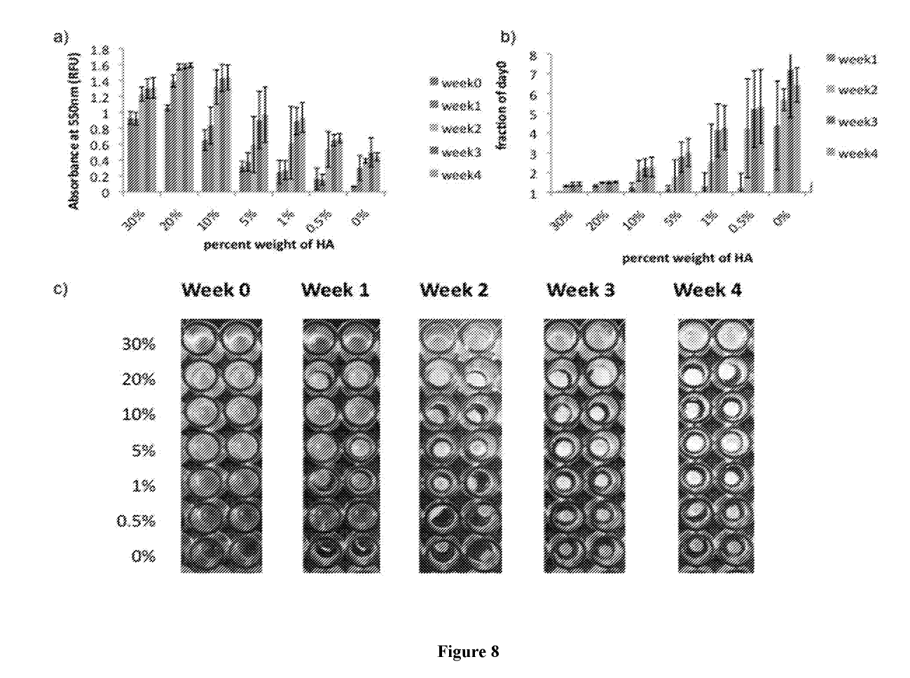

Due to visible changes in the hydrogels seen overtime, opacity was assessed by measuring the absorbance of the hydrogels at 550 nm. Initially, opacity increased with increasing HA content (FIG. 8, panels a and c). But overtime, increasing HA concentration decreased the fraction of initial absorbance closer to 1 (FIG. 8, panel b). This suggests that although HA content causes initial opacity, it prevents the increase in it over time.

### Example 7

### Optimization of Silk-(TS-HA) Hydrogels

During development of various embodiments, it was discovered that the formation of useful embodiments, could be, at least in part, highly dependent on the solubility and viscosity of the TS-HA. First, it was found that it was preferable for the TS-HA samples to be dissolved in DI water or PBS overnight prior to addition to silk fibroin. If added directly to silk, it was found that the silk will often aggregate and precipitate and the phenol-containing polymer (here HA) would not properly dissolve. The TS-HA solution was still very viscous after the initial dissolving (the highest possible concentration that can still be measured properly through a pipette was found to be ˜18mg/m1). The highest final HA concentration, after being added with silk, was found to be 8.6 mg/mL which resulted in a highly viscous, white solution but was still able to be accurately measured with a pipette. Higher HA concentrations resulted in phase separation and difficulties mixing and measuring.

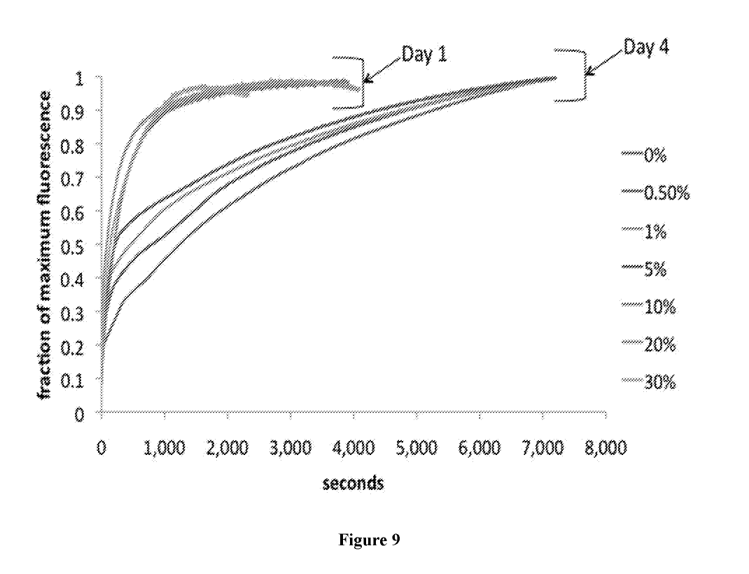

Additionally, it was determined that gelation using horseradish peroxidase (HRP) was highly dependent on that of pH. If pH was too high (pH ˜8), the solution did not gel, possibly due to the fact that this pH is out of range for ideal HRP activity (pH 6-6.5). Therefore pH variations in silk solution higher than that of 6.5 (which was uncommon) could impact results. Gelation kinetics was also dependent on the shelf life of the crosslinking components in solution. More specifically, hydrogen peroxide (diluted from a 30% v/v stock to a 1% v/v stock) was prepared and used initially and at day 4 for fluorescence kinetics. After 4 days, the fluorescence kinetics showed significantly different curves suggesting that the hydrogen peroxide may be unstable at 1% v/v (FIG. 9). From this, it was concluded that the 1% v/v hydrogen peroxide stock should be made fresh prior to each experiment.

Rheology was optimized to allow for testing of the samples, which gelled quickly by adding in the hydrogen peroxide while the sample was on the rheometer. The sample was then mixed using a 10 second pre-cycle at a steady shear rate as specified in the methods section.

The amount of polymer extracted from the hydrogels was determined after preformed hydrogels were soaked in sterile 1× PBS for 2 days on a shaker at room temperature. The silk fibroin recovered in solution was quantified using a Thermo Scientific™ Pierce™ BCA assay kit (Life Technologies, Carlsbad, Calif.).

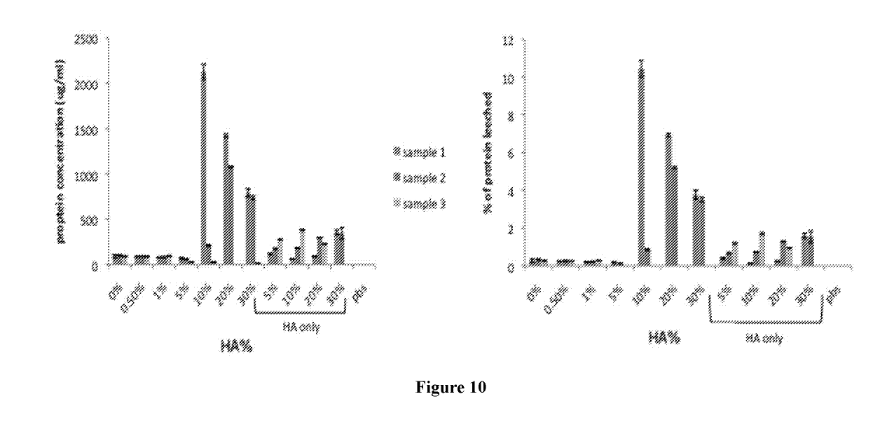

Initial data from BCA assays suggested that there are interfering substances where negative controls (HA only samples) showed a significant amount of protein levels (FIG. 10). Therefore prior to analyzing the protein concentration in the other Examples herein, protein was purified using trichloroacetic precipitation according to the manufacturer's protocol or a Coomassie Plus™ (Bradford) assay kit was utilized.

### Example 8

### Additional Exemplary Materials and Methods for Preparation of Silk-HA Hydrogels and Determination of Material Characteristics

Preparation of Aqueous Silk Solutions. Aqueous silk solutions were prepared using previously established methods. Briefly, sericin protein was removed from B. mori silkworm cocoons by placing 5 g of cut cocoons in 2 L of a boiling 0.02 M sodium carbonate solution (Sigma-Aldrich, St. Louis, Mo.) for 60 minutes. After rinsing in deionized (DI) water three times, the degummed fibers were dried overnight and solubilized in 9.3 M lithium bromide (Sigma-Aldrich, St. Louis, Mo.) for 4 hours at 60° C. The resulting silk solution was then dialyzed against DI water using standard grade regenerated cellulose dialysis tubing (3.5 kD MWCO, Spectrum Labs Inc, Rancho Dominguez, Calif.). After 6 changes over 3 days, insoluble silk particulates were removed by centrifugation (two times at 9000 RPM, 5° C., 20 minutes). Silk concentration was determined by weighing a dried sample of a known volume.

Lyophilized tyramine-substituted HA (Corgel® powder 2.8%, MW 0.9-1 MDa, Lifecore Biomedical, Chaska, Minn.) was dissolved in Ultrapure™ distilled water (ThermoFisher Scientific, Waltham, Mass.) overnight at 4° C. Aqueous silk and HA were combined through gentle pipetting to yield a final silk concentration of 20 mg/mL and final HA concentrations of 0, 0.2, 1.05, 2.22, and 5 mg/mL. Samples are referred to as the weight percentage of HA (Table 3).

TABLE 3
Sample Conditions. Samples are denoted by the mass percent
of tyramine-substituted HA as compared to the total polymer
concentration. Each condition was crosslinked with 10 U/mL
of HRP and 0.01% H₂O₂ (final molarity of 3.27 mM).

| | Silk Concentration | HA Concentration |
|---|---|---|
| Sample | (mg/mL) | (mg/mL) |
| 0% | 20 | 0.0 |
| 1% | 20 | 0.2 |
| 5% | 20 | 1.05 |
| 10% | 20 | 2.22 |
| 20% | 20 | 5.0 |
| HA only | 0 | 5.0 |

HA only hydrogels consisted of 5 mg/mL of HA. Crosslinking was initiated by adding of 10 U/mL of horseradish peroxidase (HRP, type VI, Sigma-Aldrich, St. Louis, Mo.) followed by 0.01% H₂O₂ (final molarity of 3.27 mM, Sigma-Aldrich, St. Louis, Mo. Composite hydrogels with HA concentrations above 5 mg/mL were excluded from experiments due to an increase in viscosity and phase separation resulting in difficulties with handing and reproducibility. After mixing, the solutions were allowed to gel for 3-4 hours at 37° C. prior to analysis, unless otherwise noted.

Gelation Kinetics. Gelation time was evaluated by a vial inversion test, where gelation time was designated as the time at which the solution no longer flowed after tilting the vials. Briefly, H₂O₂ was added into a silk-HA solution containing HRP in a 7 mL scintillation vial and mixed with a pipette for 10 seconds, yielding a total of 750 μL of hydrogel solution. Samples were kept at 37° C. where gelation was monitored (n=4). Crosslinking kinetics were assessed through fluorescence spectroscopy using a SpectraMax M2 multi-mode microplate reader (Molecular Devices, Sunnyvale, Calif.). A 200 μL aliquot of silk-HA solution was mixed in a black 96-well plate for 10 seconds with a pipette. Crosslinking was monitored at 37° C. by measuring the intensity of intrinsic fluorescence emission at 415 nm after excitation at 315 nm of the crosslinked phenolic groups until plateau. Results are reported as a fraction of the maximum intensity after subtracting a blank measurement taken prior to adding H₂O₂ (n=7).

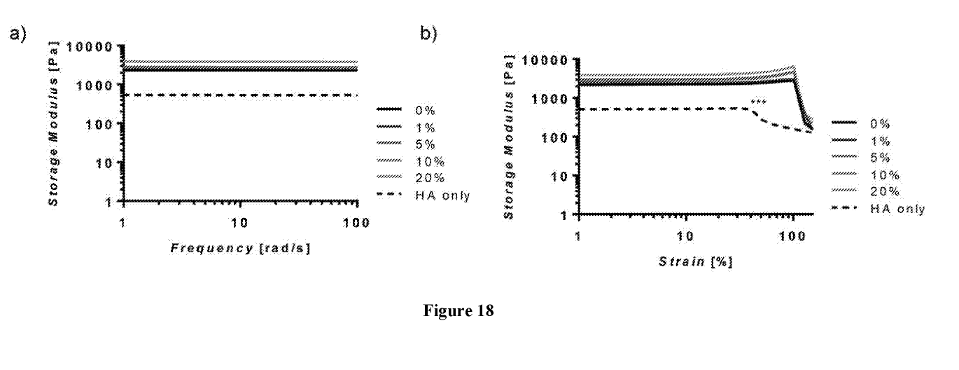

Determination of Rheological Properties. Rheological measurements were performed at 37° C. on a TA Instruments ARES-LS2 rheometer (TA Instruments, New Castle, Del.) using a 25 mm stainless steel upper cone and peltier bottom plate. Samples were mixed without the addition of H202. 415.8 μL of silk-HA solution with HRP was loaded onto the plate and the cone was lowered to 46.8 μm. During a 10 second precycle at a steady shear rate of 100/sec, 4.2 μL of 1% H2O2 was added into the gap. To prevent evaporation during analysis, mineral oil was placed around the system. A dynamic time sweep was performed at 1 Hz with a 1% applied strain until samples attained a steady modulus. Parameters were chosen in the linear viscoelastic region as determined in preliminary experiments (FIG. 18). Following gelation, dynamic frequency sweeps (0.1 to 100 rad/s at 1% strain) and strain sweeps (0.1% to 500% or to failure, whichever came first, at 1 Hz) were conducted (n=3).

Swelling and Wet Mass Determination. Percentages of water in the hydrogels were calculated using Equation 1.

$$Percent water = 100 \times \frac{( wet mass - dry mass )}{wet mass} . \qquad Equation 1$$

Briefly, wet mass was determined by blotting preformed hydrogels with a WypAll paper towel (Kimberly-Clark Co., Neenah, Wis.) to remove residual surface water and weighing. After drying in a 60° C. oven overnight, samples were weighed again to obtain their dry mass. The water content was determined initially and after incubation in 1× PBS at 37° C. for 1 day, and 1, 2, 3, and 4 weeks (n=5).

Fourier Transform Infrared Spectroscopy (FTIR). Secondary structure was analyzed by a JASCO FTIR 6200 spectrometer (JASCO, Tokyo, Japan) with a MIRacle™ attenuated total reflection with germanium crystal. Preformed hydrogels (4 mm diameter, ˜2-3 mm height) were washed in deuterated water (Sigma-Aldrich, St. Louis, Mo.) three times for 30 minutes each prior to analysis to remove the interference of water in the amide I region. Data was obtained by averaging 32 scans with a resolution of 4 cm-1 within the wavenumber range of 600 and 4000 cm-1. Samples were allowed to incubate in 1× PBS at 37° C. for 1 day prior to initial analysis and then at weeks 1, 2, 3, and 4 (n=5). Data was quantified by dividing the average peak absorbance at 1620-1625 and 1640-1650 cm-1 representing the ratio of β-sheet to random coil secondary structures.

In Vitro Degradation. Preformed hydrogels (˜22 mm diameter, 2 mm height) were first incubated at 37° C. in 1× PBS for 3-4 hours. The gels were then placed in 2 mL of an enzyme cocktail consisting of lyophilized hyaluronidase (type I-S from bovine testes, Sigma-Aldrich, St. Louis, Mo.), and protease (type XIV from Streptomyces griseus, Sigma-Aldrich, St. Louis, Mo.) dissolved at 1 U/mL and 0.001 U/mL in 1× PBS, respectively. The enzyme solution was changed every 2 days. At days 0, 1, 2, 4, 6, and 8 a subset of gels were removed from the enzyme solution and washed in Ultrapure™ water over night at room temperature, lyophilized and weighed. Results are shown as the mass fraction of the initial weight (n=4).

In Vitro Cell Response. Human mesenchymal stem cells (hMSCs) were isolated from human bone marrow aspirates (Lonza, Gaithersburg, Md.), as previously reported. Cells were cultured in growth media consisting of Dulbecco's Modified Eagle Medium (DMEM) supplemented with 10% fetal bovine serum, 1% Penicillin-Streptomycin (Life Technologies, Carlsbad, Calif.), and 1 ng/mL of basic fibroblast growth factor (bFGF) (Invitrogen, Carlsbad, Calif.). At passage 3, cells were plated onto preformed hydrogels (15 mm diameter, 1.5 mm height) containing silk only, silk-HA (10% HA), and HA only as well as tissue culture plastic (TCP) controls at a density of 10,000 cells/cm². Media was changed 3-4 hours after initial plating and then every 2-3 days. Brightfield images were collected initially after seeding and at day 7 with a BZ-X700 Fluorescence Microscope (Keyence Corp., Itasca, Ill.). Cell proliferation was quantified using Quant-iT PicoGreen dsDNA Assay Kit as per manufacturer's instructions (Invitrogen, Carlsbad, Calif.). In brief, cells were first lysed with 500 μL of 1× TE buffer solution (ThermoFisher Scientific, Waltham, Mass.). DNA content was determined in duplicates by measuring fluorescence intensity (ex/em: 480/520 nm) using a microplate reader after samples were diluted 5× with TE buffer and combined with PicoGreen reagent. Cell proliferation is shown as the fold change in DNA concentration at day 3, 5, and 7 compared to the initial DNA concentration (n=6).

Crosslinking Efficiency. The amount of polymer extracted from the hydrogels was determined after preformed hydrogels were soaked in sterile 1× PBS for 24 hours at room temperature. The supernatant was then removed and stored at −80° C. until analysis. The silk protein recovered in solution was quantified in triplicates using a Thermo Scientific™ Pierce™ Coomassie Plus™ (Bradford) assay kit (Life Technologies, Carlsbad, Calif.) according to the manufacturer's protocol. Silk solutions at known concentrations were used as standards. HA was quantified through a modified hexuronic acid assay performed in triplicates (see van den Hoogen et. al 1998, Anal. Biochem. 257 (2), 1998, 107-111). In brief, 200 μL of 120 mM of sodium tetraborate (Sigma-Aldrich, St. Louis, Mo.) in 96% w/w sulfuric acid (Fisher Scientific, Waltham, Mass.) was added to 40 μL of the sample in a clear 96-well plate and incubated for 1 hour at 80° C. After cooling to room temperature, background absorbance at 540 nm was collected using a SpectraMax M2 multi-mode microplate reader. Fifteen minutes after adding 40 μL of m-hydroxydiphenyl reagent (100 μL of m-hydroxydiphenyl in dimethyl sulfoxide, at 100 mg/mL, mixed with 4.9 mL of 80% v/v sulfuric acid), absorbance was measured again. Standards comprised of known HA concentrations between 50 and 300 mg/mL. Crosslinking efficiency was calculated by normalizing measured concentrations to theoretical amount of polymer that could be extracted.

Liquid Chromatography and Mass Spectroscopy (LC-MS). Covalent bonds were analyzed using an Agilent 1200 series LC in tandem with an Agilent 6410 triple-quadruple MS/MS (Agilent Technologies, Santa Clara, Calif.) operated in positive electrospray ionization mode. Preformed gels were hydrolyzed in 6N HCl (Sigma-Aldrich, Saint Louis, MO) with 1% w/v phenol (Invitrogen, Carlsbad, Calif.) for 4 hours at 120° C. Hydrolyzed samples were dehydrated and then reconstituted in 1 mL of 75% v/v LCMS grade acetonitrile in water (Fisher Scientific, Waltham, Mass.) and pipette mixed to homogenize. Samples were diluted 200× for analysis and twenty microliter samples were injected into a hydrophobic interaction liquid chromatography column (Zorbax HILIC Plus, 4.6 mm×100 mm, 3.5 μm, Agilent Technologies, Santa Clara, Calif.) at 40° C. and a rate of 1.0 μL/min. Samples were first equilibrated at 95:5 acetonitrile (0.1% v/v formic acid) to water (0.1% v/v formic acid). After 1 minute the mobile phase was adjusted to 5:95 acetonitrile to water over 5 minutes, held at high water for 2 minutes, and then returned to high acetonitrile. Nitrogen gas was operated at a flow rate of 11 L/min and set to 300° C. Fragment ions for each analyte of interest were identified using a product scan, and the collision energy for each ion was optimized to yield the maximum product. This information was integrated into a multiple reaction monitoring (MRM) program in the Agilent Mass Hunter software. Crosslinks were identified in samples with peak areas above that of negative controls.

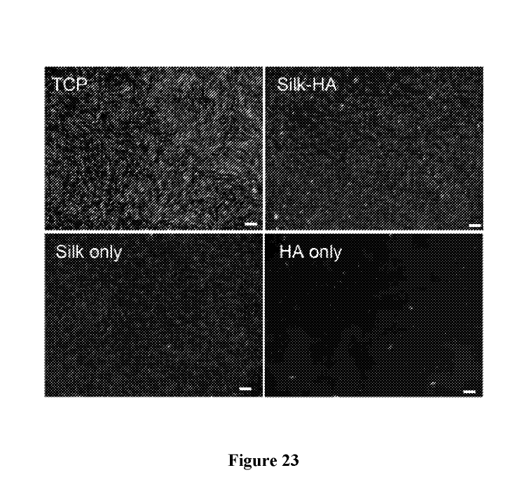

Cell Viability. The viability of hMSCs were analyzed using LIVE/DEAD® Cell Imaging Kit (Life Technologies, catalog number: R37601, Carlsbad, Calif.), after being cultured for 3 days on TCP, silk only, silk-HA (10% HA), and HA only hydrogels (see FIG. 23). Samples were imaged using a BZ-X700 Fluorescence Microscope.

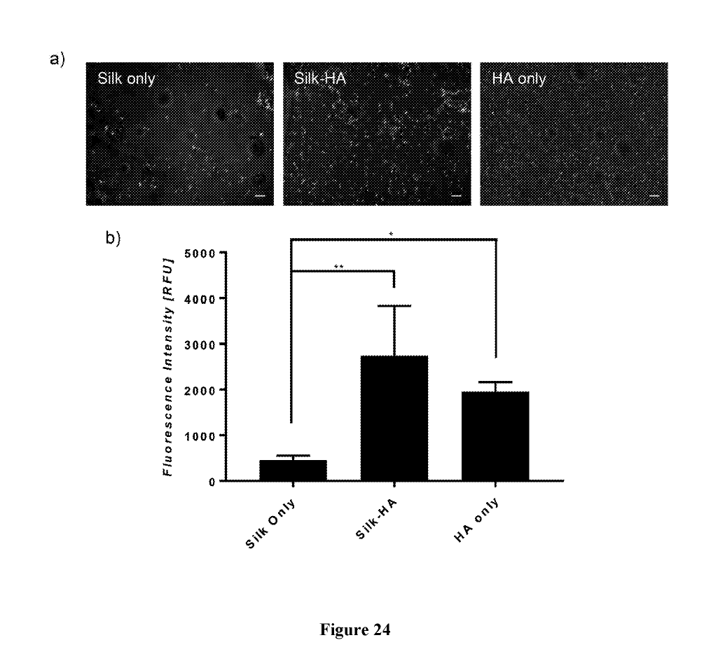

Cell Encapsulation. Silk only, silk-HA (10% HA), and HA only hydrogels for cell encapsulation were prepared to contain a final 0.5× concentration of growth media (DMEM supplemented with 10% fetal bovine serum, 1 ng/mL of basic fibroblast growth factor, and 1% Penicillin-Streptomycin). At passage 4, hMSCs, which were isolated and cultured as described in the In Vitro Cell Response section of the Materials and Methods,were suspended in the hydrogel, 5-10 minutes prior to gelation, at a density of 1*106 cells/mL, and 100 μL of hydrogel solution containing cells was plated into sterile 8 mm diameter polydimethylsiloxane (PDMS) molds. After 10 minutes at 37° C., to allow for adequate gelation, the samples were flooded with growth media. After 2 hours, the samples were removed from the molds, and placed into fresh media. At day 3, the viability of hMSCs was assessed using LIVE/DEAD® Cell Imaging Kit and imaged using a BZ-X700 Fluorescence Microscope (n=3; see FIG. 24). Metabolic activity at day 3 was determined through AlamarBlue (Life Technologies, Carlsbad, Calif.) as per manufacturer's instructions (see FIG. 24). Briefly, AlamarBlue reagent diluted 1× with cell culture media was added to the samples and incubated for 4 hours at 37° C. Then 200 μL of the resulting media was placed in a black 96-well plate and fluorescence intensity at 560/590 nm was measured with a SpectraMax M2 multi-mode microplate reader (n=4).

Statistical Analysis. Data are expressed as means±standard deviations. One- or Two-way ANOVA (analysis of variance) with Tukey's post hoc multiple comparison tests were performed using GraphPad Prism (GraphPad Software, La Jolla, Calif.) to determine statistical significance (*p≤0.05, **p≤0.01, ***p≤0.001).

### Example 9

### Gelation of Silk-HA Hydrogels

The formation of composite silk hydrogels with controllable material properties offer many clinically relevant advantages. The enzymatic crosslinking method, using HRP and H₂O₂, has shown minimal cytotoxicity with the in situ gelation of HA hydrogels. In order to establish this method of use, the effects of incorporating a hydrophilic bioactive polymer were tested in order to characterize gelation properties over time.

As described herein, in some cases, HA concentration altered gelation kinetics, where increasing HA concentration above 1% steadily decreased the sol-gel transition time from 20 minutes to just over 1 minute. A similar trend was seen with crosslinking kinetics where HA concentration was inversely correlated to the completion of crosslinking. Without wishing to be held to a particular theory, these results may be in part due to an increase in potential crosslinks from an increase in HA concentration. The ability to tune the sol-gel transition within this time frame shows the potential for in situ gelation. Since there are no known human peroxidases that facilitate phenolic crosslinking, in order to let HRP crosslinked hydrogels form in situ, sufficient time prior to gelation after mixing is required.

Without wishing to be held to a particular theory, it may be possible to achieve similar results by crosslinking silk to other high molecular weight, unbranched, hydrophilic polymers such as polyethylene glycol, extending the utility of the system. Additionally, other silk-based hydrogels using a similar method of preparation, showed mechanical properties and extent of stiffening controllable by altering silk molecular weight and concentration, as well as the ratios of the cross-linking components, which if applied to the silk-HA hybrid system, can further the versatility and tunability of the hydrogels.

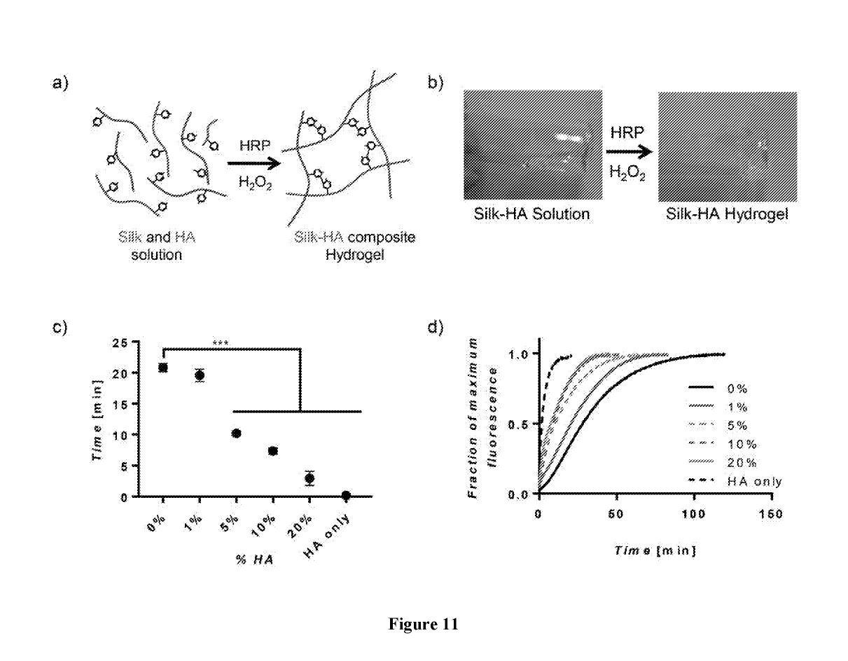

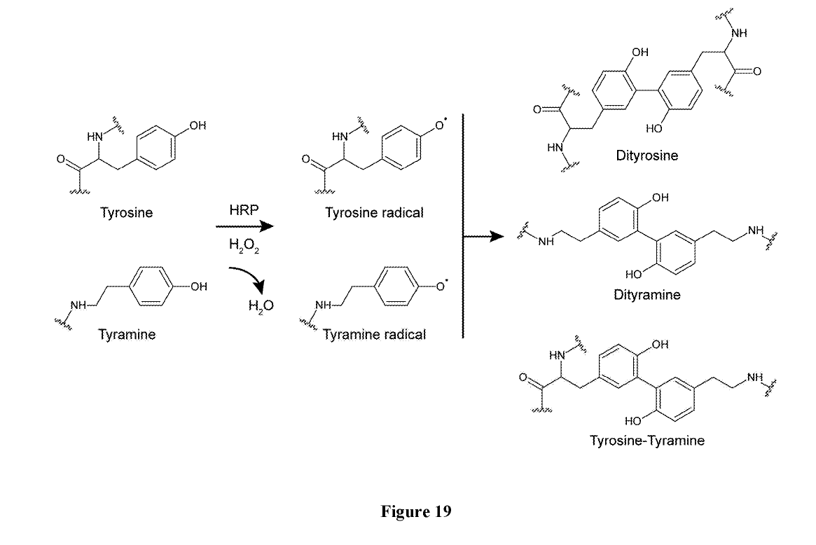

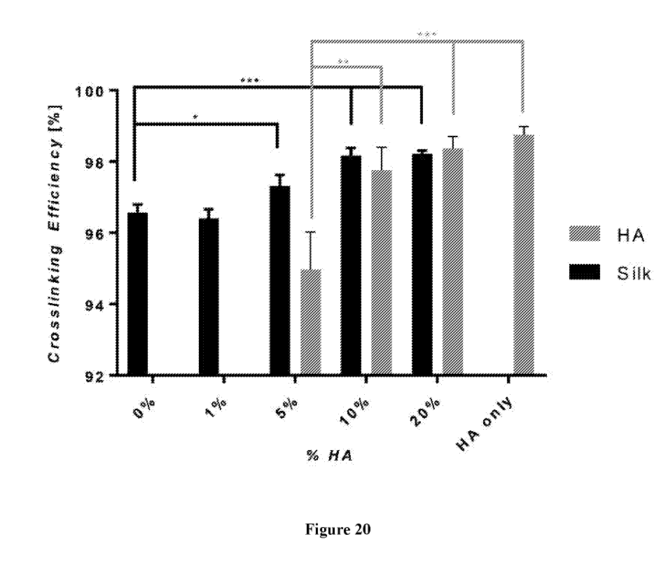

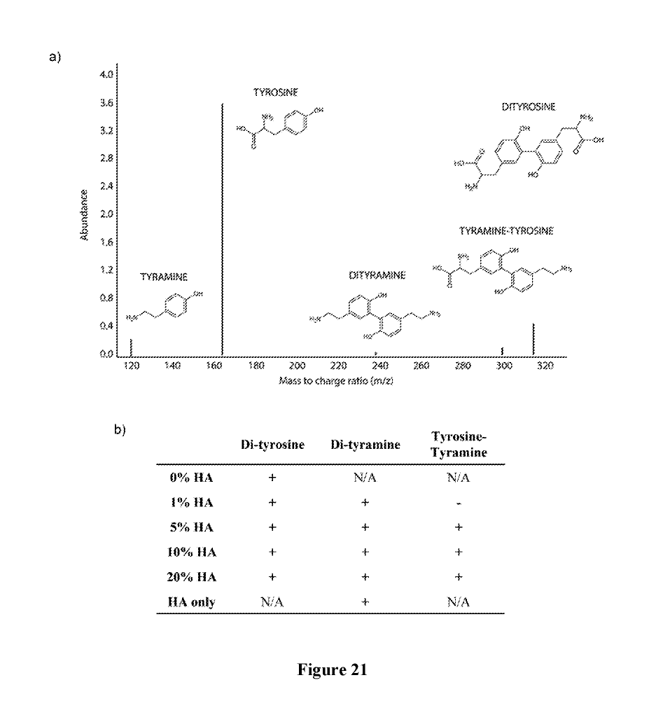

Polymerization of the precursor hydrogel solutions with HRP and H₂O₂ generated phenolic radicals that could covalently bond with one another (FIG. 19). In the silk-HA system, this reaction covalently linked the two polymers (FIG. 11, panel a), forming a hybrid, composite hydrogel with polymer crosslinking efficiency above 94% (FIG. 20). The types of crosslinks that are possible within the composite system include dityrosine (silk-silk bonds), dityramine (HA-HA bonds), and tyramine-tyrosine (silk-HA bonds) (FIG. 21, panel a). All three crosslinks were identified in all composite hydrogels except that of 1% HA, indicating that the hydrogels contained both inter and intra-polymer bonds (FIG. 21, panel b). In the 1% HA samples, where the concentration of tyramine was relatively low initially, dityramine and tyramine-tyrosine peaks had similar areas to that of negative controls, and therefore they could not be reliably analyzed.

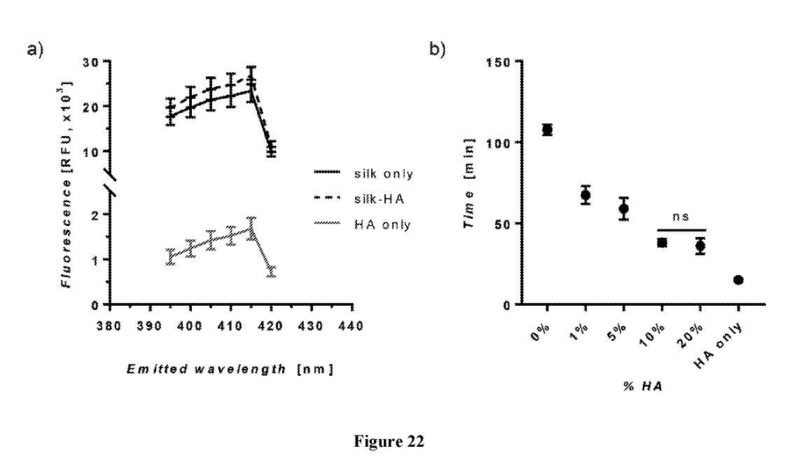

The sol-gel transition of silk-HA hybrid hydrogels ranged from a few to approximately 20 minutes, as determined by a vial inversion test. When increasing the HA concentration greater than 1% gelation time decreased significantly, compared to silk only hydrogels (FIG. 11, panel c). As expected, HA only hydrogels gelled within seconds after crosslinking was induced. To further assess gelation properties, crosslinking kinetics were explored by quantifying the rate of formation of phenolic crosslinking, which intrinsically emit fluorescence at 415 nm after excitation at 315 nm (FIG. 22, panel a). Both the shape of the curve and the time to plateau were dependent on HA content (FIG. 11, panel d). Silk only hydrogels experienced a delay in crosslinking, showing a sigmoidal shaped curve, whereas composite and HA only hydrogels showed hyperbolic shaped curves with no delay. HA only and silk only hydrogels completed crosslinking in approximately 15 and 110 minutes, respectively.

Composite hydrogels completed crosslinking between these two extremes in which increasing HA concentration, lead to decreased crosslinking times (FIG. 22, panel b).

### Example 10

Rheological Properties of Silk-HA Hydrogels

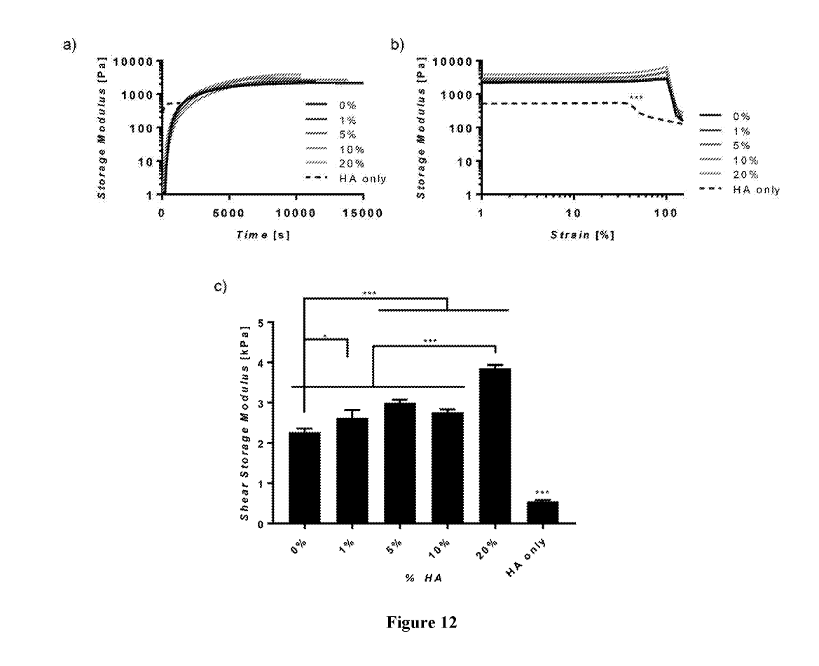

Shear mechanical properties and the dependence on polymer concentration were determined through rheology. As described, the time to reach stable shear mechanics can be decreased by increasing silk concentration and decreasing molecular weight, thus potentially increasing the rate of intramolecular silk interactions. Herein, since silk concentration and molecular weight were held constant, HA concentration had no effect on the time to reach stable shear mechanics, and the silk component in the composite system drove initial mechanical stability. However, the addition of HA, led to increased moduli as compared to silk only hydrogels, due to the increase in overall polymer concentration and potential crosslinking sites. For pure silk and silk-HA hydrogels, mechanical stability occurred between 132 and 174 minutes, with no significant dependence on HA concentration (FIG. 12, panel a). However, HA only hydrogels reached their maximum storage modulus at ˜15 minutes, much faster than that of silk only and silk-HA hybrids, revealing that silk component in the hybrid hydrogels is the limiting factor in mechanical stability during gelation. The silk and silk-HA hydrogels withstood approximately 100% strain before failing, whereas the pure HA hydrogels starting fracturing at ˜30% strain (FIG. 12, panel b). Pure silk and pure HA hydrogels had storage moduli of 2.27 ±0.09 and 0.55 ±0.03 kPa, respectively. All composite hydrogels exhibited higher final storage moduli than that of the pure hydrogels, but those containing 20% HA showed the highest at 3.85 ±0.08 kPa (FIG. 12, panel c).

### Example 11

### Unconfined Compression

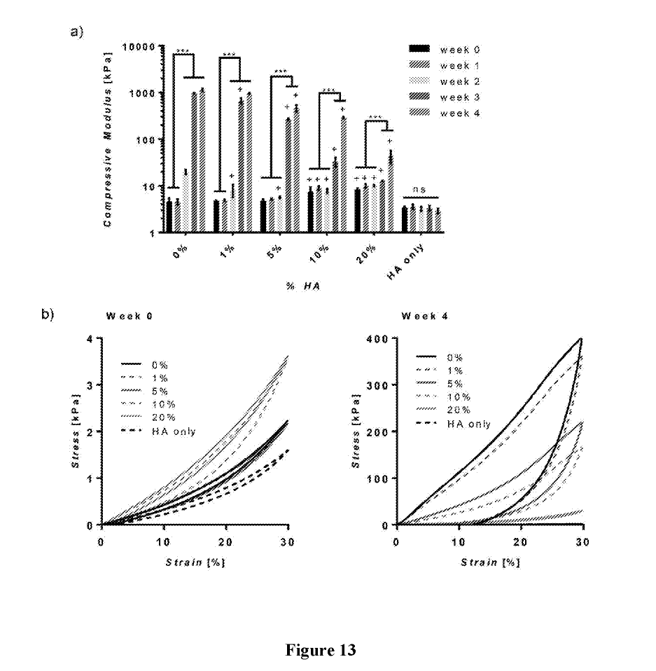

A notable effect of HA concentration in the composite hydrogels was the control of mechanical changes over time. The formation of composite hydrogels results in the preservation of mechanical properties of including mechanics, elasticity, and water retention over time and offers a favorable material over silk or HA alone for in vivo tissue engineering applications. It was found that the high HA concentration impedes the extensive changes in mechanical properties. Mechanical analysis was executed to demonstrate the unconfined compressive properties of the hydrogels over a period of 1 month (FIG. 13). Preformed hydrogels were soaked in PBS at 37° C. and a subset of the samples were analyzed for compressive properties at weeks 0, 1, 2, 3, and 4. Initially, all hydrogels had moduli ranging between ˜3 and 8 kPa and fully recovered as seen in the hysteresis curves (FIG. 13). The times at which the elastic moduli of the hydrogels increased were significantly delayed with the addition of higher HA content. For instance, hydrogels containing 0 and 1% HA experienced a small increase in modulus at week 2 with a much larger increase at week 3. On the other hand, hydrogels with 5, 10, and 20% HA did not begin to stiffen until week 3 and HA only hydrogels remained consistent at ˜3 kPa (FIG. 13, panel a). At week 4, all hybrid hydrogels except that of 1% HA were less stiff than silk only hydrogels. Particularly, increasing the amount of HA reduced the magnitude of stiffening. For instance, at week 4, hydrogels with 0, 1, 5, 10, and 20% HA stiffened approximately 240, 200, 100, 40, and 5 times their initial modulus (Table 4).

TABLE 4
Fractions of initial modulus were calculated to show the change
in moduli over time and the dependency on HA concentration.

| | Week 1 | Week 2 | Week 3 | Week 4 |
|---|---|---|---|---|
| 0% | 0.99 ± 0.22 | 4.30 ± 0.94 | 207.42 ± 38.93*** | 242.95 ± 48.68*** |
| 1% | 1.03 ± 0.05 | 1.71 ± 0.52 | 140.19 ± 20.22*** | 201.07 ± 8.12*** |
| 5% | 1.10 ± 0.11 | 1.20 ± 0.13 | 56.22 ± 6.34*** | 97.76 ± 19.46*** |
| 10% | 1.19 ± 0.32 | 1.04 ± 0.28 | 4.44 ± 1.48 | 38.54 ± 9.59*** |
| 20% | 1.19 ± 0.13 | 1.20 ± 0.08 | 1.53 ± 0.10 | 5.19 ± 1.65 |
| HA only | 1.05 ± 0.14 | 0.95 ± 0.12 | 0.97 ± 0.14 | 0.85 ± 0.12 |

(n = 5, ***p ≤ 0.0.001 compared to week 7).

A higher degree of resilience was also retained with higher HA concentrations, as seen through less hysteresis during a single load-unload cycle at week 4 (FIG. 13, panel b). Additionally, hydrogels containing 10% HA or less did not fully recover (FIG. 13, panel b).

### Example 12

### Assessment of Composite Hydrogel Secondary Structure: Fourier Transform Infrared Spectroscopy (FTIR)

In additional to mechanical properties of composite silk-HA hydrogels, secondary structure was assessed through FTIR to determine the conformational changes over time.

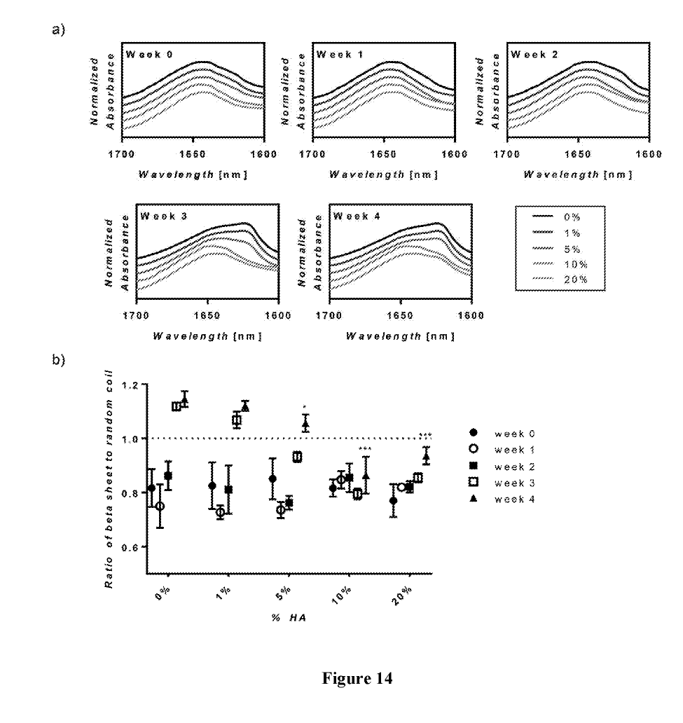

Over 1 month, there was a peak shift in the FTIR spectra from ˜1640 cm⁻¹ to ˜1620 cm⁻¹ in silk only and low HA concentration hydrogels, suggesting a change in silk secondary structure from random coil to crystalline β-sheet (FIG. 14, panel a). This shift was quantified by calculating the ratio of the average peak absorbance at 1620-1625 and 1640-1650 cm⁻¹ (FIG. 14, panel b). At week 3, the ratio of 0% and 1% HA hydrogels increased to over 1, signifying there was a larger contribution from β-sheet than random coil secondary structures. The 5% hydrogels followed this trend at week 4. Additionally, at week 4, the 5%, 10% and 20% hydrogels had significantly lower ratios as compared to silk only hydrogels.

### Example 13

### Water Retention of Composite Silk-HA Hydrogels

The HA contains favorable properties for long-term in vivo applications and contributes to the characteristics of the composite hydrogel. One of these properties is enhanced hydrogel water retention.

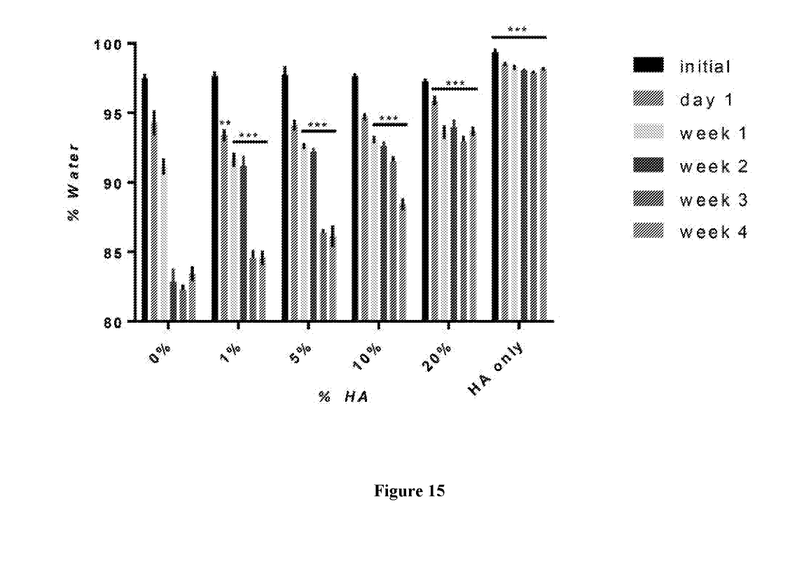

To demonstrate the effects of these characteristics in composite hydrogel form, he water retention capabilities of the hydrogels were determined by calculating percentage of water in the hydrogels over 1 month (Equation 1). FIG. 15 shows that initially, all composite hydrogels contained ˜97% water, similar to that of silk only hydrogels. Starting at week 1, the composite hydrogels contained higher percentages of water as compared to silk only hydrogels. HA concentration was directly correlated to water retention, where after 1 month the 0, 10, and 20% HA hydrogels contained 83%, 88%, and 94% percent water, respectively. This shows that increased HA concentrations help maintain water content over time.

### Example 14

### In Vitro Degradation of Composite Silk-HA Hydrogels

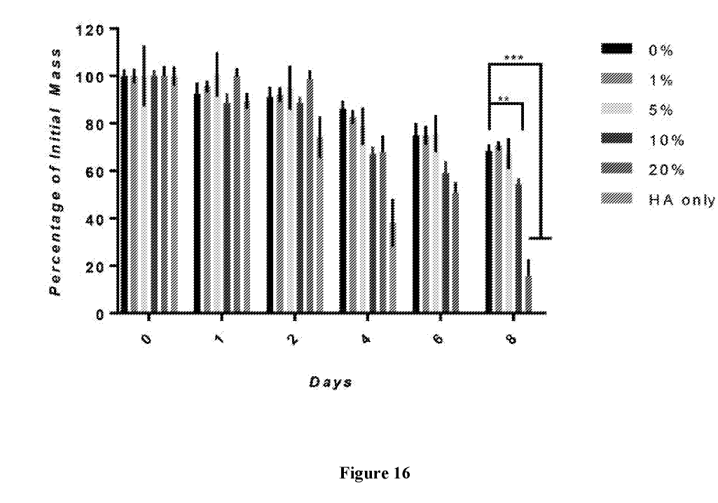

Advantages of a composite hydrogel are also demonstrated through the ability of the silk to contribute to the increased resistance to degradation over time of the composite material. In order to characterize these effects, the degradation profiles of hydrogels were determined in vitro after soaking hydrogels in an enzyme solution consisting of protease and hyaluronidase (FIG. 16). The composite hydrogels exhibited degradation rates ranging between that of their singular component counterparts. HA only hydrogels completely degraded at day 6 whereas silk only hydrogels were 70% their original weight at day 8. In the composite hydrogels, HA concentration was correlated to degradation rate as increasing HA concentration above 5% increased degradation at day 8. More specifically, hydrogels with 10 and 20% HA were approximately 50% and 15% their initial weight, respectively.

### Example 15:

### hMSC Responses

Previous methods of crosslinking hydrogels have been shown to be carried out under physiological conditions, however, the hydrogels are often washed prior to injection to extract the not reacted crosslinking agents which are cytotoxic and can lead to loss of cell and tissue functions. The present method of enzymatically crosslinking silk and HA can provide an alternative, cytocompatible method of forming composite hydrogels.

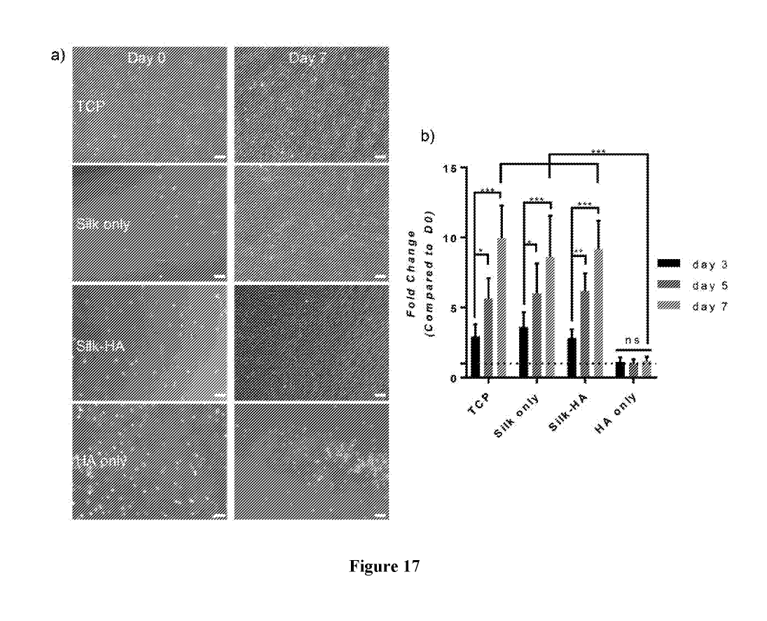

To demonstration cytocompatibility of the composite hydrogrels, hMSCs from bone marrow aspirates were plated onto preformed hydrogels containing silk only, silk-HA, HA only, or TCP to determine in vitro cell response. Brightfield images showed cell attachment and morphology at day 0 and 7, where cells on TCP, silk, and silk-HA (10% HA) hydrogels became confluent (FIG. 17, panel a). Cellular proliferation at day 3, 5, and 7 are shown as the fold change of initial DNA content, quantified with PicoGreen (FIG. 17, panel b). Both silk and silk-HA hydrogels supported cellular growth with spread morphologies and proliferation, similar to that of TCP controls, revealing the cytocompatibility of the hydrogels. On the other hand, HA only hydrogels inhibited hMSC growth with cell morphology remaining spherical over 1 week.

### Example 16

### SEM Images of Hydrogels Before and After In Vitro Degradation

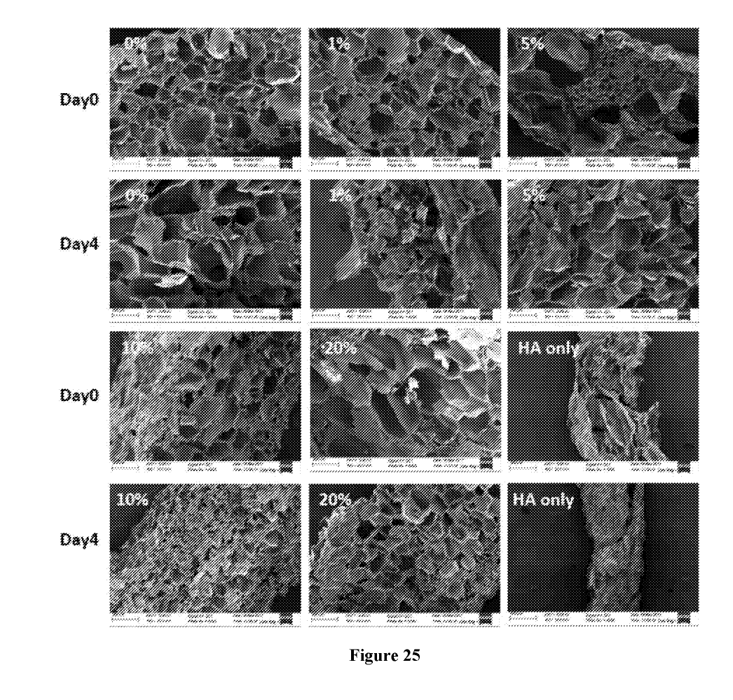

As is shown in FIG. 25, scanning electron microscopy (SEM) imaging was performed on hydrogels initially and after 4 days of incubation in 1 U/mL of hyaluronidase (type I-S from bovine testes) and 0.001 U/mL of protease (type XIV from Streptomyces griseus). Prior to imaging, hydrogels were rinsed in water, frozen, lyophilized, and gold sputter coated. Sample information is found below in Table 5:

TABLE 5
Sample Information

| | Silk | TS-HA* |
|---|---|---|
| Sample | (mg/mL) | (mg/mL) |
| 0% | 20 | 0.0 |
| 1% | 20 | 0.2 |
| 5% | 20 | 1.05 |
| 10% | 20 | 2.22 |
| 20% | 20 | 5.0 |
| HA only | 0 | 5.0 |

*TS-HA = tyramine substituted hyaluronic acid

SEM images were taken in hopes to determine morphology and pore size of the hydrogels, but this could not be accomplished due to alterations in the hydrogel properties during sample preparation. Specifically, due to the high water content of the materials, the freeze-drying process resulted in scaffold collapse and artifacts from ice crystal formation. Therefore the SEM images do no accurately represent the native hydrogel morphology and further conclusions cannot be made.

Preliminary Encapsulation of hMSCs without DMEM

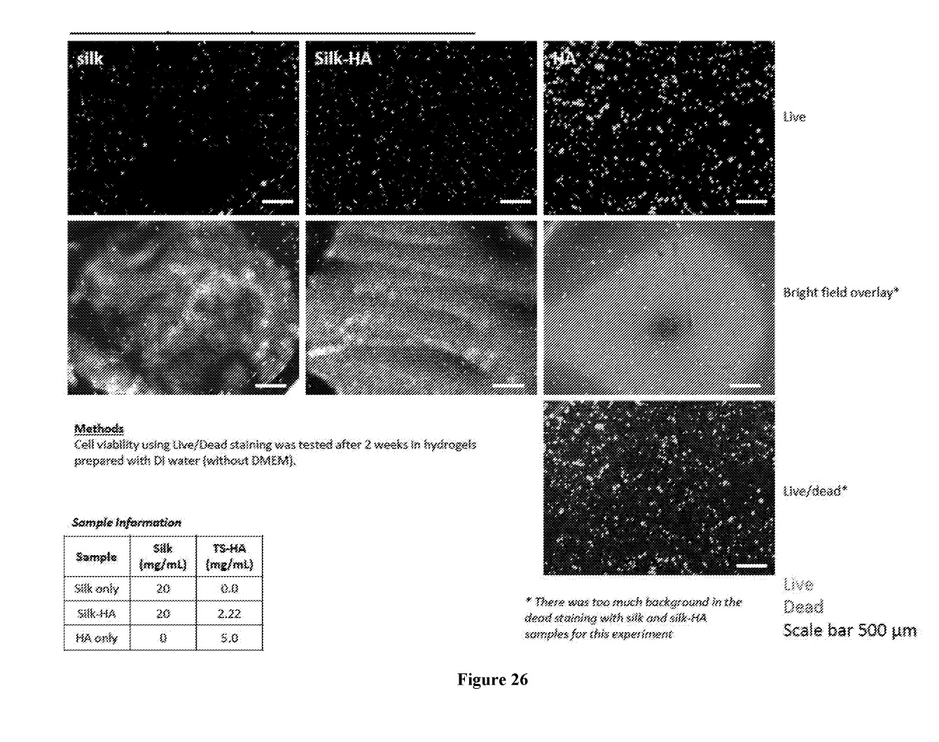

Cell viability using LIVE/DEAD® Cell Imaging kit was visualized using a florescent microscope 2 weeks after hMSCs were encapsulated within hydrogels prepared with DI water (without DMEM). For specific methods on cell culture and encapsulation see the “Cell Encapsulation” section of Example 8 (where everything is similar except the hydrogels were made with water instead of a final 0.5× concentration of DMEM). Since the hydrogels were made with water, we expected minimal cell viability due to cell lysis upon differences in osmotic pressure. But we observed that some cells remained viable in the hydrogel, showing the potential as a material to encapsulate cells (see FIG. 26).

### Example 17

### CD44 Staining of hMSCs

At day 3 after seeding, both 2D surface seeding and 3D encapsulation samples were stained with anti-CD44 (green) and Dapi (blue). At day 3, samples were washed in 1× PBS and fixed in 10% phosphate buffered formalin for 10-12 minutes for 2D surface seeding and 2 hours for 3D encapsulated cells. Samples were then washed in 1× PBS. Samples were permeabilized in 0.1% Triton X-100 for 5 and 20 minutes for 2D and 3D samples respectively. Then samples were washed in a blocking solution containing 1% bovine serum albumin in PBS with 0.1% Tween-20 for 5 and 20 minutes for 2D and 3D samples respectively. Samples were incubated with anti-CD44 (conjugated to AlexaFluor 488, 1:50) overnight, washed in 0.1% PBS-Tween. Samples were then stained with phalloidin and Dapi prior to imaging with a fluorescent microscope.

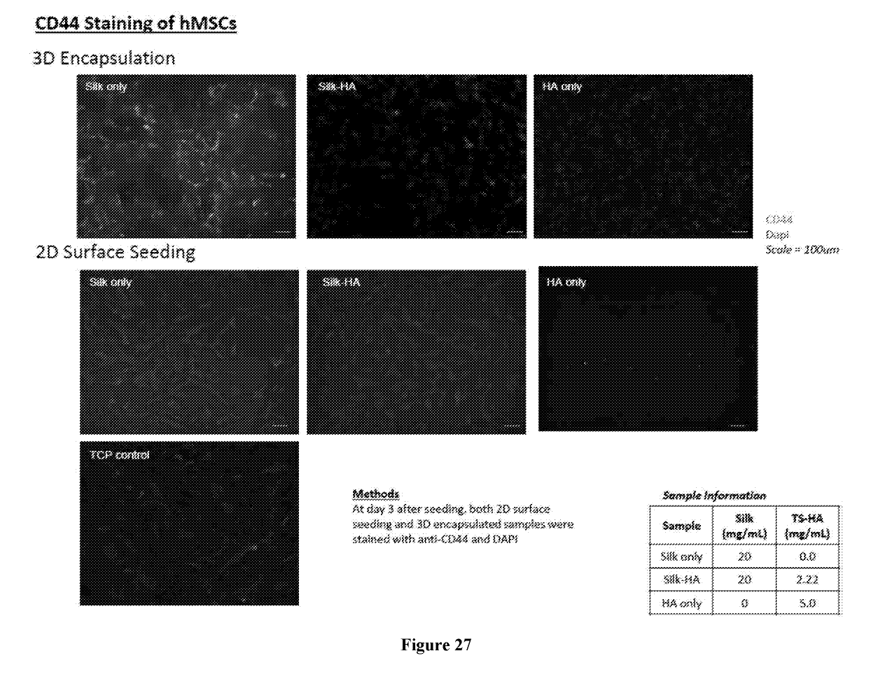

Cells are known to interact with hyaluronic acid through the cell receptor, CD44. But due to the limited expression of CD44 in the images obtained and the lack of adhesion to the HA only hydrogel control, the results were not clear (see FIG. 27).

### Example 18

### DAPI/Phalloidin Staining of 2D hMSCs on Hydrogels

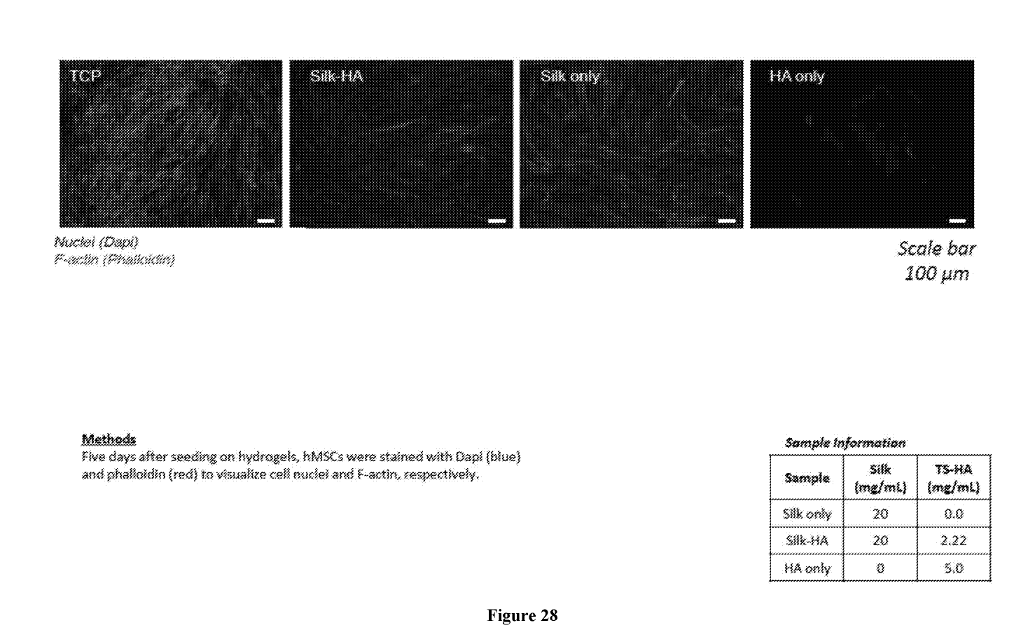

Five days after seeding onto hydrogels, hMSCs were stained with Dapi (blue) and phalloidin (red) to visualize cell nuclei and F-actin, respectively. Samples were prepared as described above. After incubation with anti-CD44, samples were washed with PBS-tween (0.1%) 3 times and rinsed with blocking solution. Samples were then incubated with phalloidin (1:100) and Dapi (1:1000) for 1 hour, washed in PBS, and imaged using a fluorescent microscope (see FIG. 28).

Cell morphology was similar on silk-HA and silk only hydrogels as compared to TCP controls showing that they hydrogels are cytocompatible and support cell attachment. There were minimal cells on HA only hydrogels, showing that they do not support cell attachment.

### Example 19

### Injection Force Testing

Hydrogels (1 mL) were allowed to gel in a 1 mL syringe for >3 hours. After gelation, the force required to inject the hydrogels through a 21G thin-wall 1 inch needle at 1 mm/s for 25 seconds were recorded with an Instron attached to a 100 N load cell.

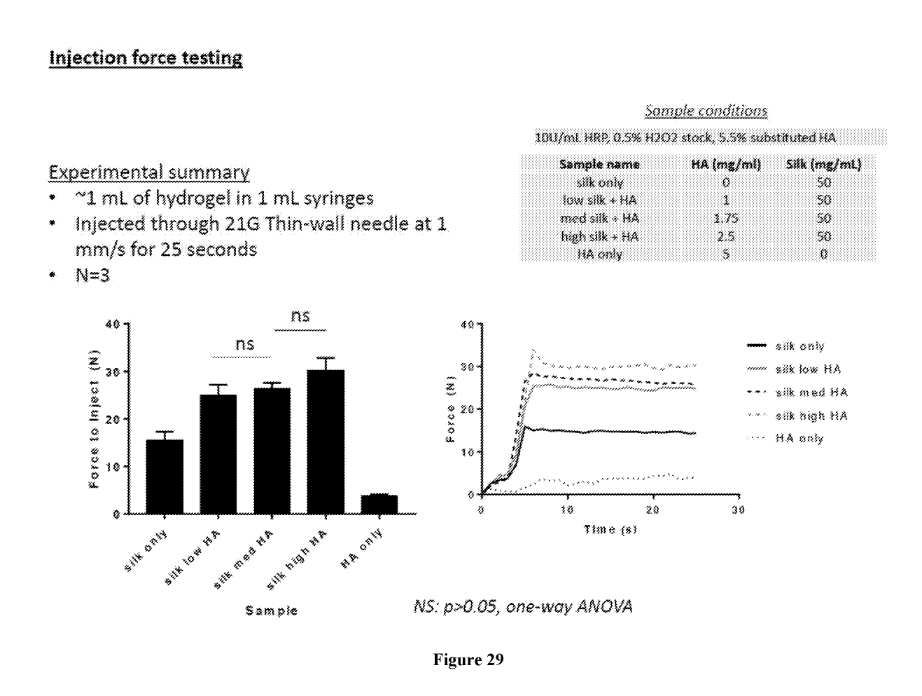

All hydrogels had constant injection force profiles with forces under 40 N, which shows that the hydrogels can be easily injected (see FIG. 29).

### Example 20

### Mechanical Properties of Cervical Tissue Prior to and after Injection of Hydrogels

Cervical tissue was obtained from nonpregnant women undergoing hysterectomy for benign indications (IRB #8315). Samples (10 mm diameter×8 mm height) were treated with 2 mg/mL collagenase (˜0.4 U/mL) for 2 hrs at 37 C. Unconfined compression of the treated samples were performed initially (pre-injection) and then after injecting 300 μL of a silk-HA hydrogel (post-injection) using a RSA3 dynamic mechanical analyzer. In brief, load-unload cycles at a strain rate of 1 mm/min up to 20% strain were performed. The cycle was repeated 3 times and on the last cycle, the modulus was calculated between 1 and 5%.

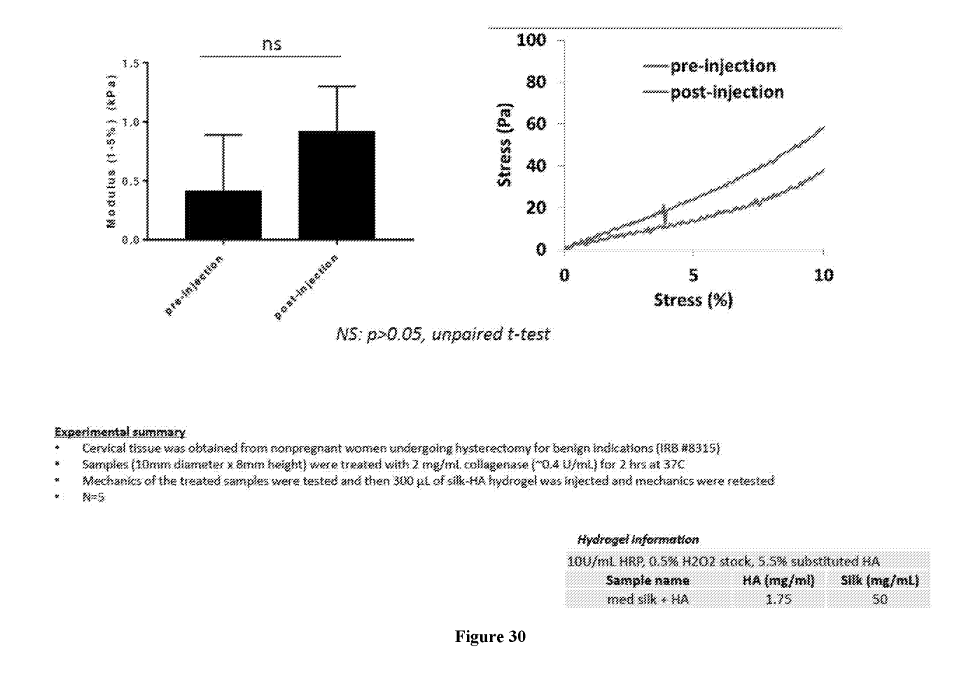

In the context of this project, we are aiming to bulk cervical tissue without significantly altering mechanics in hopes to provide an alternative approach to prevent preterm birth. In this experiment, we determined that there was a slight but not significant increase in modulus after injection, therefore showing the potential of the injectable hydrogel as a bulking material that does not significantly alter tissue properties (see FIG. 30).

### Example 21

### Volumetric Properties Before and After Injection into Cervical Tissue

Cervical tissue was obtained from nonpregnant women undergoing hysterectomy for benign indications (IRB #8315). Samples (10 mm diameter×8 mm height) were treated with 2 mg/mL collagenase (˜0.4 U/mL) for 2 hrs at 37° C. Volumetric changes were calculating by determining the differences in the diameter and height after injecting 300 μL of silk-HA hydrogel. The hydrogel injection was visualized via hematoxylin and eosin (H&E) staining. In brief, the injected tissue was fixed in 10% phosphate buffered formlin, embedded in paraffin, and sectioned. H&E staining was performed using standard protocols by Tufts Medical Center histology lab.

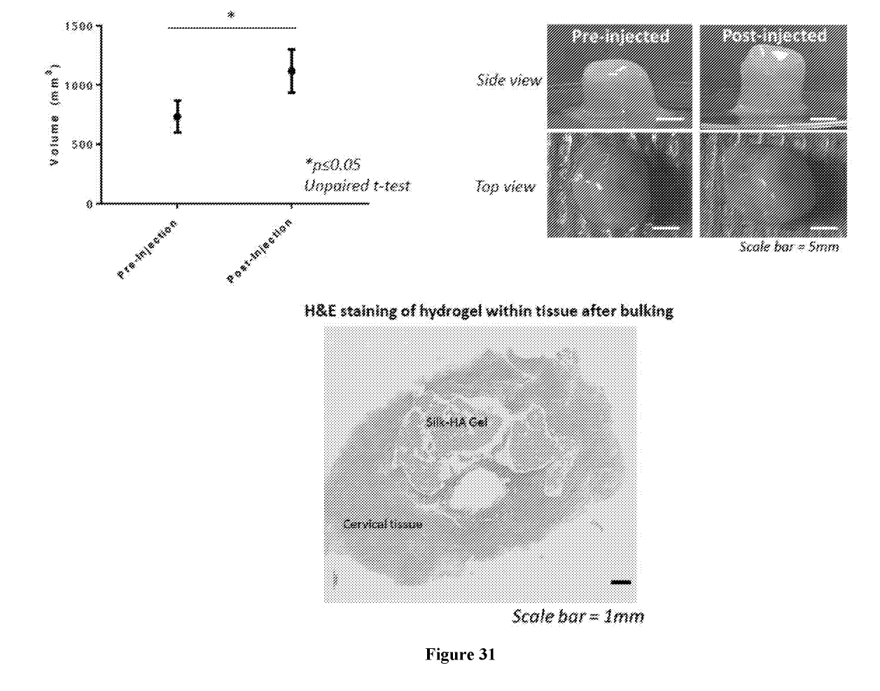

After injection there was an increase in volume, showing the potential of the hydrogels to bulk cervical tissue in the prevention of preterm birth. In addition, the hydrogel remained localized as seen through H&E staining (see FIG. 31).

### Example 22

### Viability of 2D Cervical Fibroblasts on Hydrogels

Cervical fibroblasts were isolated from hysterectomy specimens as previously described. Isolated cells were cultured in Dulbecco Modified Eagle Medium (DMEM) supplemented with 10% fetal bovine serum (FBS) and 1% antibiotic/antimycotic solution. Cells were plated on 300 μL of the hydrogel placed in a 24-well plate at a final density of 10,000 cells/cm². Cell viability was performed using LIVE/DEAD® Cell imaging kit as described above at days 1, 3, and 5.

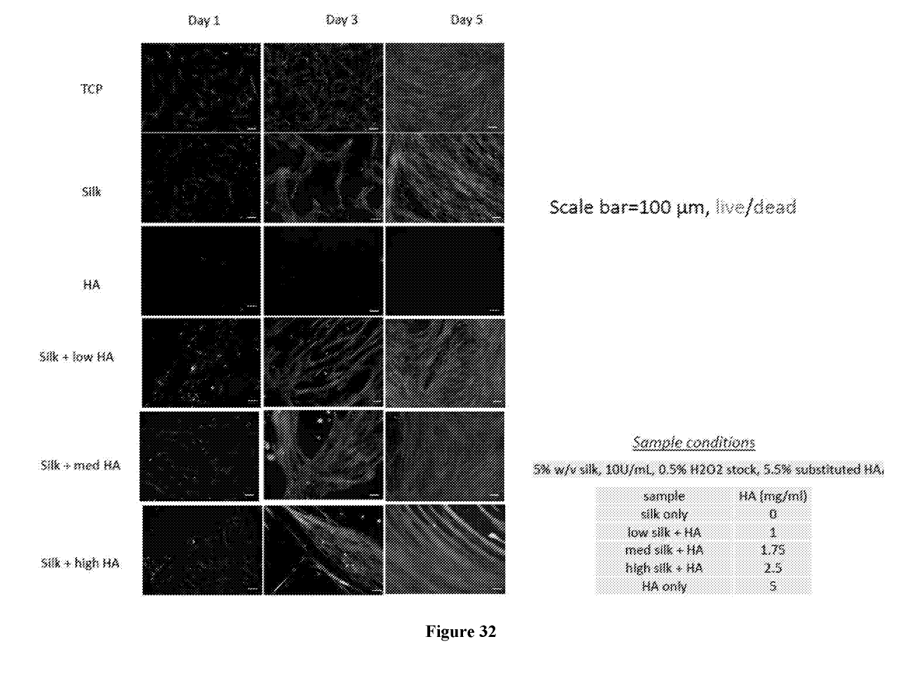

All composite (silk-HA) hydrogels supported cell growth with limited cell death, showing the cytocompatiblity of the hydrogels (see FIG. 32).

### Example 23

### Metabolic Activity and Proliferation of 2D Cervical Fibroblasts on Hydrogels

Cells were plated onto hydrogels as described above. Metabolic activity was assessed with AlamarBlue on days 1, 3, and 5 as per manufacturer's instructions. In brief, cells were incubated in a 1× AlamarBlue solution for 4 hours at 37 C. Fluorescence was read at excitation/emission of 560/590 nm with a microplate reader. Cell proliferation was quantified with Quanti-IT™ PicGreen dsDNA kit on days 1, 3, and 5 as per manufacturer's instructions.

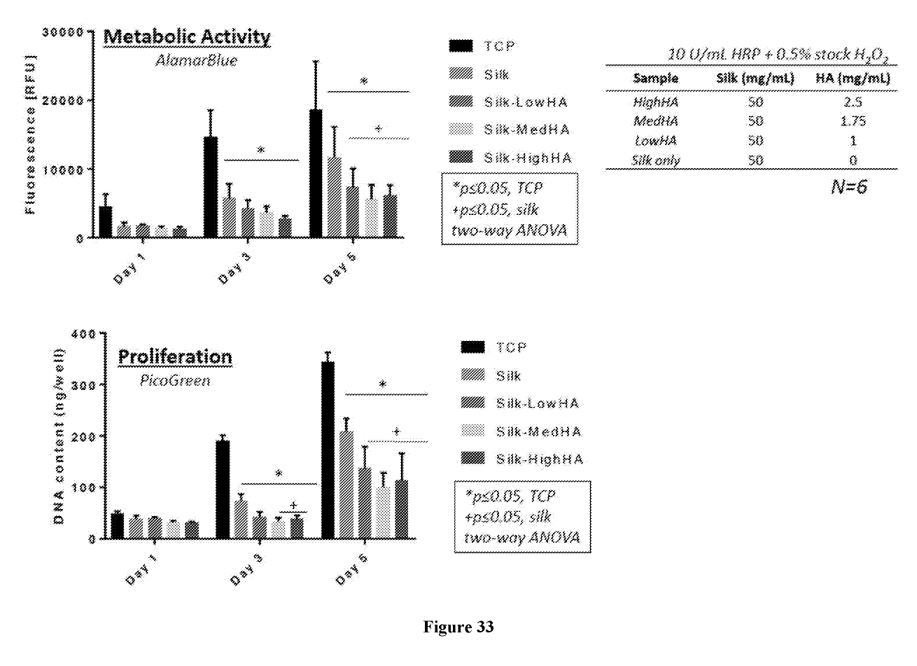

Cell on hydrogels exhibited an increase in metabolic activity and DNA content between days 1 and 5, showing the hydrogels are cytocompatible and support cell proliferation (see FIG. 33).

### Example 24

### Cytokine Production at Baseline and after LPS Induction

Cells were plated onto hydrogels as described above. To determine baseline cytokine production, 4 days after plating, media was collected and stored at −20° C. To determine cytokine production after induction using lipopolysaccharide from E. Coli (LPS), 4 days after plating, cultures were induced with 0.01 μg/mL of LPS. After 24 hours, media was collected and stored at −20° C. The media collected was assessed for IL-6 and IL-8 using Human IL-6 and IL-8 ELISA kits as per manufacturer's instructions.

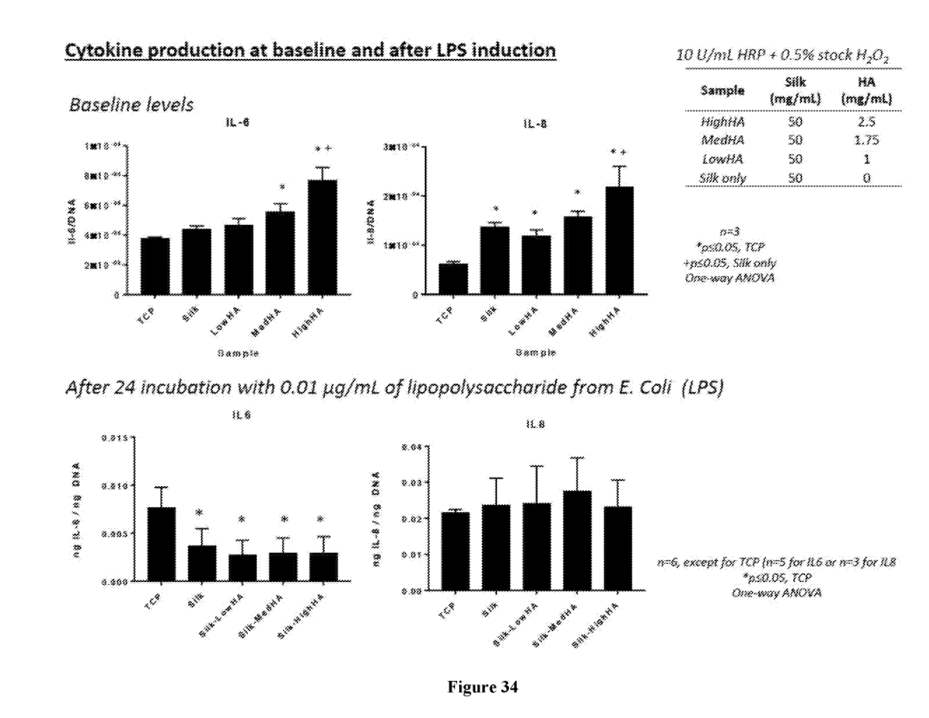

As is shown in FIG. 34, baseline cytokine levels were increased for the hydrogels with high HA concentrations. But upon inflammation (i.e. LPS induction), we see that the cells on hydrogels secrete similar amounts of IL-8 and decreased amounts of IL-6 as compared to tissue culture plastic controls. Further studies are necessary to gain an understanding of these results and conclusions are to be determined.

Table of samples tested for examples 16-24.

| | | | % tyramine | | |
|---|---|---|---|---|---|
| | Silk | TS-HA (mg/mL) | substitution | HRP | H2O2 |
| Experiment | (mg/mL) | (sample name) | on HA | (U/mL) | (% v/v) |
| I. SEM images | 20 | 0 (0%), 0.2 (1%), | 2.8 | 10 | 0.01 |
| | | 1.05 (5%), 2.22 | | | |
| | | (10%), 5.0 (20%, | | | |
| | | HA only) | | | |
| II. 3D hMSC | 20 | 0 (silk only), 2.22 | 2.8 | 10 | 0.01 |
| encapsulation without | | (silk-HA), 5.0 (HA | | | |
| DMEM | | only) | | | |
| III. CD44 | 20 | 0 (silk only), 2.22 | 2.8 | 10 | 0.01 |
| | | (silk-HA), 5.0 (HA | | | |
| IV. DAPI/Phalloidin | 20 | 0 (silk only), 2.22 | 2.8 | 10 | 0.01 |
| | | (silk-HA), 5.0 (HA | | | |
| V. Injection force | 50 | 0 (silk only), 1 (silk | 5 | 10 | 0.005 |
| | | low HA), 1.75 (silk | | | |
| | | med HA), 2.5 (silk | | | |
| | | high HA), 5 (HA | | | |
| | | only) | | | |
| VI. Cervical tissue | 50 | 1.75 | 5 | 10 | 0.005 |
| mechanics | | | | | |
| VII. Volumetric changes | 50 | 1.75 | 5 | 10 | 0.005 |
| VIII. Viability of cervical | 50 | 0 (silk only), 1 (silk | 5 | 10 | 0.005 |
| fibroblasts | | low HA), 1.75 (silk | | | |
| | | med HA), 2.5 (silk | | | |
| | | high HA), 5 (HA | | | |
| | | only) | | | |
| IX. Metabolic | 50 | 0 (silk only), 1 (silk | 5 | 10 | 0.005 |
| activity/proliferation of | | low HA), 1.75 (silk | | | |
| cervical fibroblasts | | med HA), 2.5 (silk | | | |
| | | high HA), 5 (HA | | | |
| | | only) | | | |
| X. Cytokine production | 50 | 0 (silk only), 1 (silk | 5 | 10 | 0.005 |
| | | low HA), 1.75 (silk | | | |
| | | med HA), 2.5 (silk | | | |
| | | high HA), 5 (HA | | | |
| | | only) | | | |

### Other Embodiments and Equivalents

While the present disclosures have been described in conjunction with various embodiments and examples, it is not intended that they be limited to such embodiments or examples. On the contrary, the disclosures encompass various alternatives, modifications, and equivalents, as will be appreciated by those of skill in the art. Accordingly, the descriptions, methods and diagrams of should not be read as limited to the described order of elements unless stated to that effect.

Although this disclosure has described and illustrated certain embodiments, it is to be understood that the disclosure is not restricted to those particular embodiments. Rather, the disclosure includes all embodiments that are functional and/or equivalents of the specific embodiments and features that have been described and illustrated.

We claim:

1. A composition comprising
silk fibroin; and
a phenol-containing polymer, wherein at least one tyrosine group of the silk fibroin is covalently crosslinked to at least one phenol group of the phenol-containing polymer.

2. The composition of claim 1, wherein the silk fibroin is selected from the group consisting of silkworm silk fibroin, spider silk fibroin, and recombinant silk fibroin.

3. The composition of claim 1 or claim 2, wherein the phenol-containing polymer is or comprises a peptide or protein.

4. The composition of any one of the above claims, wherein the phenol-containing polymer is or comprises a tyramine-containing and/or tyrosine-containing peptide or protein.

5. The composition of claim 4, wherein the tyramine-containing and/or tyrosine-containing polymer is or comprises hyaluronic acid and/or polyethylene glycol.

6. The composition of claim 4, wherein the tyramine-containing and/or tyrosine-containing polymer comprises a modified form (e.g., wherein one or more phenol or tyramine group(s) added) of one or more of: dopamine, L-DOPA, serotonin, adrenaline, noradrenaline, salicylic acid, alginate, dextran, collagen, gelatin, chitosan, carboxymethylcellulose, heparin, poly(vinyl alcohol), sugars (e.g., lactose, cellulose, mannose, galactose, glucose, maltose, etc) or dimers or trimers thereof.

7. The composition of any one of the above claims, wherein the composition is biocompatible.

8. The composition of any one of the above claims, wherein the composition is or comprises a hydrogel.

9. The composition of claim 8, wherein the composition further comprises at least one of an active agent and a plurality of particles.

10. The composition of any one of the above claims, wherein the composition has a compressive moduli of between 200 Pa and 1 MPa

11. The composition of any one of the above claims, wherein the composition has a compressive moduli of between 200 Pa and 500 kPa.

12. The composition of any one of the above claims, wherein the compositions has a mass fraction of at most 1.00 after soaking in an aqueous solution for 12 hours.

13. The composition of any one of claims, wherein the amount of phenol-containing polymer is between 2.5 mg/mL and 200 mg/mL.

14. The composition of any one of claims, wherein the amount of phenol-containing polymer is at most 8.5 mg/mL.

15. The composition of any one of the above claims, wherein the composition is formulated for injection.

16. The composition of claim 15, wherein the composition has an injection force of at most 40 N.

17. The composition of any one of the above claims, wherein the silk fibroin and phenol-containing polymer are crosslinked directly to one another (e.g., there is no spacer between the two).

18. The composition of any one of the above claims, wherein the crosslinking is or comprises multi-phenol crosslinks.

19. The composition of claim 18, wherein the multi-phenol crosslinks are selected from the group consisting of di-tyrosine crosslinks, di-tyramine crosslinks, and tyrosine-tyramine crosslinks.

20. The compositions of any one of the above claims, wherein the silk fibroin is modified to include at least one non-native tyrosine.

21. A method comprising
providing silk fibroin;
providing a phenol-containing polymer;
associating the silk fibroin with the phenol-containing polymer to form a mixed solution; and
crosslinking at least one tyrosine group in the silk fibroin and at least one phenol group of the phenol-containing polymer via at least one enzymatic reaction, wherein the crosslinking comprises covalent bonding between at least one tyrosine group of the silk fibroin and at least one phenol group of the phenol-containing polymer to form a crosslinked composition.

22. The method of claim 21, wherein the silk fibroin is selected from the group consisting of silkworm silk fibroin, spider silk fibroin, and recombinant silk fibroin.

23. The method of claim 21 or claim 22, wherein the phenol-containing polymer is or comprises a peptide or protein.

24. The method of any one of claims 21-23, wherein the phenol-containing polymer is or comprises a tyramine-containing and/or tyrosine-containing peptide or protein.

25. The method of claim 24, wherein the tyramine-containing and/or tyrosine-containing polymer is or comprises hyaluronic acid and/or polyethylene glycol.

26. The method of claim 24, wherein the tyramine-containing and/or tyrosine-containing polymer comprises a modified form (e.g., wherein one or more phenol or tyramine group(s) added) of one or more of: dopamine, L-DOPA, serotonin, adrenaline, noradrenaline, salicylic acid, alginate, dextran, collagen, gelatin, chitosan, carboxymethylcellulose, heparin, poly(vinyl alcohol), sugars (e.g., lactose, cellulose, mannose, galactose, glucose, maltose, etc) or dimers or trimers thereof.

27. The method of claim 24, wherein the tyramine-containing polymer is or comprises hyaluronic acid or polyethylene glycol.

28. The method of any of claims 21-27, wherein the crosslinked composition is biocompatible.

29. The method of any of claims 21-28, wherein the crosslinked composition is or comprises a hydrogel.

30. The method of claim 29, wherein the crosslinked composition comprises at least one of an active agent and a plurality of particles.

31. The method of claim 29 or 30, wherein the hydrogel exhibits a gelation time of between 10 seconds and 20 minutes after the crosslinking step.

32. The method of any one of claims 21-31, wherein the silk fibroin and phenol-containing polymer are each provided in a separate solution prior to the associating step.

33. The method of any one of claims 21-32, wherein the enzyme is or comprises a peroxidase.

34. The method of claim 33, wherein the peroxidase is or comprises a plant-based or mammal-based peroxidase.

35. The method of any one of claims 21-34, wherein the enzyme comprises at least one of hydrogen peroxide, tyrosinase, laccase, hemin, a microperoxidase, cytochrome c, porphyrins, fenton, soy bean peroxidase, myeloperoxidase, lactoperoxidase, eosinophil peroxidase, thyroid peroxidase, prostaglandin H synthase, and horseradish peroxidase.

36. The method of any one of claims 21-35, further comprising conjugating at least one additional tyrosine to the silk fibroin.

37. The method of any one of claims 21-36, wherein the crosslinking occurs directly between the silk fibroin and the phenol-containing polymer (e.g., there is no spacer between the two).

38. The method of any one of claims 21-37, wherein the crosslinking is or comprises multi-phenol crosslinks.

39. The method of claim 38, wherein the multi-phenol crosslinks are selected from the group consisting of di-tyrosine crosslinks, di-tyramine crosslinks, and tyrosine-tyramine crosslinks.
